(19) United States
(12) Patent Application Publication
(10) Pub. No.: US 2024/0174702 A1
(43) Pub. Date: May 30, 2024

(54) SOLID FORMS OF A COMPOUND FOR MODULATING COT

(71) Applicant: Gilead Sciences, Inc., Foster City, CA (US)

(72) Inventors: Ernest A. Carra, Foster City, CA (US); Lina Chan, Foster City, CA (US); Roland D. Saito, San Mateo, CA (US)

(21) Appl. No.: 18/314,989

(22) Filed: May 10, 2023

Related U.S. Application Data
(60) Provisional application No. 63/341,289, filed on May 12, 2022.

(51) Int. Cl. C07F 9/6558; A61K 45/06

(52) U.S. Cl. CPC C07F 9/65583; A61K 45/06; C07B 2200/13

(57) ABSTRACT

Disclosed herein are solid forms of a compound useful for modulating Cot. The disclosure also relates to pharmaceutical compositions containing one or more of the solid forms disclosed herein, as well as methods of using the solid forms in the treatment of conditions mediated by Cot. The disclosure also relates to methods for obtaining such solid forms.

### CROSS REFERENCE

This application claims priority to U.S. Provisional Application No. 63/341,289, filed May 12, 2022, which application is incorporated herein in its entirety for all purposes.

### FIELD

The present disclosure relates to solid forms of compounds useful for modulating Cot (cancer Osaka thyroid), and the pharmaceutical formulations and therapeutic uses thereof.

### BACKGROUND

Cot (cancer Osaka thyroid) protein is a serine/threonine kinase that is a member of the MAP kinase kinase kinase (MAP3K) family. It is also known as “Tp12” (tumor progression locus), “MAP3K8” (mitogen-activated protein kinase kinase kinase 8) or “EST” (Ewing sarcoma transformant). Cot was identified by its oncogenic transforming activity in cells and has been shown to regulate oncogenic and inflammatory pathways.

Cot is known to be upstream in the MEK-ERK pathway and is essential for LPS induced tumor necrosis factor-α (TNF-α) production. Cot has been shown to be involved in both production and signaling of TNFα. TNFα is a pro-inflammatory cytokine and plays an important role in inflammatory diseases, such as rheumatoid arthritis (RA), multiple sclerosis (MS), inflammatory bowel disease (IBD), diabetes, sepsis, psoriasis, mis-regulated TNFα expression and graft rejection.

Compounds that bind to the cancer Osaka thyroid (Cot) protein can act as modulators of Cot. Cot modulators are useful for the treatment and/or prophylaxis of diseases and conditions through binding of the Cot. One compound useful for modulating Cot is the compound of Formula I:

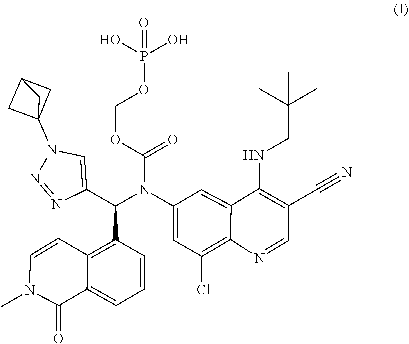 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

Although numerous compounds useful for modulating Cot are known, what is desired in the art are physically stable forms of the compound of Formula I, or pharmaceutically acceptable salt thereof, with desired properties such as good physical and chemical stability, good aqueous solubility and good bioavailability. For example, pharmaceutical compositions are desired that address challenges of stability, variable pharmacodynamics responses, drug-drug interactions, pH effects, food effects, and/or oral bioavailability.

Accordingly, there is a need for stable forms of a compound of Formula I with suitable chemical and physical stability for the formulation, therapeutic use, manufacturing, and storage of the compound.

A solid form may have properties such as bioavailability, stability, purity, and/or manufacturability at certain conditions that may be suitable for medical or pharmaceutical uses.

### SUMMARY

In some embodiments, the present disclosure is directed to solid forms of Formula I:

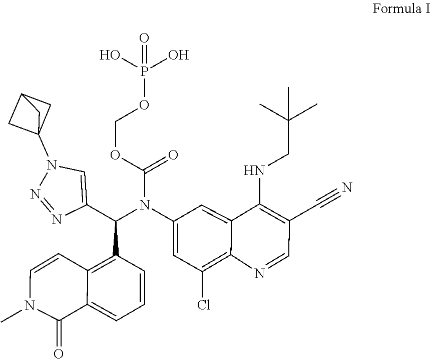 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

and pharmaceutically acceptable salts, solvates and hydrates thereof.

These forms are useful, for example, for treating human patients suffering from an inflammatory disease or condition, such as rheumatoid arthritis (RA), lupus, osteoarthritis (OA), or inflammatory bowel disease (IBD), such as ulcerative colitis (UC) or Crohn's disease (CD), Non-Alcoholic Steatohepatitis (NASH), primary sclerosing cholangitis (PSC), idiopathic pulmonary fibrosis (IPF), interstitial lung disease (ILD), diabetic kidney disease (DKD), or chronic kidney disease (CKD). The solid forms of the present disclosure can be useful for preparing a medicament for treating an inflammatory disease. The solid forms of the present disclosure can be used to modulate Cot.

In some embodiments, the present disclosure is directed to an amorphous Formula I.

In some embodiments, the present disclosure is directed to Formula I Form I.

In some embodiments, the present disclosure is directed to Formula I Form II.

In some embodiments, the present disclosure is directed to Formula I Form III.

In some embodiments, the present disclosure is directed to Formula I Form IV.

In some embodiments, the present disclosure is directed to Formula I Form V.

In some embodiments, the present disclosure is directed to Formula I Form VI.

In some embodiments, the present disclosure is directed to Formula I Form VII.

In some embodiments, the present disclosure is directed to Formula I Form VIII.

In some embodiments, the present disclosure is directed to Formula I Form IX.

In some embodiments, the present disclosure is directed to Formula I Form X.

In some embodiments, the present disclosure is directed to Formula I Form XI.

In some embodiments, the present disclosure is directed to Formula I Form XII.

In some embodiments, the present disclosure is directed to Formula I Form XIII.

In some embodiments, the present disclosure is directed to Formula I Form XIV.

In some embodiments, the present disclosure is directed to Formula I Form XV.

In some embodiments, the present disclosure is directed to Formula I 2-(4-Hydroxybenzoyl) benzoate.

In some embodiments, the present disclosure is directed to Formula I Vanillate.

In some embodiments, the present disclosure is directed to Formula I Hippurate.

In some embodiments, the present disclosure is directed to Formula I Maleate.

In some embodiments, the present disclosure is directed to Formula I Glyoxylate.

In some embodiments, the present disclosure is directed to Formula I L-Pyroglutamate.

In some embodiments, the present disclosure is directed to Formula I 2-Naphthalene Sulfonate.

In some embodiments, the present disclosure is directed to Formula I 1-Naphthalene Sulfonate.

In some embodiments, the present disclosure is directed to Formula I 1-Hydroxy-2-Naphthoate.

In some embodiments, the present disclosure is directed to Formula I S-Mandelate.

In some embodiments, the present disclosure is directed to Formula I Gentisate.

In some embodiments, the present disclosure is directed to Formula I Citrate.

In some embodiments, the present disclosure is directed to Formula I R-Mandelate.

In some embodiments, the present disclosure is directed to Formula I Benzoate.

In some embodiments, the present disclosure is directed to Formula I Methylparabenate.

In some embodiments, the present disclosure is directed to Formula I Caffeate.

In some embodiments, the present disclosure is directed to Formula I Glycolate.

In some embodiments, the present disclosure is directed to Formula I α-Ketobutyrate.

In some embodiments, the present disclosure is directed to Formula I Pyruvate.

In some embodiments, a pharmaceutical composition comprises a therapeutically effective amount of a solid form of Formula I.

### BRIEF DESCRIPTION OF THE FIGURES

FIG. 1 shows an XRPD pattern of Amorphous Formula I.

FIG. 2 shows a DSC thermogram of Amorphous Formula I.

FIG. 3 shows a TGA thermogram of Amorphous Formula I.

FIG. 4 shows a DVS isotherm of Amorphous Formula I.

FIG. 5 shows an XRPD pattern of Formula I Form I.

FIG. 6 shows a DSC thermogram of Formula I Form I.

FIG. 7 shows an XRPD pattern of Formula I Form II.

FIG. 8 shows a Whole Pattern Pawley Refinement of Formula I Form II (85% RH).

FIG. 9 shows a DVS isotherm of Formula I Form II.

FIG. 10 shows VH-XRD data depicting RH vs. Scans.

FIG. 11 shows an XRPD pattern of Formula I Form III (RH 36%; KF 2.3% water).

FIG. 12 shows VH-XRD data of Formula I Form III depicting XRPD taken at 40% RH.

FIG. 13 shows a Whole Pattern Pawley Refinement for Formula I Form III at 40% RH.

FIG. 14 shows a DVS isotherm of Formula I Form III.

FIG. 15 shows a Whole Pattern Pawley Refinement for Formula I Form IV at 0% RH.

FIG. 16 shows an XRPD pattern of Formula I Form IV at 5% RH.

FIG. 17 shows an XRPD pattern of Formula I Form V.

FIG. 18 shows a DSC thermogram of Formula I Form V.

FIG. 19 shows a XRPD pattern of Formula I Form VI.

FIG. 20 shows a magnified view of an XRPD pattern of Formula I Form VI.

FIG. 21 shows a DSC thermogram of Formula I Form VI.

FIG. 22 shows an XRPD pattern of Formula I Form VII.

FIG. 23 shows an XRPD pattern of Formula I Form VIII.

FIG. 24 shows an XRPD pattern of Formula I Form IX.

FIG. 25 shows an XRPD pattern of Formula I Form X.

FIG. 26 shows an XRPD pattern of Formula I Form XI.

FIG. 27 shows an XRPD pattern of Formula I Form XII.

FIG. 28 shows an XRPD pattern of Formula I Form XIII.

FIG. 29 shows an XRPD pattern of Formula I Form XIV.

FIG. 30 shows an XRPD pattern of Formula I Form XV.

FIG. 31 shows an XRPD pattern of Formula I 2-(4-Hydroxybenzoyl) Benzoate Form A.

FIG. 32 shows an XRPD pattern of Formula I 2-(4-Hydroxybenzoyl) Benzoate Form B.

FIG. 33 shows a DSC thermogram of Formula I 2-(4-Hydroxybenzoyl) Benzoate Form B.

FIG. 34 shows a TGA thermogram of Formula I 2-(4-Hydroxybenzoyl) Benzoate Form B.

FIG. 35 shows an XRPD pattern of Formula I Vanillate Form A.

FIG. 36 shows an XRPD pattern of Formula I Vanillate Form B.

FIG. 37 shows a DSC thermogram of Formula I Vanillate Form B.

FIG. 38 shows a TGA thermogram of Formula I Vanillate Form B.

FIG. 39 shows an XRPD pattern of Formula I Hippurate Form A.

FIG. 40 shows an XRPD pattern of Formula I Hippurate Form B.

FIG. 41 shows a DSC thermogram of Formula I Hippurate Form B.

FIG. 42 shows a TGA thermogram of Formula I Hippurate Form B.

FIG. 43 shows an XRPD pattern of Formula I Maleate Form A.

FIG. 44 shows an XRPD pattern of Formula I Maleate Form B.

FIG. 45 shows a DSC thermogram of Formula I Maleate Form B.

FIG. 46 shows a TGA thermogram of Formula I Maleate Form B.

FIG. 47 shows an XRPD pattern of Formula I Glyoxylate Form A.

FIG. 48 shows an XRPD pattern of Formula I Glyoxylate Form B.

FIG. 49 shows a DSC thermogram of Formula I Glyoxylate Form B.

FIG. 50 shows a TGA thermogram of Formula I Glyoxylate Form B.

FIG. 51 shows an XRPD pattern of Formula I L-Pyroglutamate (wet cake).

FIG. 52 shows a DSC thermogram of Formula I L-Pyroglutamate (air dried).

FIG. 53 shows an XRPD pattern of Formula I 2-Naphthalene Sulfonate (wet cake).

FIG. 54 shows an XRPD pattern of Formula I 2-Naphthalene Sulfonate (air dried).

FIG. 55 shows a DSC thermogram of Formula I 2-Naphthalene Sulfonate (air dried).

FIG. 56 shows a TGA thermogram of Formula I 2-Naphthalene Sulfonate (air dried).

FIG. 57 shows an XRPD pattern of Formula I 1-Naphthalene Sulfonate (wet cake).

FIG. 58 shows an XRPD pattern of Formula I 1-Naphthalene Sulfonate (air dried).

FIG. 59 shows a DSC thermogram of Formula I 1-Naphthalene Sulfonate (air dried).

FIG. 60 shows an XRPD pattern of Formula I 1-hydroxy-2-naphthoate (wet cake).

FIG. 61 shows an XRPD pattern of Formula I 1-hydroxy-2-naphthoate (air dried).

FIG. 62 shows a DSC thermogram of Formula I 1-hydroxy-2-naphthoate (air dried).

FIG. 63 shows a TGA thermogram of Formula I 1-hydroxy-2-naphthoate (air dried).

FIG. 64 shows an XRPD pattern of Formula I S-Mandelate Form A.

FIG. 65 shows an XRPD pattern of Formula I S-Mandelate Form B.

FIG. 66 shows a DSC thermogram of Formula I S-Mandelate Form B.

FIG. 67 shows a TGA thermogram of Formula I S-Mandelate Form B.

FIG. 68 shows an XRPD pattern of Formula I Gentisate (wet cake).

FIG. 69 shows an XRPD pattern of Formula I Gentisate (air dried).

FIG. 70 shows a DSC thermogram of Formula I Gentisate (air dried).

FIG. 71 shows a TGA thermogram of Formula I Gentisate (air dried).

FIG. 72 shows an XRPD pattern of Formula I Citrate (wet cake).

FIG. 73 shows an XRPD pattern of Formula I Citrate (air dried).

FIG. 74 shows a DSC thermogram of Formula I Citrate (air dried).

FIG. 75 shows a TGA thermogram of Formula I Citrate (air dried).

FIG. 76 shows an XRPD pattern of Formula I R-Mandelate Form A.

FIG. 77 shows an XRPD pattern of Formula I R-Mandelate Form B.

FIG. 78 shows a DSC thermogram of Formula I R-Mandelate Form B.

FIG. 79 shows a TGA thermogram of Formula I R-Mandelate Form B.

FIG. 80 shows an XRPD pattern of Formula I Benzoate Form A.

FIG. 81 shows an XRPD pattern of Formula I Benzoate Form B.

FIG. 82 shows a DSC thermogram of Formula I Benzoate Form B.

FIG. 83 shows a TGA thermogram of Formula I Benzoate Form B.

FIG. 84 shows an XRPD pattern of Formula I Methylparabenate Form A.

FIG. 85 shows an XRPD pattern of Formula I Methylparabenate Form B.

FIG. 86 shows a DSC thermogram of Formula I Methylparabenate Form B.

FIG. 87 shows a TGA thermogram of Formula I Methylparabenate Form B.

FIG. 88 shows an XRPD pattern of Formula I Caffeate (wet cake).

FIG. 89 shows an XRPD pattern of Formula I Caffeate (air dried).

FIG. 90 shows a DSC thermogram of Formula I Caffeate (air dried).

FIG. 91 shows a TGA thermogram of Formula I Caffeate (air dried).

FIG. 92 shows an XRPD pattern of Formula I Glycolate wet cake.

FIG. 93 shows an XRPD pattern of dried Formula I Glycolate.

FIG. 94 shows an XRPD pattern of Formula I α-Ketobutyrate wet cake.

FIG. 95 shows an XRPD pattern of dried Formula I α-Ketobutyrate.

FIG. 96 shows an XRPD pattern of Formula I Pyruvate wet cake.

FIG. 97 shows an XRPD pattern of dried Formula I Pyruvate.

### DETAILED DESCRIPTION

In the following description, certain specific details are set forth in order to provide a thorough understanding of various embodiments of the disclosure. However, one skilled in the art will understand that the disclosure may be practiced without these details. The description below of several embodiments is made with the understanding that the present disclosure is to be considered as an exemplification of the claimed subject matter and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### Definitions

Unless the context requires otherwise, throughout the present specification and claims, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Embodiments that reference throughout this specification to “a compound” include the crystalline, salt, co-crystal, hydrate, solvate, and/or amorphous forms of the formulas and/or compounds disclosed herein. Thus, the appearance or the phrase “a compound of Formula I” can include amorphous Formula I; Formula I Form I; Formula I Form II; Formula I Form III; Formula I Form IV; Formula I Form V; Formula I Form VI; Formula I Form VII; Formula I Form VIII; Formula I Form IX; Formula I Form X; Formula I Form XI; Formula I Form XII; Formula I Form XIII; Formula I Form XIV; Formula I Form XV; Formula I 2-(4-Hydroxybenzoyl) benzoate; Formula I Vanillate; Formula I Hippurate; Formula I Maleate; Formula I glyoxylate; Formula I L-Pyroglutamate; Formula I 2-Naphthalene Sulfonate; Formula I 1-Naphthalene Sulfonate; Formula I 1-Hydroxy-2-Naphthoate; Formula I S-Mandelate; Formula I Gentisate; Formula I Citrate; Formula I R-Mandelate; Formula I Benzoate; Formula I Methylparabenate; Formula I Caffeate; Formula I Glycolate; Formula I α-Ketobutyrate; and/or Formula I Pyruvate.

The disclosure disclosed herein is also meant to encompass all pharmaceutically acceptable compounds of Formula I being isotopically labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labeled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability. For example, in vivo half-life may increase, or dosage requirements may be reduced. Thus, heavier isotopes may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically labeled compounds of Formula I can generally be prepared by conventional techniques known to those skilled in the art or by processes analogous to those described in the Examples as set out below using an appropriate isotopically labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, and/or emulsifier, or a combination of one or more of the above which has been approved by the United States Food and Drug Administration as being acceptable for use in humans or domestic animals.

A “pharmaceutical composition” refers to a formulation of a compound of the disclosure (e.g., a compound of Formula I) and a medium generally accepted in the art for the delivery of the biologically active compound to mammals, e.g., humans. Such a medium includes all pharmaceutically acceptable excipients therefor.

“Effective amount” or “therapeutically effective amount” refers to an amount of a compound according to the disclosure, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the disclosure which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the disclosure, and the age, body weight, general health, sex and diet of the patient. Such a therapeutically effective amount can be determined routinely by one of ordinary skill in the art having regard to their own knowledge, the state of the art, and this disclosure.

“Prevention” or “preventing” or “prophylaxis” means any treatment of a disease or condition that causes the clinical symptoms of the disease or condition not to develop. Compounds may, in some embodiments, be administered to a subject (including a human) who is at risk or has a family history of the disease or condition.

“Treating” and “treatment” of a disease include the following:
(1) preventing or reducing the risk of developing the disease, i.e., causing the clinical symptoms of the disease not to develop in a subject that may be exposed to or predisposed to the disease but does not yet experience or display symptoms of the disease,
(2) inhibiting the disease, i.e., arresting or reducing the development of the disease or its clinical symptoms, and
(3) relieving the disease, i.e., causing regression of the disease or its clinical symptoms.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g., dogs and cats), farm animals (e.g., cattle, horses, sheep, goats, and pigs), and/or laboratory animals (e.g., mice, rats, hamsters, guinea pigs, pigs, rabbits, dogs, and monkeys). In some embodiments, the subject (or the patient) is a human. “Human (or patient) in need thereof” refers to a human who may have or is suspected of having diseases or conditions that would benefit from certain treatment; for example, being treated with the compounds disclosed herein according to the present application.

Reference to “about” a value or parameter herein includes (and describes) embodiments that are directed to that value or parameter per se. For example, description referring to “about X” includes description of “X.” Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

“Unit dosage forms” are physically discrete units suitable as unitary dosages for subjects (e.g., human subjects and other mammals), each unit containing a predetermined quantity of active material calculated to produce the desired therapeutic effect, in association with a suitable pharmaceutical excipient.

The term “substantially as shown in” when referring, for example, to an XRPD pattern, a DSC thermogram, a DVS isotherm, a TGA-MS thermogram, or a TGA thermogram includes a pattern, thermogram or spectrum that is not necessarily identical to those depicted herein, but that falls within the limits of experimental error or deviations when considered by one of ordinary skill in the art.

In some embodiments, the term “substantially pure” or “substantially free” with respect to a particular crystalline form of a compound means that the composition comprising the crystalline form contains less than 99%, less than 95%, less than 90%, less than 85%, less than 80%, less than 75%, less than 70%, less than 65%, less than 60%, less than 55%, less than 50%, less than 40%, less than 30%, less than 20%, less than 15%, less than 10%, less than 5%, or less than 1% by weight of other substances, including other crystalline forms and/or impurities. In certain embodiments, “substantially pure” or “substantially free of” refers to a substance free of other substances, including other crystalline forms and/or impurities. Impurities may, for example, include by-products or left-over reagents from chemical reactions, contaminants, degradation products, other crystalline forms, water, and solvents.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound. When the solvent is water, the “solvate” is a “hydrate.” A “solvate” may also be formed through interaction with ambient environment and starting material. Solvents are generally known to persons skilled in the art and can include, for example, methanol, ethanol, ethanol/water, acetone, tetrahydrofuran, dichloromethane, methyl t-butyl ether, 2-propanol, 1-propanol, and cyclopentyl methyl ether.

In certain embodiments, provided are optical isomers, racemates, or other mixtures thereof of the compounds described herein or a pharmaceutically acceptable salt or a mixture thereof. In some embodiments, isomers can be separated by methods well known in the art, e.g. by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposable mirror images of one another. “Diastereomers” are stereoisomers that have at least two asymmetric atoms, but which are not mirror-images of each other.

The compounds disclosed herein and their pharmaceutically acceptable salts may include an asymmetric center and may thus give rise to enantiomers, diastereomers, and other stereoisomeric forms that may be defined, in terms of absolute stereochemistry, as (R) or (S) or, as (D) or (L) for amino acids. The present invention is meant to include all such possible isomers, as well as their racemic and optically pure forms. Optically active (+) and ( ), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers.

Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

### Solid Forms of Formula I

Solid forms of Formula I, including crystalline forms and substantially pure forms, may provide the advantage of bioavailability and stability, suitable for use as an active ingredient in a pharmaceutical composition. Development of a suitable solid form for use in pharmaceutical compositions requires considerations of stability and bioavailability in varying environments. For example, in the case of a pharmaceutical drug product or an active ingredient, it may be desirable to exhibit stability at a pH below 5. Formula I Form II, III and IV, for example, exhibit advantageous physical properties such as good physical and chemical stability, e.g. at pH 5 and below, good aqueous solubility, good pharmacokinetic property and/or good bioavailability. Moreover, consideration should be given to conversions between solid forms under varying conditions. For instance, a humidity shift may result in conversion from one form to another. Care should be given to ensure that a desired form exhibits desirable properties under the conditions of interest. Under different humidity, conversions among Form II, III and IV could occur. Desirably, Formula I exhibits desirable stability under differing humidity conditions. Variations in the crystal structure of a pharmaceutical drug substance or active ingredient can, in some cases, affect dissolution rate, bioavailability, manufacturability (including for instance ease of handling, ability to consistently prepare doses of known strength), and stability (such as thermal stability, shelf life, and the like) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, solid forms of the compound of Formula I may provide advantages such as improving: the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different solid forms of the compound Formula I described herein which may exhibit one or more favorable characteristics described above. The processes for the preparation of the solid forms described herein and characterization of these solid forms are described in detail below.

In particular embodiments, solid forms, such as crystalline forms of Formula I are disclosed. In some embodiments, a solid form of amorphous Formula I is disclosed. In some embodiments, a solid form of Formula I Form I is disclosed. In some embodiments, a solid form of Formula I Form II is disclosed. In some embodiments, a solid form of Formula I Form III is disclosed. In some embodiments, a solid form of Formula I Form IV is disclosed. In some embodiments, a solid form of Formula I Form V is disclosed. In some embodiments, a solid form of Formula I Form VI is disclosed. In some embodiments, a solid form of Formula I Form VII is disclosed. In some embodiments, a solid form of Formula I Form VIII is disclosed. In some embodiments, a solid form of Formula I Form IX is disclosed. In some embodiments, a solid form of Formula I Form X is disclosed. In some embodiments, a solid form of Formula I Form XI is disclosed. In some embodiments, a solid form of Formula I Form XII is disclosed. In some embodiments, a solid form of Formula I Form XIII is disclosed I. In some embodiments, a solid form of Formula I Form XIV is disclosed. In some embodiments, a solid form of Formula I Form XV is disclosed. In some embodiments, a solid form of Formula I 2-(4-Hydroxybenzoyl) benzoate Form A is disclosed. In some embodiments, a solid form of Formula I 2-(4-Hydroxybenzoyl) benzoate Form B is disclosed. In some embodiments, a solid form of Formula I Vanillate Form A is disclosed. In some embodiments, a solid form of Formula I Vanillate Form B is disclosed. In some embodiments, a solid form of Formula I Hippurate Form A is disclosed. In some embodiments, a solid form of Formula I Hippurate Form B is disclosed. In some embodiments, a solid form of Formula I Maleate Form A is disclosed. In some embodiments, a solid form of Formula I Maleate Form B is disclosed. In some embodiments, a solid form of Formula I Glyoxylate Form A is disclosed. In some embodiments, a solid form of Formula I Glyoxylate Form B is disclosed. In some embodiments, a solid form of Formula I L-Pyroglutamate is disclosed. In some embodiments, a solid form of Formula I 2-Naphthalene sulfonate is disclosed. In some embodiments, a solid form of Formula I 1-Naphthalene sulfonate is disclosed. In some embodiments, a solid form of Formula I 1-Hydroxy-2-Naphthoate is disclosed. In some embodiments, a solid form of Formula I S-Mandelate Form A is disclosed. In some embodiments, a solid form of Formula I S-Mandelate Form B is disclosed. In some embodiments, a solid form of Formula I Gentisate is disclosed. In some embodiments, a solid form of Formula I Citrate is disclosed. In some embodiments, a solid form of Formula I R-Mandelate Form A is disclosed. In some embodiments, a solid form of Formula I R-Mandelate Form B is disclosed. In some embodiments, a solid form of Formula I Benzoate Form A is disclosed. In some embodiments, a solid form of Formula I Benzoate Form B is disclosed. In some embodiments, a solid form of Formula I Methylparabenate Form A is disclosed. In some embodiments, a solid form of Formula I Methylparabenate Form B is disclosed. In some embodiments, a solid form of Formula I Caffeate is disclosed. In some embodiments, a solid form of Formula I Glycolate is disclosed. In some embodiments, a solid form of Formula I α-Ketobutyrate is disclosed. In some embodiments, a solid form of Formula I Pyruvate is disclosed.

In some embodiments, crystalline salt and/or co-crystals including Formula I are provided. In some embodiments, crystalline salts and/or co-crystals including Formula I are derived from hydroxy benzoyl benzoic acid, Vanillic acid, gentisic acid, hippuric acid, maleic acid, glyoxylic acid, 2-naphthalene sulfonic acid, 1-naphthalene sulfonic acid, 1-hydroxy-2-naphthoic acid, S-Mandelic acid, citric acid, R-Mandelic acid, benzoic acid, methylparaben, caffeic acid, glycolic acid, α-ketobutyric acid, and pyruvic acid.

### Amorphous Formula I

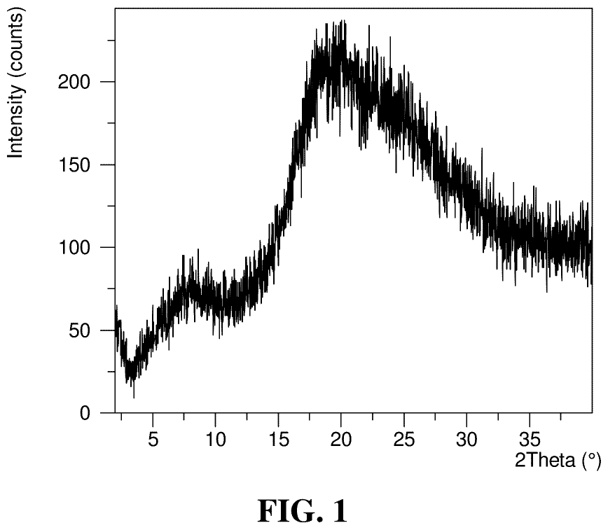

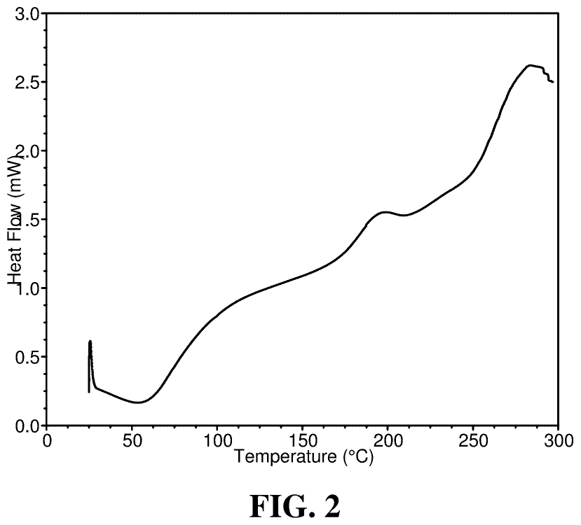

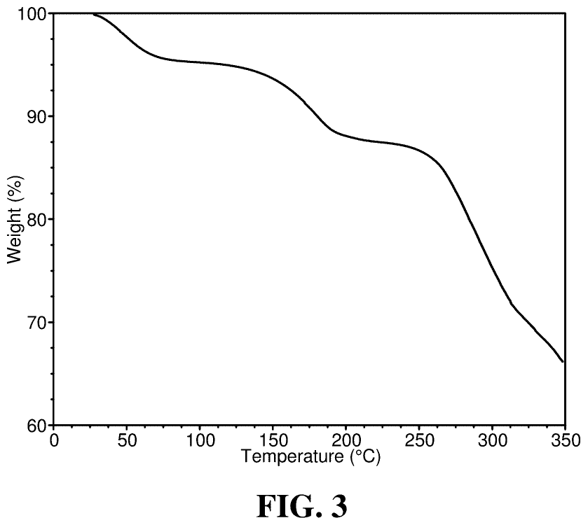

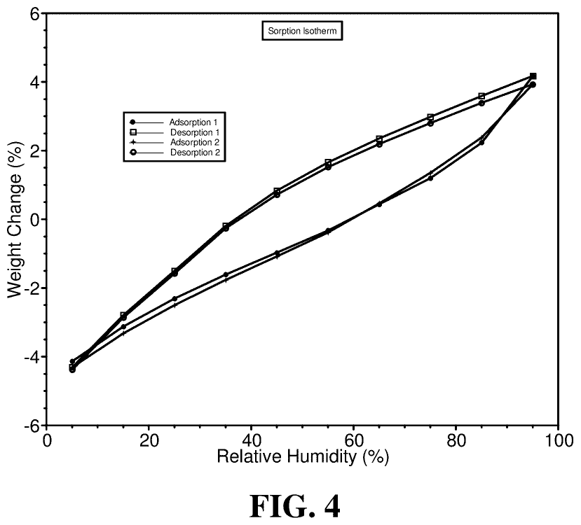

In some embodiments, provided herein is an amorphous solid compound of Formula I (Amorphous Formula I). In a further embodiment, Amorphous Formula I exhibits an X-ray powder diffraction (XRPD) pattern substantially as shown in FIG. 1. Amorphous Formula I may exhibit a differential scanning calorimetry (DSC) thermogram substantially as shown in FIG. 2. Amorphous Formula I may exhibit a thermogravimetric analysis (TGA) thermogram substantially as shown in FIG. 3. Amorphous Formula I may exhibit a dynamic vapor sorption (DVS) isotherm substantially as shown in FIG. 4

In some embodiments of amorphous Formula I, at least one, at least two, at least three, or all of the following (a)-(d) apply: (a) amorphous Formula I has an XRPD pattern substantially as shown in FIG. 1; (b) amorphous Formula I has a DSC thermogram substantially as shown in FIG. 2; (c) amorphous Formula I has a TGA thermogram substantially as shown in FIG. 3; (d) amorphous Formula I has a DVS isotherm substantially as shown in FIG. 4.

### Formula I Form I

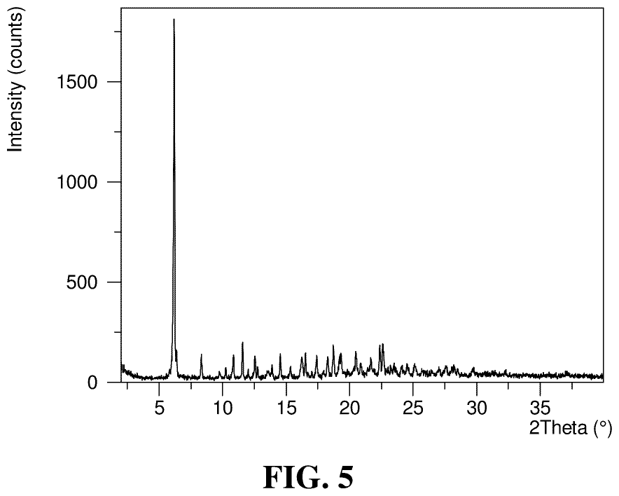

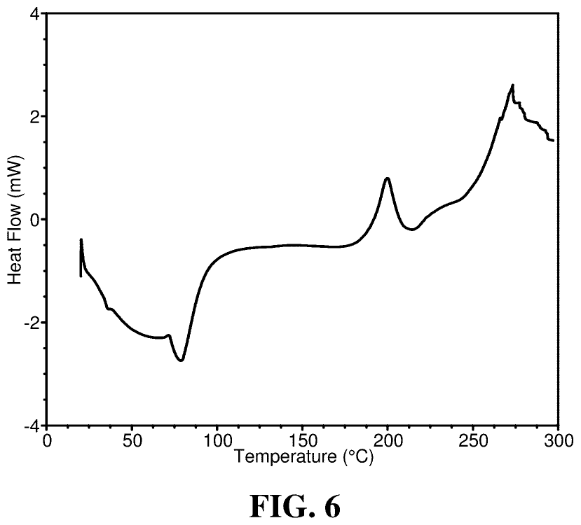

In some embodiments, provided herein is a solid form of Formula I Form I. In a further embodiment, Formula I Form I exhibits an X-ray powder diffraction (XRPD) pattern substantially as shown in FIG. 5. Formula I Form I may exhibit a differential scanning calorimetry (DSC) thermogram substantially as shown in FIG. 6.

In some embodiments, Formula I Form I has one or both of following properties:
(a) an XRPD pattern substantially as shown in FIG. 5
(b) a DSC thermogram substantially as shown in FIG. 6.

In some embodiments, Formula I Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 5.

In certain embodiments, Formula I Form I has an XRPD pattern comprising degree 2θ-reflections (±0.2 degrees 2θ) at 6.2, 8.3, and 11.6 degrees. In some embodiments, Formula I Form I has an XRPD pattern comprising degree 2θ-reflections (±0.2 degrees 2θ) at 6.2, 8.3, and 11.6 degrees and one, two, or three of the degree 2θ-reflections (±0.2 degrees 2θ) at 10.9, 14.5, and 22.4 degrees. In some embodiments, Formula I Form I has an XRPD pattern comprising degree 2θ-reflections (±0.2 degrees 2θ) at 6.2, 8.3, and 11.6 degrees and one, two, or three of the degree 2θ-reflections (±0.2 degrees 2θ) at 17.4, 18.7, and 22.6 degrees. In some embodiments, Formula I Form I has an XRPD pattern comprising degree 2θ-reflections (±0.2 degrees 2θ) at 6.2, 8.3, 11.6, 10.9, 14.5, 22.4, 17.4, 18.7, and 22.6 degrees.

In some embodiments, Formula I Form I has a differential scanning calorimetry thermogram having an endotherm with onset at about 25° C. In some embodiments, Formula I Form I has a differential scanning calorimetry thermogram having an exotherm with onset at about 175° C.

### Formula I Form II

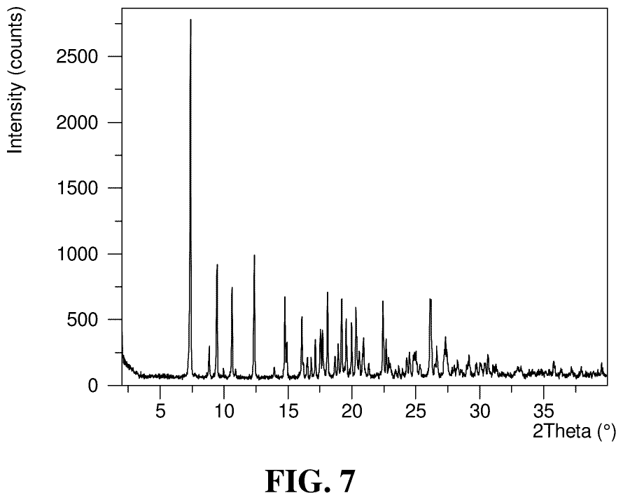

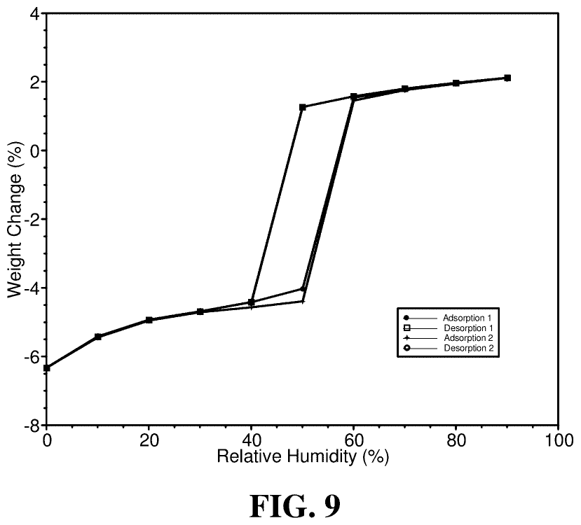

In some embodiments, provided herein is a solid form of Formula I Form II, wherein the solid form exhibits an X-ray powder diffraction (XRPD) pattern substantially as shown in FIG. 7. Formula I Form II may exhibit a dynamic vapor sorption (DVS) isotherm substantially as shown in FIG. 9.

In some embodiments of Formula I Form II, one or both of the following (a)-(b) apply: (a) Formula I Form II has an XRPD pattern substantially as shown in FIG. 7; (b) Formula I Form II has a DVS isotherm substantially as shown in FIG. 9.

In some embodiments, Formula I Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 7.

In certain embodiments, Formula I Form II has an XRPD pattern comprising degree 2θ-reflections (±0.2 degrees 2θ) at 7.4, 9.4, and 10.6 degrees. In some embodiments, Formula I Form II has an XRPD pattern comprising degree 2θ-reflections (±0.2 degrees 2θ) at 7.4, 9.4, and 10.6 degrees and one, two, or three of the degree 2θ-reflections (±0.2 degrees 2θ) at 8.8, 12.3, and 26.1 degrees. In some embodiments, Formula I Form II has an XRPD pattern comprising degree 2θ-reflections (±0.2 degrees 2θ) at 7.4, 9.4, and 10.6 degrees and one, two, or three of the degree 2θ-reflections (±0.2 degrees 2θ) at 14.7, 18.1, and 22.4 degrees. In some embodiments, Formula I Form I has an XRPD pattern comprising degree 2θ-reflections (±0.2 degrees 2θ) at 7.4, 9.4, 10.6, 8.8, 12.3, 26.1, 14.7, 18.1, and 22.4 degrees.

### Formula I Form III

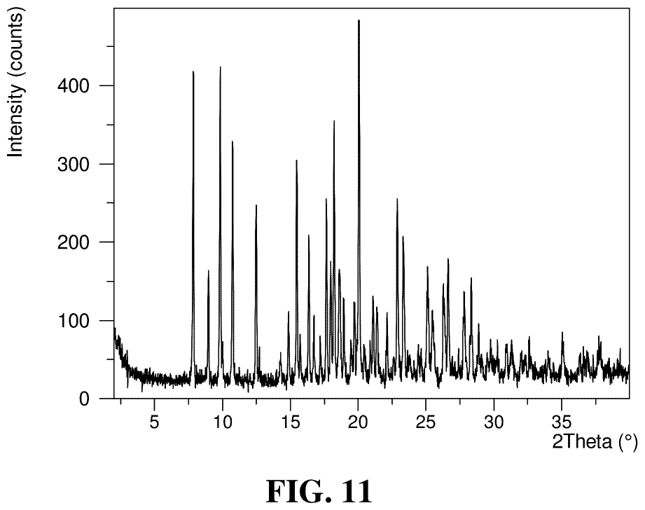

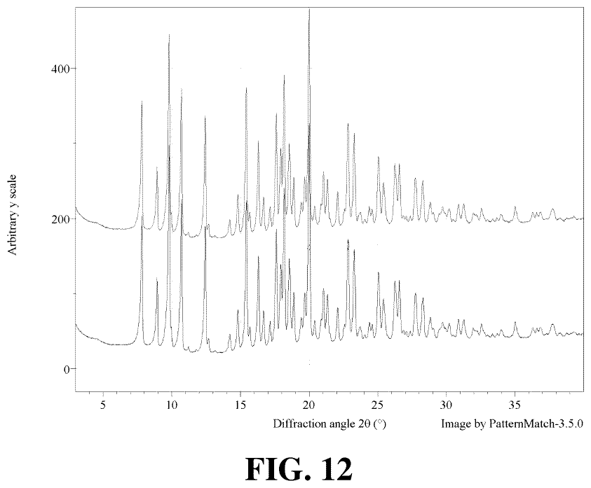

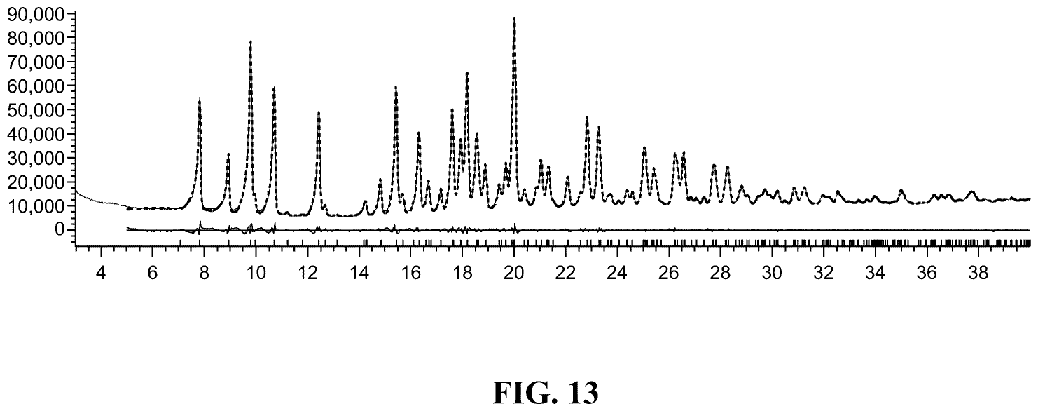

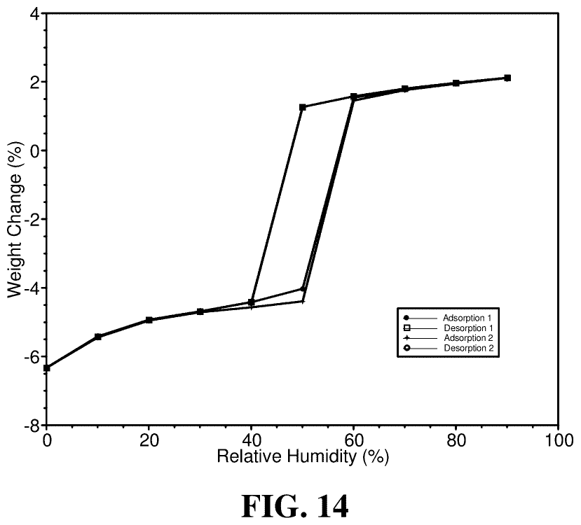

In some embodiments, provided herein is a Formula I Form III, wherein the solid form exhibits an X-ray powder diffraction (XRPD) pattern substantially as shown in FIG. 11. Formula I Form III may exhibit a VH-XRD pattern substantially as shown in FIG. 12. Formula I Form III may exhibit a Pawley Refinement pattern substantially as shown in FIG. 13. Formula I Form III may exhibit a dynamic vapor sorption (DVS) isotherm substantially as shown in FIG. 14.

In some embodiments, Formula I Form III has at least one, at least two, at least three, or at least four of the following properties:
(a) an XRPD pattern substantially as shown in FIG. 11
(b) a VH-XRD pattern substantially as shown in FIG. 12
(c) a Pawley Refinement pattern XRPD substantially as shown in FIG. 13
(d) a DVS isotherm substantially as shown in FIG. 14.

In some embodiments, Formula I Form III has an XRPD pattern displaying at least two, at least three, at least four, at least five, at least six, at least seven, at least eight, or at least nine of the degree 2θ-reflections with the greatest intensity as the XRPD pattern substantially as shown in FIG. 11.

In certain embodiments, Formula I Form III has an XRPD pattern comprising degree 2θ-reflections (±0.2 degrees 2θ) at 7.8, 9.8, and 10.7 degrees. In some embodiments, Formula I Form III has an XRPD pattern comprising degree 2θ-reflections (±0.2 degrees 2θ) at 7.8, 9.8, and 10.7 degrees and one, two, or three of the degree 2θ-reflections (±0.2 degrees 2θ) at 8.9, 12.5, and 20.1 degrees. In some embodiments, Formula I Form III has an XRPD pattern comprising degree 2θ-reflections (±0.2 degrees 2θ) at 7.8, 9.8, and 10.7 degrees and one, two, or three of the degree 2θ-reflections (±0.2 degrees 2θ) at 15.5, 18.2, and 22.9 degrees. In some embodiments, Formula I Form III has an XRPD pattern comprising degree 2θ-reflections (±0.2 degrees 2θ) at 7.8, 9.8, 10.7, 8.9, 12.5, 20.1, 15.5, 18.2, and 22.9 degrees.

### Formula I Form IV

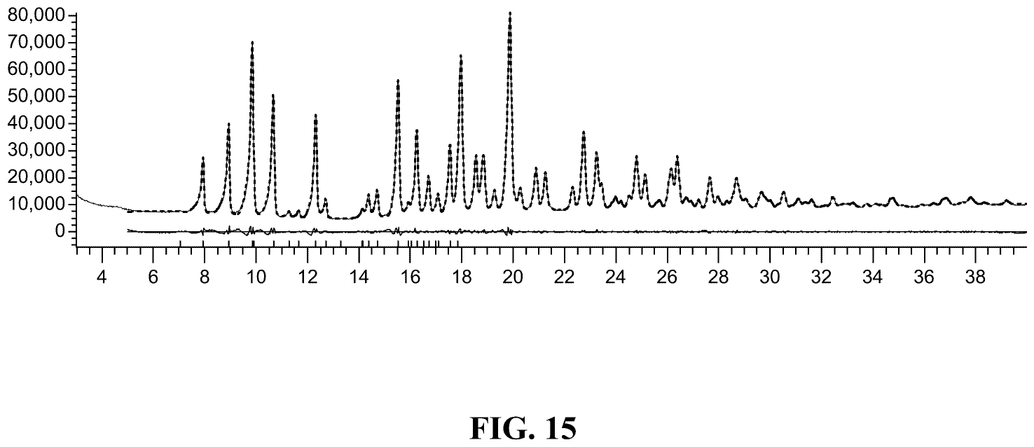

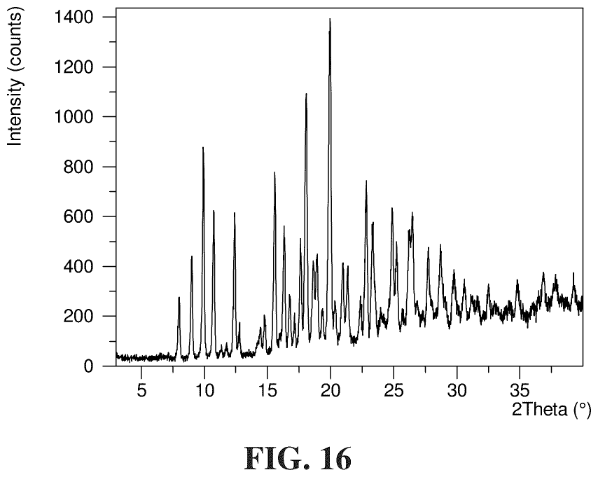

In some embodiments, provided herein is a Formula I Form IV, wherein the solid form exhibits an X-ray powder diffraction (XRPD) pattern substantially as shown in FIG. 16. Formula I Form IV may exhibit a Pawley Refinement XRPD pattern substantially as shown in FIG. 15.

In some embodiments, Formula I Form IV has an XRPD pattern displaying at least two, at least three, at least four, at least five, at least six, at least seven, at least eight, or at least nine of the degree 2θ-reflections with the greatest intensity as the XRPD pattern substantially as shown in FIG. 16.

In certain embodiments, Formula I Form IV has an XRPD pattern comprising degree 2θ-reflections (±0.2 degrees 2θ) at 8.0, 18.1, and 20.0 degrees. In some embodiments, Formula I Form IV has an XRPD pattern comprising degree 2θ-reflections (±0.2 degrees 2θ) at 8.0, 18.1, and 20.0 degrees and one, two, or three of the degree 2θ-reflections (±0.2 degrees 2θ) at 9.0, 9.9, and 10.8 degrees. In some embodiments, Formula I Form IV has an XRPD pattern comprising degree 2θ-reflections (±0.2 degrees 2θ) at 8.0, 18.1, and 20.0 degrees and one, two, or three of the degree 2θ-reflections (±0.2 degrees 2θ) at 15.6, 22.8, and 24.9 degrees. In some embodiments, Formula I Form IV has an XRPD pattern comprising degree 2θ-reflections (±0.2 degrees 2θ) at 8.0, 18.1, 20.0, 9.0, 9.9, 10.8, 15.6, 22.8, and 24.9 degrees.

### Formula I Form V

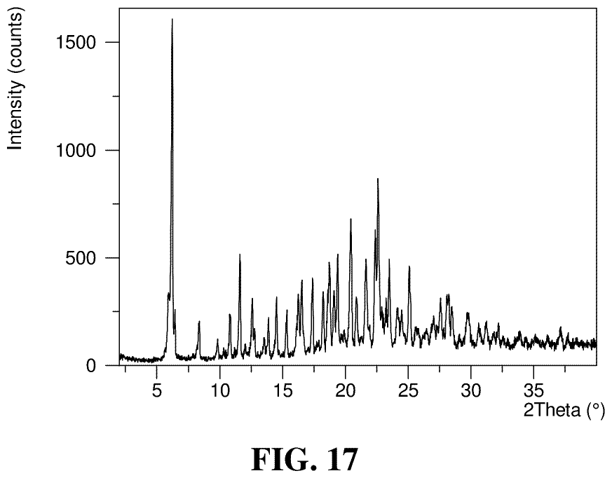

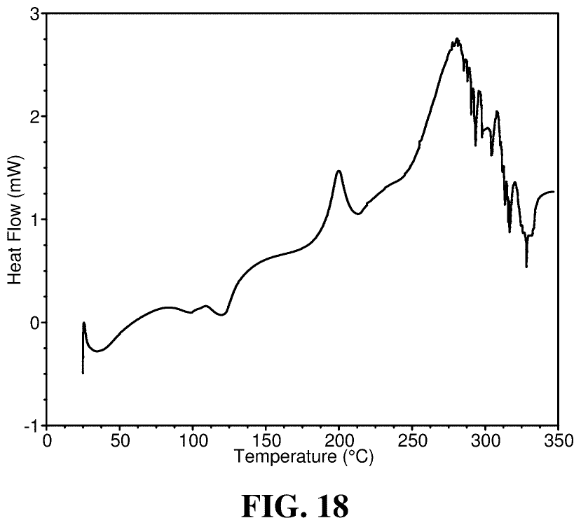

In some embodiments, provided herein is a Formula I Form V, wherein the solid form exhibits an X-ray powder diffraction (XRPD) pattern substantially as shown in FIG. 17. Formula I Form V may exhibit a differential scanning calorimetry (DSC) thermogram substantially as shown in FIG. 18.

In some embodiments of Formula I Form V, one or both of the following (a)-(b) apply: (a) Formula I Form V has an XRPD pattern substantially as shown in FIG. 17; (b) Formula I Form V has a DSC thermogram substantially as shown in FIG. 18.

In some embodiments, Formula I Form V has an XRPD pattern displaying at least two, at least three, at least four, at least five, at least six, at least seven, at least eight, or at least nine of the degree 2θ-reflections with the greatest intensity as the XRPD pattern substantially as shown in FIG. 17.

In certain embodiments, Formula I Form V has an XRPD pattern comprising degree 2θ-reflections (±0.2 degrees 2θ) at 6.2, 22.6, and 20.4 degrees. In some embodiments, Formula I Form V has an XRPD pattern comprising degree 2θ-reflections (±0.2 degrees 2θ) at 6.2, 22.6, and 20.4 degrees and one, two, or three of the degree 2θ-reflections (±0.2 degrees 2θ) at 16.3, 16.5, and 17.4 degrees. In some embodiments, Formula I Form V has an XRPD pattern comprising degree 2θ-reflections (±0.2 degrees 2θ) at 6.2, 22.6, and 20.4 degrees and one, two, or three of the degree 2θ-reflections (±0.2 degrees 2θ) at 22.4, 23.5, and 25.1 degrees. In some embodiments, Formula I Form V has an XRPD pattern comprising degree 2θ-reflections (±0.2 degrees 2θ) at 6.2, 22.6, 20.4, 16.3, 16.5, 17.4, 22.4, 23.5, and 25.1 degrees.

### Formula I Form VI

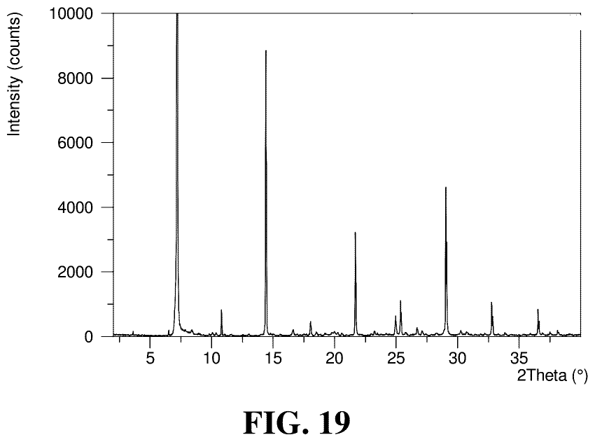

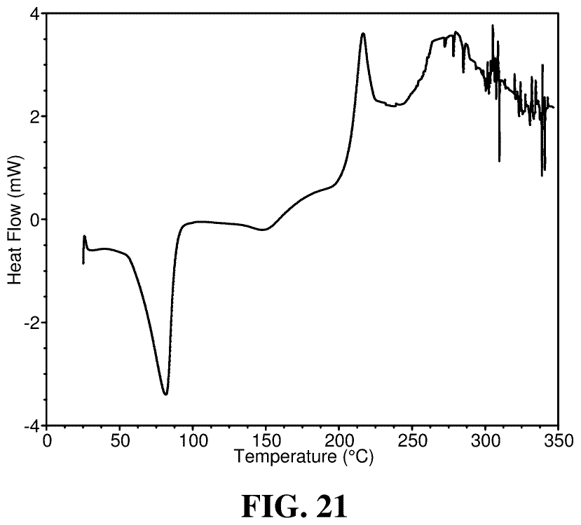

In some embodiments, provided herein is a Formula I Form VI, wherein the solid form exhibits an X-ray powder diffraction (XRPD) pattern substantially as shown in FIG. 19. Formula I Form VI may exhibit a differential scanning calorimetry (DSC) thermogram substantially as shown in FIG. 21.

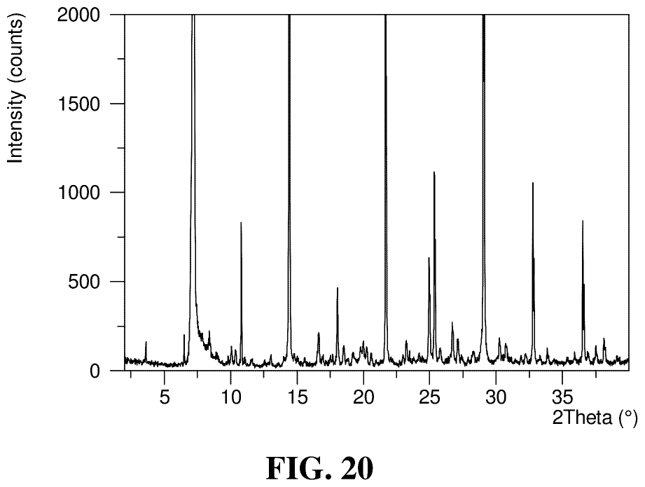

In some embodiments of Formula I Form VI, one or both of the following (a)-(b) apply: (a) Formula I Form VI has an XRPD pattern substantially as shown in FIG. 19 and FIG. 20 (a magnified image); (b) Formula I Form VI has a DSC thermogram substantially as shown in FIG. 21.

In some embodiments, Formula I Form VI has an XRPD pattern displaying at least two, at least three, at least four, at least five, at least six, at least seven, at least eight, or at least nine of the degree 2θ-reflections with the greatest intensity as the XRPD pattern substantially as shown in FIG. 19.

In certain embodiments, Formula I Form VI has an XRPD pattern comprising degree 2θ-reflections (±0.2 degrees 2θ) at 7.2, 14.4, and 21.7 degrees. In some embodiments, Formula I Form VI has an XRPD pattern comprising degree 2θ-reflections (±0.2 degrees 2θ) at 7.2, 14.4, and 21.7 degrees and one, two, or three of the degree 2θ-reflections (±0.2 degrees 2θ) at 29.1, 25.3, and 25.0 degrees. In some embodiments, Formula I Form VI has an XRPD pattern comprising degree 2θ-reflections (±0.2 degrees 2θ) at 7.2, 14.4, and 21.7 degrees and one, two, or three of the degree 2θ-reflections (±0.2 degrees 2θ) at 16.6, 26.7, and 30.2 degrees. In some embodiments, Formula I Form VI has an XRPD pattern comprising degree 2θ-reflections (±0.2 degrees 2θ) at 7.2, 14.4, 21.7, 29.1, 25.3, 25.0, 16.6, 26.7, and 30.2 degrees.

### Formula I Form VII

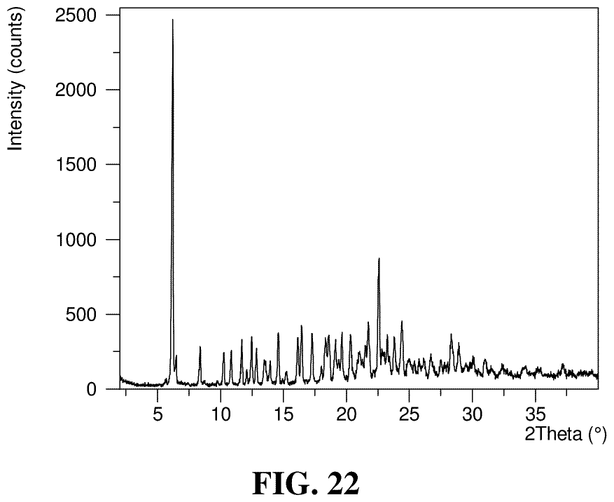

In some embodiments, provided herein is a Formula I Form VII, wherein the solid form exhibits an X-ray powder diffraction (XRPD) pattern substantially as shown in FIG. 22.

In some embodiments, Formula I Form VII has an XRPD pattern displaying at least two, at least three, at least four, at least five, at least six, at least seven, at least eight, or at least nine of the degree 2θ-reflections with the greatest intensity as the XRPD pattern substantially as shown in FIG. 22.

In certain embodiments, Formula I Form VII has an XRPD pattern comprising degree 2θ-reflections (±0.2 degrees 2θ) at 6.2, 8.4, and 22.6 degrees. In some embodiments, Formula I Form VII has an XRPD pattern comprising degree 2θ-reflections (±0.2 degrees 2θ) at 6.2, 8.4, and 22.6 degrees and one, two, or three of the degree 2θ-reflections (±0.2 degrees 2θ) at 10.3, 10.9, and 11.7 degrees. In some embodiments, Formula I Form VII has an XRPD pattern comprising degree 2θ-reflections (±0.2 degrees 2θ) at 6.2, 8.4, and 22.6 degrees and one, two, or three of the degree 2θ-reflections (±0.2 degrees 2θ) at 16.1, 16.4, and 17.3 degrees. In some embodiments, Formula I Form VII has an XRPD pattern comprising degree 2θ-reflections (±0.2 degrees 2θ) at 6.2, 8.4, 22.6, 10.3, 10.9, 11.7, 16.1, 16.4, and 17.3 degrees.

### Formula I Form VIII

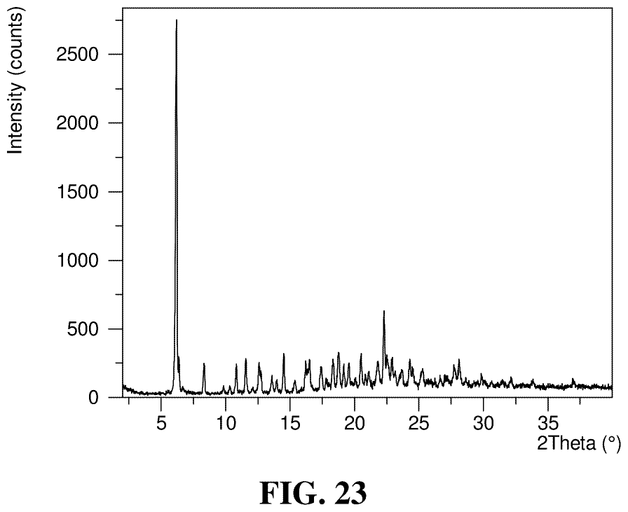

In some embodiments, provided herein is a Formula I Form VIII, wherein the solid form exhibits an X-ray powder diffraction (XRPD) pattern substantially as shown in FIG. 23.

In some embodiments, Formula I Form VIII has an XRPD pattern displaying at least two, at least three, at least four, at least five, at least six, at least seven, at least eight, or at least nine of the degree 2θ-reflections with the greatest intensity as the XRPD pattern substantially as shown in FIG. 23.

In certain embodiments, Formula I Form VIII has an XRPD pattern comprising degree 2θ-reflections (±0.2 degrees 2θ) at 6.2, 8.3, and 22.3 degrees. In some embodiments, Formula I Form VIII has an XRPD pattern comprising degree 2θ-reflections (±0.2 degrees 2θ) at 6.2, 8.3, and 22.3 degrees and one, two, or three of the degree 2θ-reflections (±0.2 degrees 2θ) at 11.6, 10.8, and 14.5 degrees. In some embodiments, Formula I Form VIII has an XRPD pattern comprising degree 2θ-reflections (±0.2 degrees 2θ) at 6.2, 8.3, and 22.3 degrees and one, two, or three of the degree 2θ-reflections (±0.2 degrees 2θ) at 16.5, 18.7, and 20.5 degrees. In some embodiments, Formula I Form VIII has an XRPD pattern comprising degree 2θ-reflections (±0.2 degrees 2θ) at 6.2, 8.3, 22.3, 11.6, 10.8, 14.5, 16.5, 18.7, and 20.5 degrees.

### Formula I Form IX

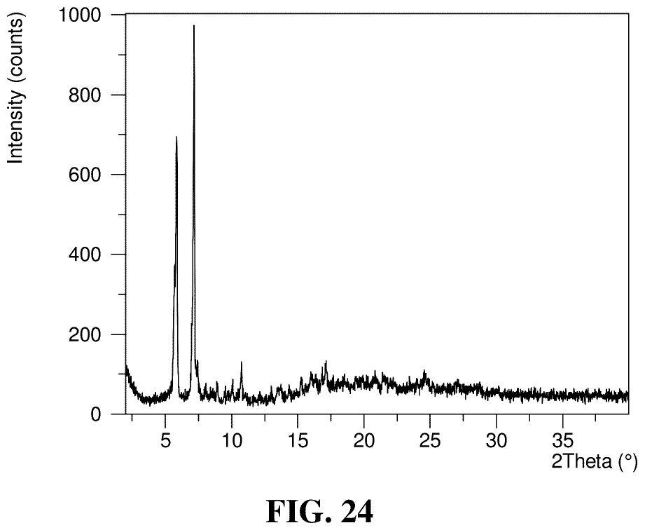

In some embodiments, provided herein is a Formula I Form IX, wherein the solid form exhibits an X-ray powder diffraction (XRPD) pattern substantially as shown in FIG. 24.

In some embodiments, Formula I Form IX has an XRPD pattern displaying at least two, at least three, at least four, at least five, at least six, at least seven, at least eight, or at least nine of the degree 2θ-reflections with the greatest intensity as the XRPD pattern substantially as shown in FIG. 24.

In certain embodiments, Formula I Form IX has an XRPD pattern comprising degree 2θ-reflections (±0.2 degrees 2θ) at 7.2, 5.8, and 5.7 degrees. In some embodiments, Formula I Form IX has an XRPD pattern comprising degree 2θ-reflections (±0.2 degrees 2θ) at 7.2, 5.8, and 5.7 degrees and one, two, or three of the degree 2θ-reflections (±0.2 degrees 2θ) at 10.7, 15.3, and 17.1 degrees. In some embodiments, Formula I Form IX has an XRPD pattern comprising degree 2θ-reflections (±0.2 degrees 2θ) at 7.2, 5.8, and 5.7 degrees and one, two, or three of the degree 2θ-reflections (±0.2 degrees 2θ) at 7.4, 10.0, and 8.9 degrees. In some embodiments, Formula I Form IX has an XRPD pattern comprising degree 2θ-reflections (±0.2 degrees 2θ) at 7.2, 5.8, 5.7, 10.7, 15.3, 17.1, 7.4, 10.0, and 8.9 degrees.

### Formula I Form X

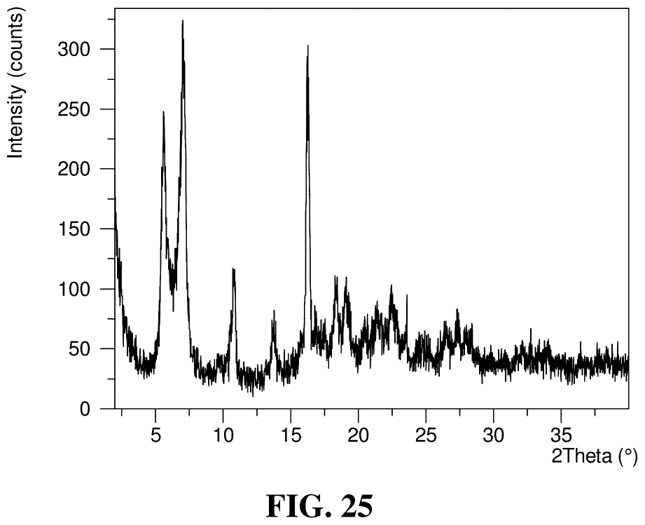

In some embodiments, provided herein is a Formula I Form X, wherein the solid form exhibits an X-ray powder diffraction (XRPD) pattern substantially as shown in FIG. 25.

In some embodiments, Formula I Form X has an XRPD pattern displaying at least two, at least three, at least four, at least five, at least six, at least seven, at least eight, or at least nine of the degree 2θ-reflections with the greatest intensity as the XRPD pattern substantially as shown in FIG. 25.

In certain embodiments, Formula I Form X has an XRPD pattern comprising degree 2θ-reflections (±0.2 degrees 2θ) at 16.3, 7.2, and 5.7 degrees. In some embodiments, Formula I Form X has an XRPD pattern comprising degree 2θ-reflections (±0.2 degrees 2θ) at 16.3, 7.2, and 5.7 degrees and one, two, or three of the degree 2θ-reflections (±0.2 degrees 2θ) at 10.8, 13.7, and 18.3 degrees. In some embodiments, Formula I Form X has an XRPD pattern comprising degree 2θ-reflections (±0.2 degrees 2θ) at 16.3, 7.2, and 5.7 degrees and one, two, or three of the degree 2θ-reflections (±0.2 degrees 2θ) at 19.1, 22.4, and 26.5 degrees. In some embodiments, Formula I Form X has an XRPD pattern comprising degree 2θ-reflections (±0.2 degrees 2θ) at 16.3, 7.2, 5.7, 10.8, 13.7, 18.3, 19.1, 22.4, and 26.5 degrees.

### Formula I Form XI

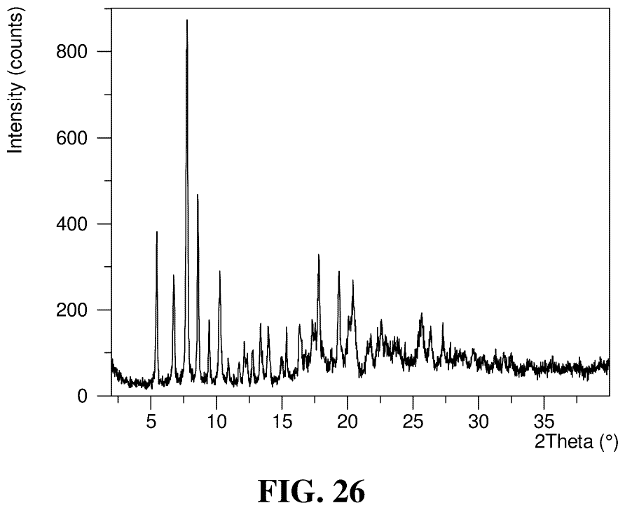

In some embodiments, provided herein is a Formula I Form XI, wherein the solid form exhibits an X-ray powder diffraction (XRPD) pattern substantially as shown in FIG. 26.

In some embodiments, Formula I Form XI has an XRPD pattern displaying at least two, at least three, at least four, at least five, at least six, at least seven, at least eight, or at least nine of the degree 2θ-reflections with the greatest intensity as the XRPD pattern substantially as shown in FIG. 26.

In certain embodiments, Formula I Form XI has an XRPD pattern comprising degree 2θ-reflections (±0.2 degrees 2θ) at 7.8, 5.5, and 6.8 degrees. In some embodiments, Formula I Form XI has an XRPD pattern comprising degree 2θ-reflections (±0.2 degrees 2θ) at 7.8, 5.5, and 6.8 degrees and one, two, or three of the degree 2θ-reflections (±0.2 degrees 2θ) at 8.6, 9.5, and 10.3 degrees. In some embodiments, Formula I Form XI has an XRPD pattern comprising degree 2θ-reflections (±0.2 degrees 2θ) at 7.8, 5.5, and 6.8 degrees and one, two, or three of the degree 2θ-reflections (±0.2 degrees 2θ) at 15.4, 17.8, and 19.4 degrees. In some embodiments, Formula I Form XI has an XRPD pattern comprising degree 2θ-reflections (±0.2 degrees 2θ) at 7.8, 5.5, 6.8, 8.6, 9.5, 10.3, 15.4, 17.8, and 19.4 degrees.

### Formula I Form XII

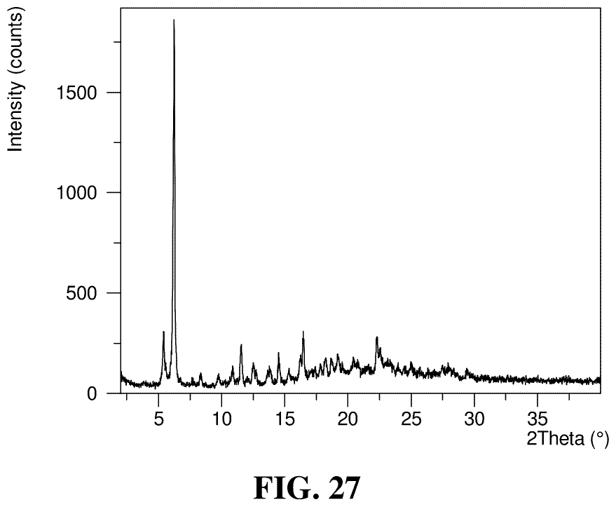

In some embodiments, provided herein is a Formula I Form XII, wherein the solid form exhibits an X-ray powder diffraction (XRPD) pattern substantially as shown in FIG. 27.

In some embodiments, Formula I Form XII has an XRPD pattern displaying at least two, at least three, at least four, at least five, at least six, at least seven, at least eight, or at least nine of the degree 2θ-reflections with the greatest intensity as the XRPD pattern substantially as shown in FIG. 27.

In certain embodiments, Formula I Form XII has an XRPD pattern comprising degree 2θ-reflections (±0.2 degrees 2θ) at 6.2, 5.4, and 11.5 degrees. In some embodiments, Formula I Form XII has an XRPD pattern comprising degree 2θ-reflections (±0.2 degrees 2θ) at 6.2, 5.4, and 11.5 degrees and one, two, or three of the degree 2θ-reflections (±0.2 degrees 2θ) at 14.5, 16.4, and 22.3 degrees. In some embodiments, Formula I Form XII has an XRPD pattern comprising degree 2θ-reflections (±0.2 degrees 2θ) at 6.2, 5.4, and 11.5 degrees and one, two, or three of the degree 2θ-reflections (±0.2 degrees 2θ) at 12.5, 9.7, and 19.2 degrees. In some embodiments, Formula I Form XII has an XRPD pattern comprising degree 2θ-reflections (±0.2 degrees 2θ) at 6.2, 5.4, 11.5, 14.5, 16.4, 22.3, 12.5, 9.7, and 19.2 degrees.

### Formula I Form XIII

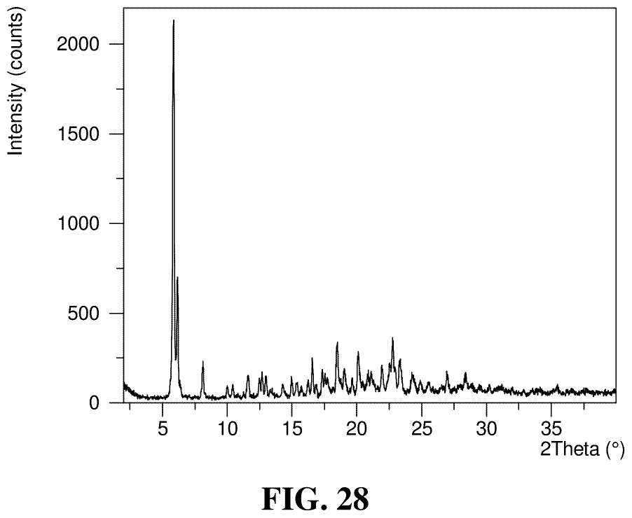

In some embodiments, provided herein is a Formula I Form XIII, wherein the solid form exhibits an X-ray powder diffraction (XRPD) pattern substantially as shown in FIG. 28.

In some embodiments, Formula I Form XIII has an XRPD pattern displaying at least two, at least three, at least four, at least five, at least six, at least seven, at least eight, or at least nine of the degree 2θ-reflections with the greatest intensity as the XRPD pattern substantially as shown in FIG. 28.

In certain embodiments, Formula I Form XIII has an XRPD pattern comprising degree 2θ-reflections (±0.2 degrees 2θ) at 5.8, 6.2, and 8.1 degrees. In some embodiments, Formula I Form XIII has an XRPD pattern comprising degree 2θ-reflections (±0.2 degrees 2θ) at 5.8, 6.2, and 8.1 degrees and one, two, or three of the degree 2θ-reflections (±0.2 degrees 2θ) at 11.6, 16.6, and 20.0 degrees. In some embodiments, Formula I Form XIII has an XRPD pattern comprising degree 2θ-reflections (±0.2 degrees 2θ) at 5.8, 6.2, and 8.1 degrees and one, two, or three of the degree 2θ-reflections (±0.2 degrees 2θ) at 13.0, 22.0, and 22.8 degrees. In some embodiments, Formula I Form XIII has an XRPD pattern comprising degree 2θ-reflections (±0.2 degrees 2θ) at 5.8, 6.2, 8.1, 11.6, 16.6, 20.0, 13.0, 22.0, and 22.8 degrees.

### Formula I Form XIV

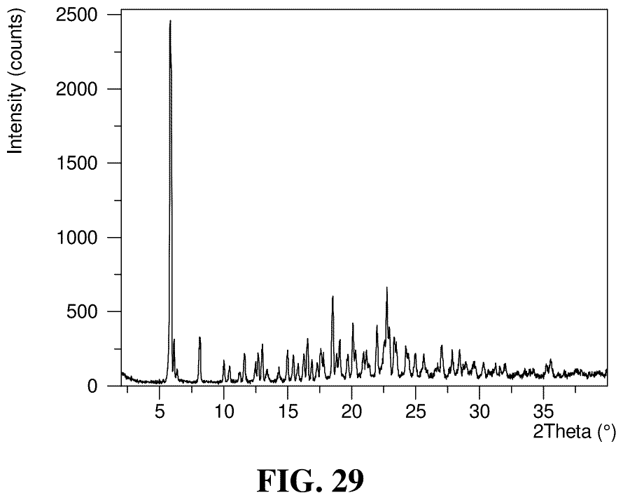

In some embodiments, provided herein is a Formula I Form XIV, wherein the solid form exhibits an X-ray powder diffraction (XRPD) pattern substantially as shown in FIG. 29.

In some embodiments, Formula I Form XIV has an XRPD pattern displaying at least two, at least three, at least four, at least five, at least six, at least seven, at least eight, or at least nine of the degree 2θ-reflections with the greatest intensity as the XRPD pattern substantially as shown in FIG. 29.

In certain embodiments, Formula I Form XIV has an XRPD pattern comprising degree 2θ-reflections (±0.2 degrees 2θ) at 5.8, 8.2, and 18.5 degrees. In some embodiments, Formula I Form XIV has an XRPD pattern comprising degree 2θ-reflections (±0.2 degrees 2θ) at 5.8, 8.2, and 18.5 degrees and one, two, or three of the degree 2θ-reflections (±0.2 degrees 2θ) at 11.7, 16.6, and 22.0 degrees. In some embodiments, Formula I Form XIV has an XRPD pattern comprising degree 2θ-reflections (±0.2 degrees 2θ) at 5.8, 8.2, and 18.5 degrees and one, two, or three of the degree 2θ-reflections (±0.2 degrees 2θ) at 22.8, 10.0, and 10.5 degrees. In some embodiments, Formula I Form XIV has an XRPD pattern comprising degree 2θ-reflections (±0.2 degrees 2θ) at 5.8, 8.2, 18.5, 11.7, 16.6, 22.0, 22.8, 10.0, and 10.5 degrees.

### Formula I Form XV

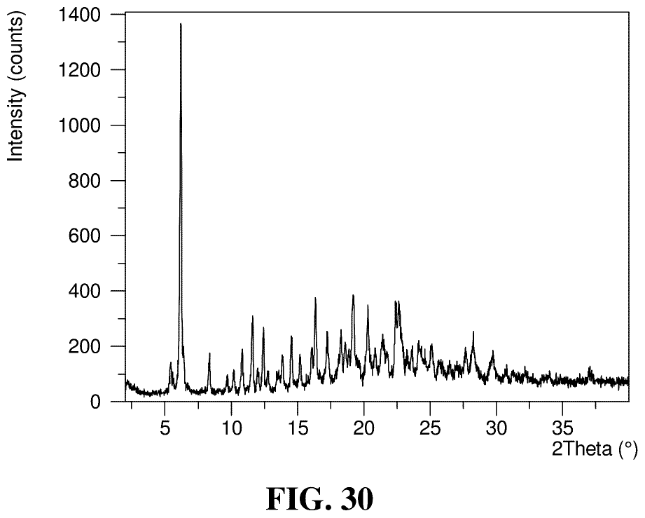

In some embodiments, provided herein is a Formula I Form XV, wherein the solid form exhibits an X-ray powder diffraction (XRPD) pattern substantially as shown in FIG. 30.

In some embodiments, Formula I Form XV has an XRPD pattern displaying at least two, at least three, at least four, at least five, at least six, at least seven, at least eight, or at least nine of the degree 2θ-reflections with the greatest intensity as the XRPD pattern substantially as shown in FIG. 30.

In certain embodiments, Formula I Form XV has an XRPD pattern comprising degree 2θ-reflections (±0.2 degrees 2θ) at 6.2, 5.4, and 8.3 degrees. In some embodiments, Formula I Form XV has an XRPD pattern comprising degree 2θ-reflections (±0.2 degrees 2θ) at 6.2, 5.4, and 8.3 degrees and one, two, or three of the degree 2θ-reflections (±0.2 degrees 2θ) at 11.6, 16.4, and 19.3 degrees. In some embodiments, Formula I Form XV has an XRPD pattern comprising degree 2θ-reflections (±0.2 degrees 2θ) at 6.2, 5.4, and 8.3 degrees and one, two, or three of the degree 2θ-reflections (±0.2 degrees 2θ) at 12.4, 20.3, and 22.4 degrees. In some embodiments, Formula I Form XV has an XRPD pattern comprising degree 2θ-reflections (±0.2 degrees 2θ) at 6.2, 5.4, 8.3, 11.6, 16.4, 19.3, 12.4, 20.3, and 22.4 degrees.

Formula I 2-(4-Hydroxybenzoyl) benzoate

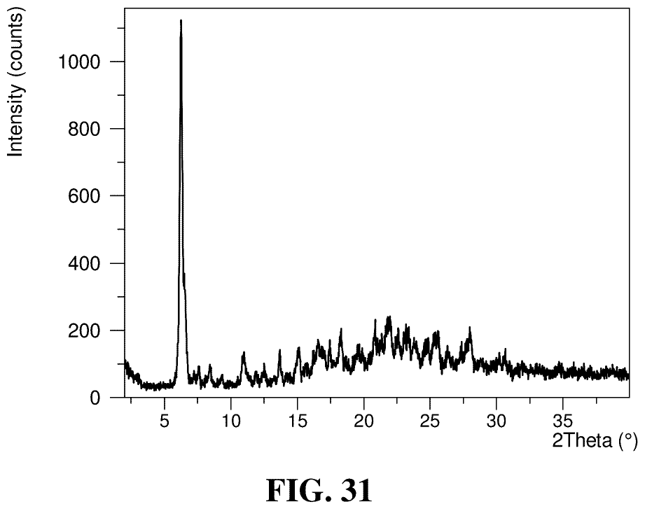

In some embodiments, provided herein is a Formula I 2-(4-Hydroxybenzoyl) benzoate Form A, wherein the solid form exhibits an X-ray powder diffraction (XRPD) pattern substantially as shown in FIG. 31.

In some embodiments, Formula I 2-(4-Hydroxybenzoyl) benzoate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 31.

In certain embodiments, Formula I 2-(4-Hydroxybenzoyl) benzoate Form A has an XRPD pattern comprising degree 2θ-reflections (±0.2 degrees 2θ) at 6.2, 11.0, and 13.7 degrees. In some embodiments, Formula I 2-(4-Hydroxybenzoyl) benzoate Form A has an XRPD pattern comprising degree 2θ-reflections (±0.2 degrees 2θ) at 6.2, 11.0, and 13.7 degrees and one, two, or three of the degree 2θ-reflections (±0.2 degrees 2θ) at 15.2, 17.4, and 18.3 degrees. In some embodiments, Formula I 2-(4-Hydroxybenzoyl) benzoate Form A has an XRPD pattern comprising degree 2θ-reflections (±0.2 degrees 2θ) at 6.2, 11.0, and 13.7 degrees and one, two, or three of the degree 2θ-reflections (±0.2 degrees 2θ) at 20.8, 7.6, and 8.5 degrees. In some embodiments, Formula I 2-(4-Hydroxybenzoyl) benzoate Form A has an XRPD pattern comprising degree 2θ-reflections (±0.2 degrees 2θ) at 6.2, 11.0, 13.7, 15.2, 17.4, 18.3, 20.8, 7.6, and 8.5 degrees.

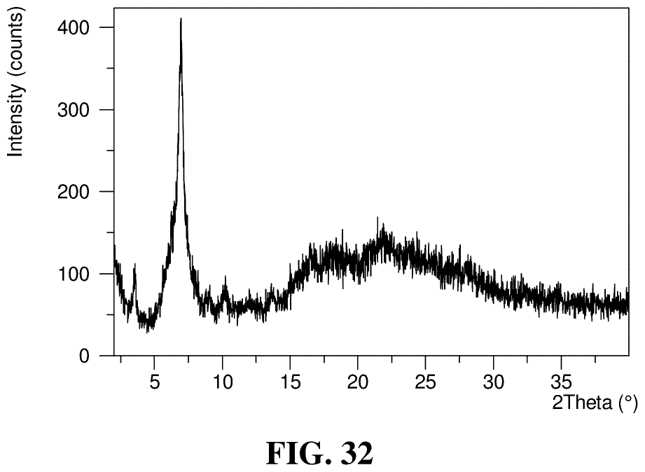

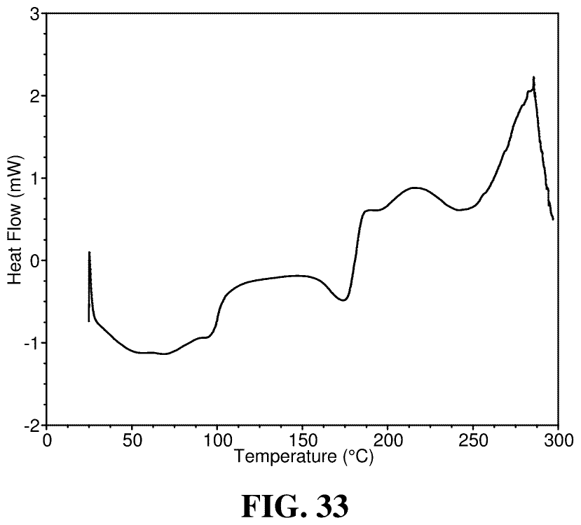

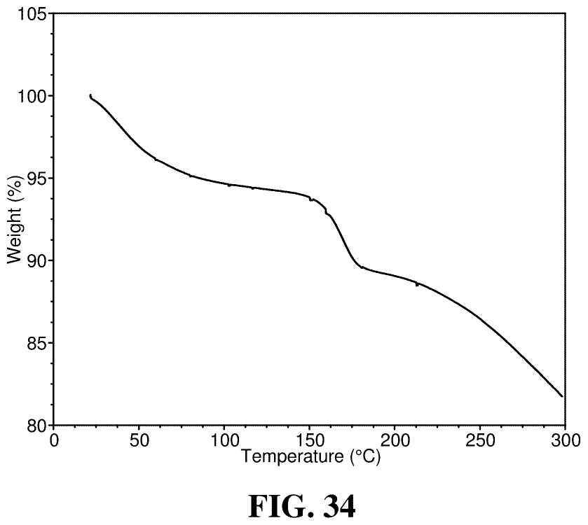

In some embodiments, provided herein is a Formula I 2-(4-Hydroxybenzoyl) benzoate Form B, wherein the solid form exhibits an X-ray powder diffraction (XRPD) pattern substantially as shown in FIG. 32. Formula I 2-(4-Hydroxybenzoyl) benzoate Form B may exhibit a differential scanning calorimetry (DSC) thermogram substantially as shown in FIG. 33. Formula I 2-(4-Hydroxybenzoyl) benzoate Form B may exhibit a thermogravimetric analysis (TGA) thermogram substantially as shown in FIG. 34.

In some embodiments, Formula I 2-(4-Hydroxybenzoyl) benzoate Form B has at least one, at least two, or at least three of the following properties:
(a) an XRPD pattern substantially as shown in FIG. 32
(b) a DSC thermogram substantially as shown in FIG. 33
(c) a TGA thermogram substantially as shown in FIG. 34.

### Formula I Vanillate

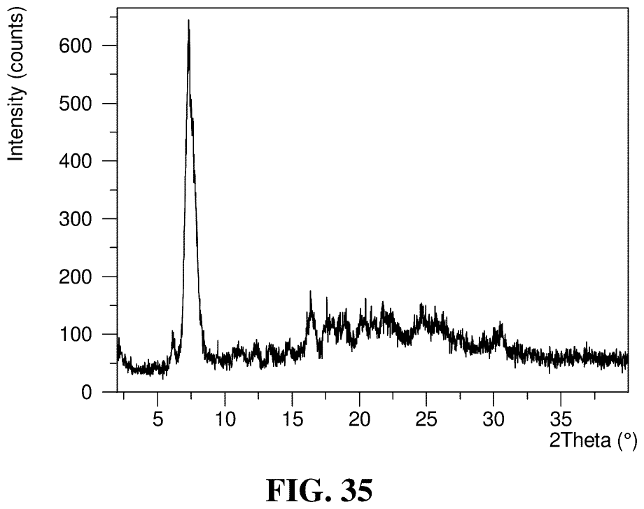

In some embodiments, provided herein is a Formula I Vanillate Form A, wherein the solid form exhibits an X-ray powder diffraction (XRPD) pattern substantially as shown in FIG. 35.

In some embodiments, Formula I Vanillate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 35.

In certain embodiments, Formula I Vanillate Form A has an XRPD pattern comprising degree 2θ-reflections (±0.2 degrees 2θ) at 6.2, 7.3, and 16.4 degrees. In some embodiments, Formula I Vanillate Form A has an XRPD pattern comprising degree 2θ-reflections (±0.2 degrees 2θ) at 6.2, 7.3, and 16.4 degrees and one, two, or three of the degree 2θ-reflections (±0.2 degrees 2θ) at 17.5, 24.7, and 30.6 degrees. In some embodiments, Formula I Vanillate Form A has an XRPD pattern comprising degree 2θ-reflections (±0.2 degrees 2θ) at 6.2, 7.3, and 16.4 degrees and one, two, or three of the degree 2θ-reflections (±0.2 degrees 2θ) at 12.4, 13.4, and 20.2 degrees. In some embodiments, Formula I Vanillate Form A has an XRPD pattern comprising degree 2θ-reflections (±0.2 degrees 2θ) at 6.2, 7.3, 16.4, 17.5, 24.7, 30.6, 12.4, 13.4, and 20.2 degrees.

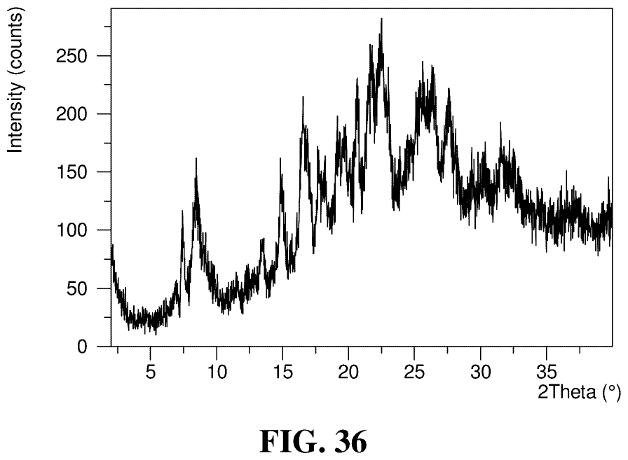

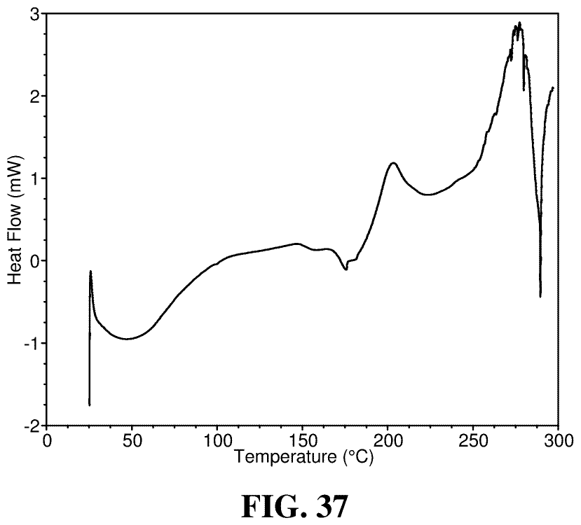

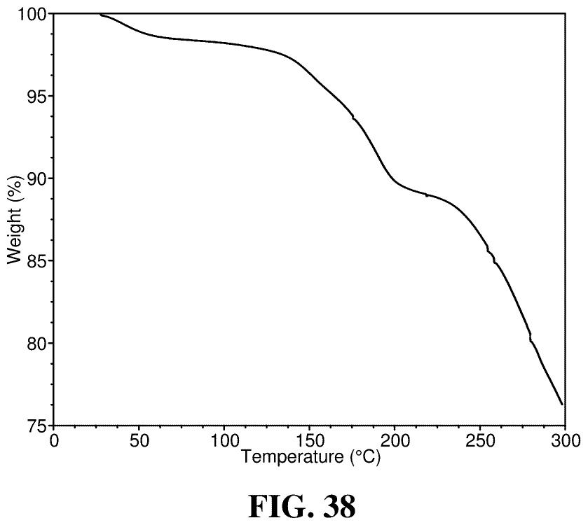

In some embodiments, provided herein is a Formula I Vanillate Form B, wherein the solid form exhibits an X-ray powder diffraction (XRPD) pattern substantially as shown in FIG. 36. Formula I Vanillate Form B may exhibit a differential scanning calorimetry (DSC) thermogram substantially as shown in FIG. 37. Formula I Vanillate Form B may exhibit a thermogravimetric analysis (TGA) thermogram substantially as shown in FIG. 38.

In some embodiments, Formula I Vanillate Form B has at least one, at least two, or at least three of the following properties:
(a) an XRPD pattern substantially as shown in FIG. 36
(b) a DSC thermogram substantially as shown in FIG. 37
(c) a TGA thermogram substantially as shown in FIG. 38.

### Formula I Hippurate

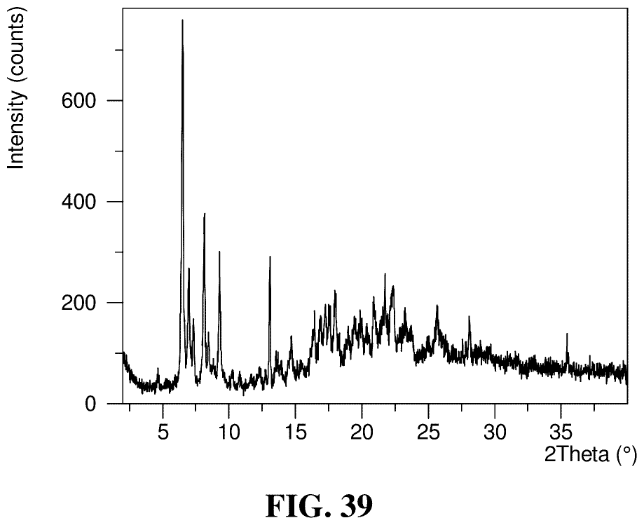

In some embodiments, provided herein is a Formula I Hippurate Form A, wherein the solid form exhibits an X-ray powder diffraction (XRPD) pattern substantially as shown in FIG. 39.

In some embodiments, Formula I Hippurate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 39.

In certain embodiments, Formula I Hippurate Form A has an XRPD pattern comprising degree 2θ-reflections (±0.2 degrees 2θ) at 6.5, 8.2, and 9.3 degrees. In some embodiments, Formula I Hippurate Form A has an XRPD pattern comprising degree 2θ-reflections (±0.2 degrees 2θ) at 6.5, 8.2, and 9.3 degrees and one, two, or three of the degree 2θ-reflections (±0.2 degrees 2θ) at 7.0, 13.1, and 21.8 degrees. In some embodiments, Formula I Hippurate Form A has an XRPD pattern comprising degree 2θ-reflections (±0.2 degrees 2θ) at 6.5, 8.2, and 9.3 degrees and one, two, or three of the degree 2θ-reflections (±0.2 degrees 2θ) at 14.7, 18.0, and 25.7 degrees. In some embodiments, Formula I Hippurate Form A has an XRPD pattern comprising degree 2θ-reflections (±0.2 degrees 2θ) at 6.5, 8.2, 9.3, 7.0, 13.1, 21.8, 14.7, 18.0, and 25.7 degrees.

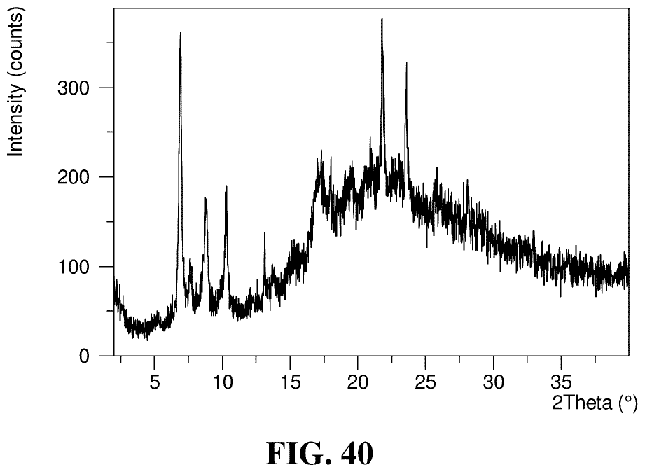

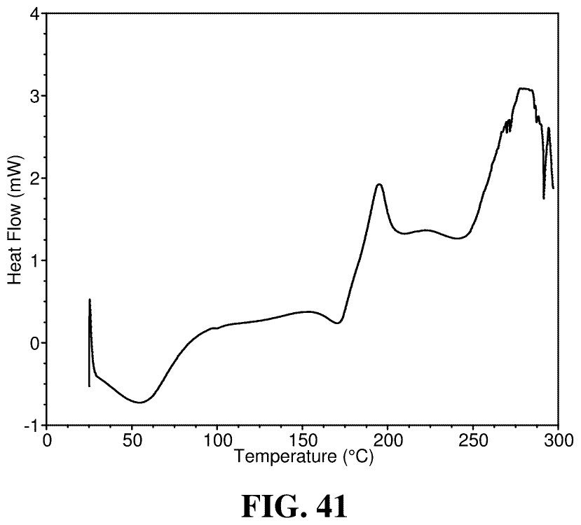

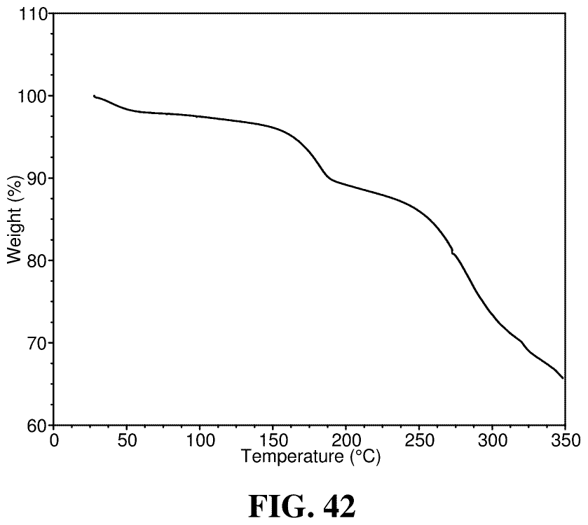

In some embodiments, provided herein is a Formula I Hippurate Form B, wherein the solid form exhibits an X-ray powder diffraction (XRPD) pattern substantially as shown in FIG. 40. Formula I Hippurate Form B may exhibit a differential scanning calorimetry (DSC) thermogram substantially as shown in FIG. 41. Formula I Hippurate Form B may exhibit a thermogravimetric analysis (TGA) thermogram substantially as shown in FIG. 42.

In some embodiments, Formula I Hippurate Form B has at least one, at least two, or at least three of the following properties:
(a) an XRPD pattern substantially as shown in FIG. 40
(b) a DSC thermogram substantially as shown in FIG. 41
(c) a TGA thermogram substantially as shown in FIG. 42.

### Formula I Maleate

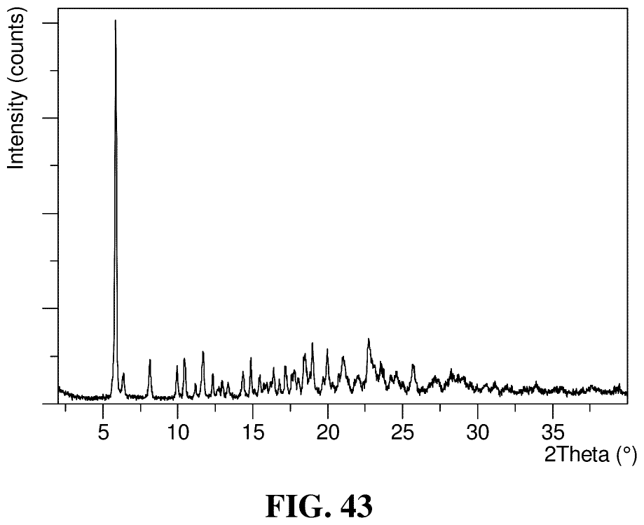

In some embodiments, provided herein is a Formula I Maleate Form A, wherein the solid form exhibits an X-ray powder diffraction (XRPD) pattern substantially as shown in FIG. 43.

In some embodiments, Formula I Maleate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 43.

In certain embodiments, Formula I Maleate Form A has an XRPD pattern comprising degree 2θ-reflections (±0.2 degrees 2θ) at 5.8, 8.2, and 11.7 degrees. In some embodiments, Formula I Maleate Form A has an XRPD pattern comprising degree 2θ-reflections (±0.2 degrees 2θ) at 5.8, 8.2, and 11.7 degrees and one, two, or three of the degree 2θ-reflections (±0.2 degrees 2θ) at 10.0, 10.4, and 14.9 degrees. In some embodiments, Formula I Maleate Form A has an XRPD pattern comprising degree 2θ-reflections (±0.2 degrees 2θ) at 5.8, 8.2, and 11.7 degrees and one, two, or three of the degree 2θ-reflections (±0.2 degrees 2θ) at 6.4, 20.0, and 25.7 degrees. In some embodiments, Formula I Maleate Form A has an XRPD pattern comprising degree 2θ-reflections (±0.2 degrees 2θ) at 5.8, 8.2, 11.7, 10.0, 10.4, 14.9, 6.4, 20.0, and 25.7 degrees.

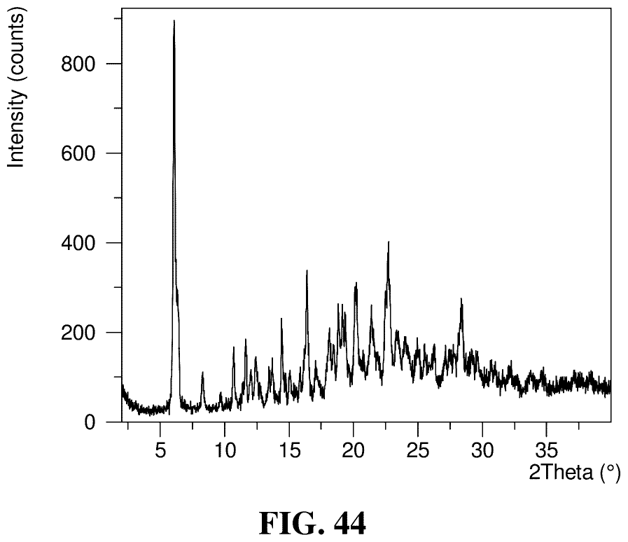

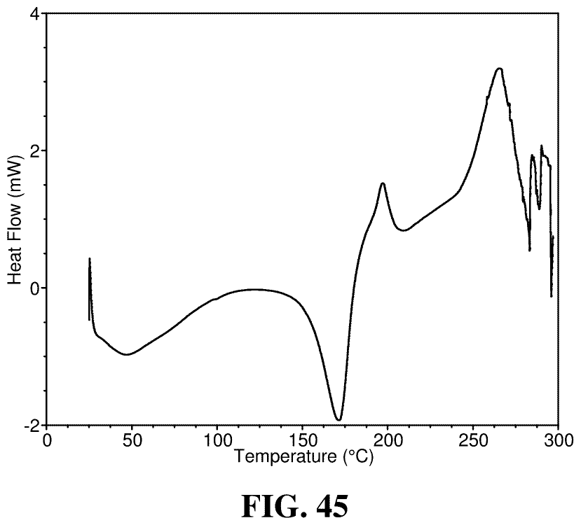

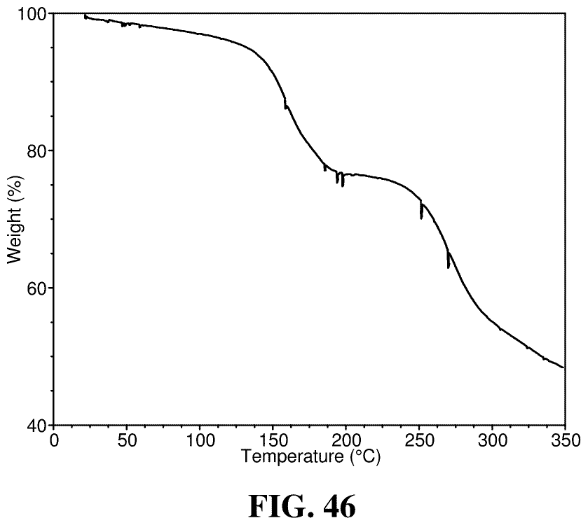

In some embodiments, provided herein is a Formula I Maleate Form B, wherein the solid form exhibits an X-ray powder diffraction (XRPD) pattern substantially as shown in FIG. 44. Formula I Maleate Form B may exhibit a differential scanning calorimetry (DSC) thermogram substantially as shown in FIG. 45. Formula I Maleate Form B may exhibit a thermogravimetric analysis (TGA) thermogram substantially as shown in FIG. 46.

In some embodiments, Formula I Maleate Form B has at least one, at least two, or at least three of the following properties:
(a) an XRPD pattern substantially as shown in FIG. 44
(b) a DSC thermogram substantially as shown in FIG. 45
(c) a TGA thermogram substantially as shown in FIG. 46.

In some embodiments, Formula I Maleate Form B has an XRPD pattern displaying at least two, at least three, at least four, at least five, at least six, at least seven, at least eight, or at least nine of the degree 2θ-reflections with the greatest intensity as the XRPD pattern substantially as shown in FIG. 44.

In certain embodiments, Formula I Maleate Form B has an XRPD pattern comprising degree 2θ-reflections (±0.2 degrees 2θ) at 6.1, 8.3, and 10.7 degrees. In some embodiments, Formula I Maleate Form B has an XRPD pattern comprising degree 2θ-reflections (±0.2 degrees 2θ) at 6.1, 8.3, and 10.7 degrees and one, two, or three of the degree 2θ-reflections (±0.2 degrees 2θ) at 14.4, 16.4, and 20.1 degrees. In some embodiments, Formula I Maleate Form B has an XRPD pattern comprising degree 2θ-reflections (±0.2 degrees 2θ) at 6.1, 8.3, and 10.7 degrees and one, two, or three of the degree 2θ-reflections (±0.2 degrees 2θ) at 21.4, 22.7, and 28.4 degrees. In some embodiments, Formula I Maleate Form B has an XRPD pattern comprising degree 2θ-reflections (±0.2 degrees 2θ) at 6.1, 8.3, 10.7, 14.4, 16.4, 20.1, 21.4, 22.7, and 28.4 degrees.

### Formula I Glyoxylate

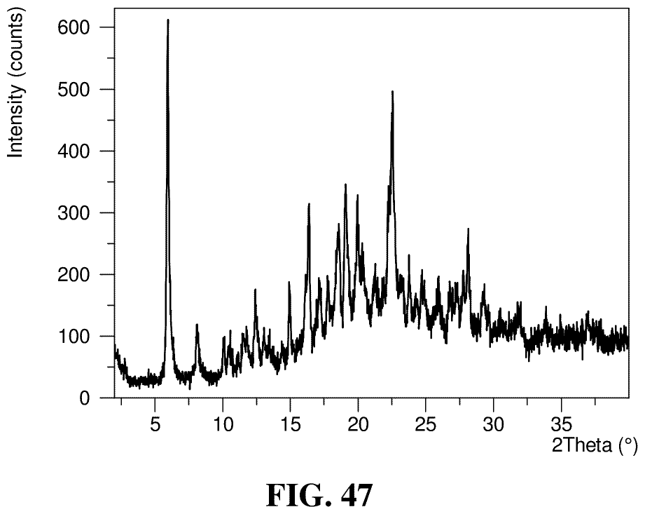

In some embodiments, provided herein is a Formula I Glyoxylate Form A, wherein the solid form exhibits an X-ray powder diffraction (XRPD) pattern substantially as shown in FIG. 47.

In some embodiments, Formula I Glyoxylate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 47.

In certain embodiments, Formula I Glyoxylate Form A has an XRPD pattern comprising degree 2θ-reflections (±0.2 degrees 2θ) at 5.9, 22.5, and 8.1 degrees. In some embodiments, Formula I Glyoxylate Form A has an XRPD pattern comprising degree 2θ-reflections (±0.2 degrees 2θ) at 5.9, 22.5, and 8.1 degrees and one, two, or three of the degree 2θ-reflections (±0.2 degrees 2θ) at 12.4, 16.4, and 19.1 degrees. In some embodiments, Formula I Glyoxylate Form A has an XRPD pattern comprising degree 2θ-reflections (±0.2 degrees 2θ) at 5.9, 22.5, and 8.1 degrees and one, two, or three of the degree 2θ-reflections (±0.2 degrees 2θ) at 15.0, 20.0, and 28.1 degrees. In some embodiments, Formula I Glyoxylate Form A has an XRPD pattern comprising degree 2θ-reflections (±0.2 degrees 2θ) at 5.9, 22.5, 8.1, 12.4, 16.4, 19.1, 15.0, 20.0, and 28.1 degrees.

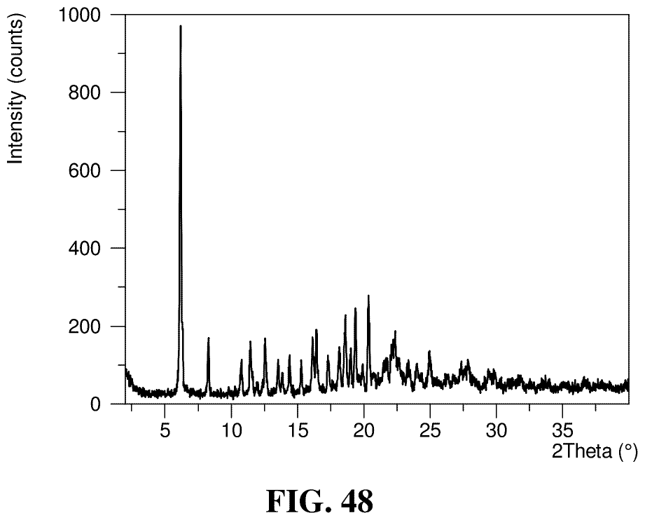

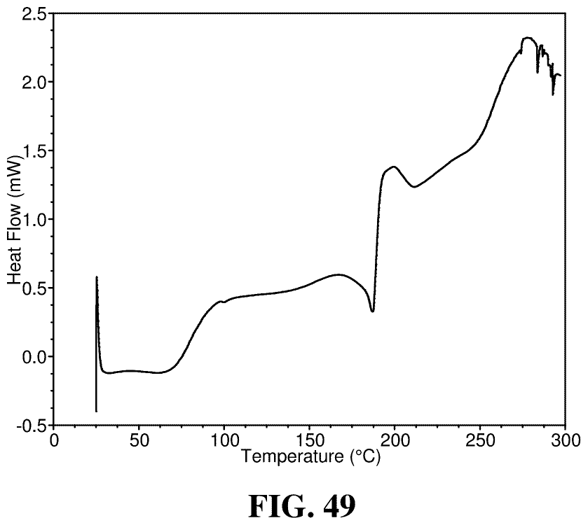

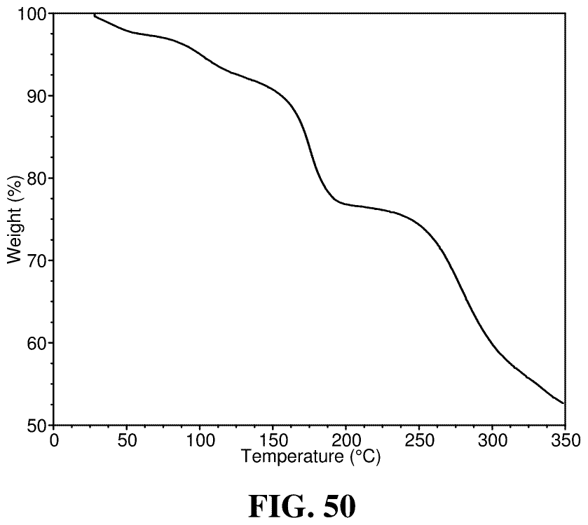

In some embodiments, provided herein is a Formula I Glyoxylate Form B, wherein the solid form exhibits an X-ray powder diffraction (XRPD) pattern substantially as shown in FIG. 48. Formula I Glyoxylate Form B may exhibit a differential scanning calorimetry (DSC) thermogram substantially as shown in FIG. 49. Formula I Glyoxylate Form B may exhibit a thermogravimetric analysis (TGA) thermogram substantially as shown in FIG. 50.

In some embodiments, Formula I Glyoxylate Form B has at least one, at least two, or at least three of the following properties:
(a) an XRPD pattern substantially as shown in FIG. 48
(b) a DSC thermogram substantially as shown in FIG. 49
(c) a TGA thermogram substantially as shown in FIG. 50.

In some embodiments, Formula I Glyoxylate Form B has an XRPD pattern displaying at least two, at least three, at least four, at least five, at least six, at least seven, at least eight, or at least nine of the degree 2θ-reflections with the greatest intensity as the XRPD pattern substantially as shown in FIG. 48.

In certain embodiments, Formula I Glyoxylate Form B has an XRPD pattern comprising degree 2θ-reflections (±0.2 degrees 2θ) at 6.2, 8.3, and 12.6 degrees. In some embodiments, Formula I Glyoxylate Form B has an XRPD pattern comprising degree 2θ-reflections (±0.2 degrees 2θ) at 6.2, 8.3, and 12.6 degrees and one, two, or three of the degree 2θ-reflections (±0.2 degrees 2θ) at 10.8, 16.1, and 16.4 degrees. In some embodiments, Formula I Glyoxylate Form B has an XRPD pattern comprising degree 2θ-reflections (±0.2 degrees 2θ) at 6.2, 8.3, and 12.6 degrees and one, two, or three of the degree 2θ-reflections (±0.2 degrees 2θ) at 18.6, 19.4, and 20.3 degrees. In some embodiments, Formula I Glyoxylate Form B has an XRPD pattern comprising degree 2θ-reflections (±0.2 degrees 2θ) at 6.2, 8.3, 12.6, 10.8, 16.1, 16.4, 18.6, 19.4, and 20.3 degrees.

### Formula I L-Pyroglutamate

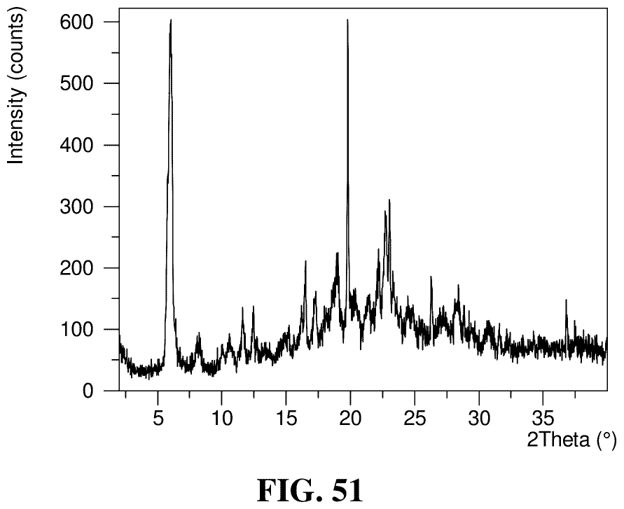

In some embodiments, provided herein is a solid form of Formula I L-Pyroglutamate wherein the solid form exhibits an X-ray powder diffraction (XRPD) pattern substantially as shown in FIG. 51.

In some embodiments, Formula I L-Pyroglutamate has an XRPD pattern displaying at least two, at least three, at least four, at least five, at least six, at least seven, at least eight, or at least nine of the degree 2θ-reflections with the greatest intensity as the XRPD pattern substantially as shown in FIG. 51.

In certain embodiments, Formula I L-Pyroglutamate has an XRPD pattern comprising degree 2θ-reflections (±0.2 degrees 2θ) at 6.1, 16.5, and 19.8 degrees. In some embodiments, Formula I L-Pyroglutamate has an XRPD pattern comprising degree 2θ-reflections (±0.2 degrees 2θ) at 6.1, 16.5, and 19.8 degrees and one, two, or three of the degree 2θ-reflections (±0.2 degrees 2θ) at 11.6, 12.4, and 17.3 degrees. In some embodiments, Formula I L-Pyroglutamate has an XRPD pattern comprising degree 2θ-reflections (±0.2 degrees 2θ) at 6.1, 16.5, and 19.8 degrees and one, two, or three of the degree 2θ-reflections (±0.2 degrees 2θ) at 22.8, 23.0, and 28.4 degrees. In some embodiments, Formula I L-Pyroglutamate has an XRPD pattern comprising degree 2θ-reflections (±0.2 degrees 2θ) at 6.1, 16.5, 19.8, 11.6, 12.4, 17.3, 22.8, 23.0, and 28.4 degrees.

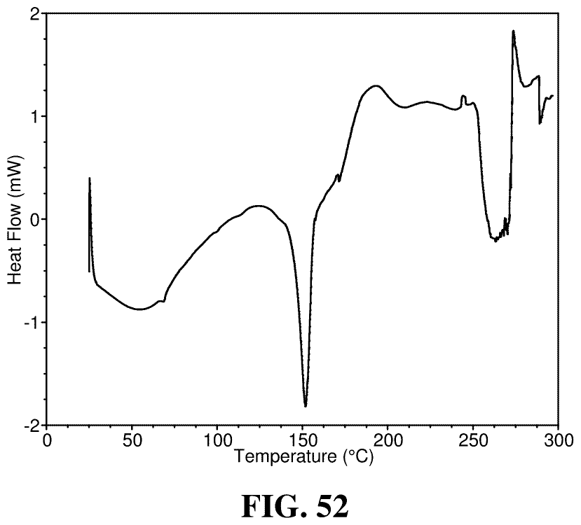

In some embodiments, provided herein is a solid form of Formula I L-Pyroglutamate, wherein the solid form exhibits a differential scanning calorimetry (DSC) thermogram substantially as shown in FIG. 52.

### Formula I 2-Naphthalene Sulfonate

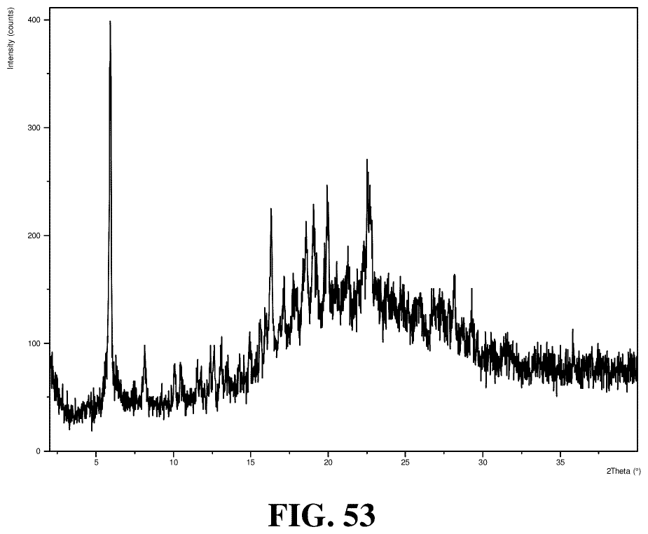

In some embodiments, provided herein is a Formula I 2-Naphthalene Sulfonate, wherein the solid form exhibits an X-ray powder diffraction (XRPD) pattern substantially as shown in FIG. 53.

In some embodiments, Formula I 2-Naphthalene Sulfonate has an XRPD pattern displaying at least two, at least three, at least four, at least five, at least six, at least seven, at least eight, or at least nine of the degree 2θ-reflections with the greatest intensity as the XRPD pattern substantially as shown in FIG. 53.

In certain embodiments, Formula I 2-Naphthalene Sulfonate has an XRPD pattern comprising degree 2θ-reflections (±0.2 degrees 2θ) at 5.9, 8.1, and 16.3 degrees. In some embodiments, Formula I 2-Naphthalene Sulfonate Form A has an XRPD pattern comprising degree 2θ-reflections (±0.2 degrees 2θ) at 5.9, 8.1, and 16.3 degrees and one, two, or three of the degree 2θ-reflections (±0.2 degrees 2θ) at 18.6, 19.1, and 20.0 degrees. In some embodiments, Formula I 2-Naphthalene Sulfonate Form A has an XRPD pattern comprising degree 2θ-reflections (±0.2 degrees 2θ) at 5.9, 8.1, and 16.3 degrees and one, two, or three of the degree 2θ-reflections (±0.2 degrees 2θ) at 10.1, 12.6, and 13.1 degrees. In some embodiments, Formula I 2-Naphthalene Sulfonate Form A has an XRPD pattern comprising degree 2θ-reflections (±0.2 degrees 2θ) at 5.9, 8.1, 16.3, 18.6, 19.1, 20.0, 10.1, 12.6, and 13.1 degrees.

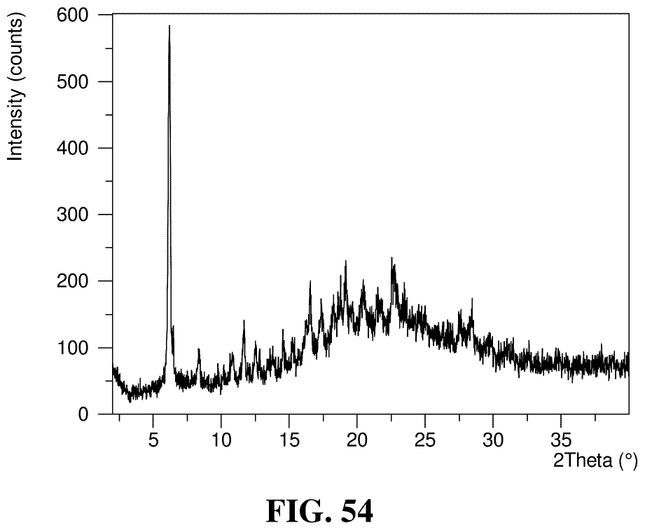

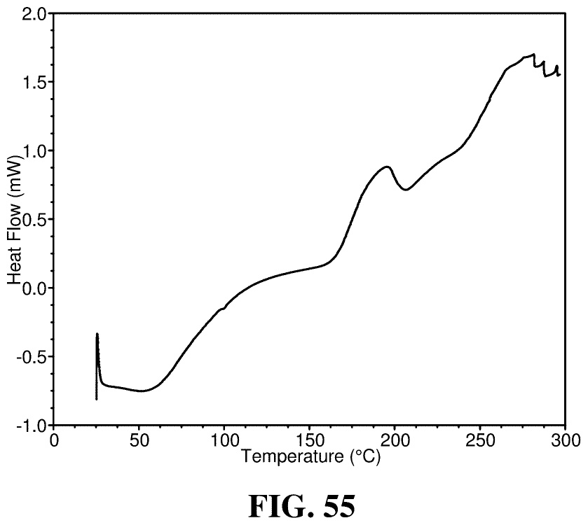

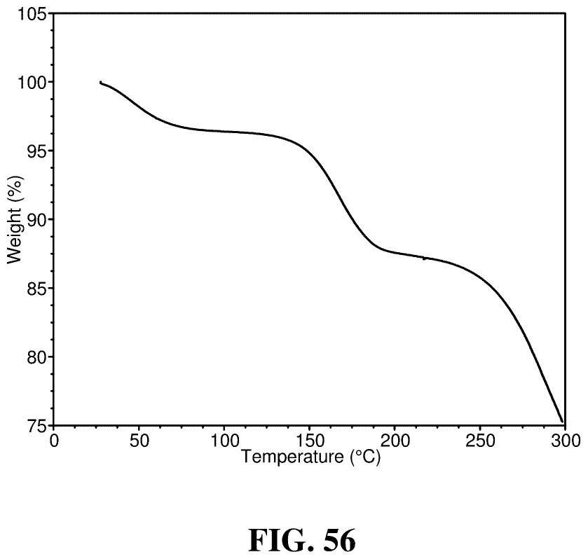

In some embodiments, provided herein is air-dried Formula I 2-Naphthalene Sulfonate, wherein the solid form exhibits an X-ray powder diffraction (XRPD) pattern substantially as shown in FIG. 54. Formula I 2-Naphthalene Sulfonate may exhibit a differential scanning calorimetry (DSC) thermogram substantially as shown in FIG. 55. Formula I 2-Naphthalene Sulfonate may exhibit a thermogravimetric analysis (TGA) thermogram substantially as shown in FIG. 56.

In some embodiments, Formula I 2-Naphthalene Sulfonate has at least one, at least two, or at least three of the following properties:
(a) an XRPD pattern substantially as shown in FIG. 54
(b) a DSC thermogram substantially as shown in FIG. 55
(c) a TGA thermogram substantially as shown in FIG. 56.

In some embodiments, Formula I 2-Naphthalene Sulfonate has an XRPD pattern displaying at least two, at least three, at least four, at least five, at least six, at least seven, at least eight, or at least nine of the degree 2θ-reflections with the greatest intensity as the XRPD pattern substantially as shown in FIG. 54.

In certain embodiments, Formula I 2-Naphthalene Sulfonate has an XRPD pattern comprising degree 2θ-reflections (±0.2 degrees 2θ) at 6.2, 8.4, and 16.5 degrees. In some embodiments, Formula I 2-Naphthalene Sulfonate has an XRPD pattern comprising degree 2θ-reflections (±0.2 degrees 2θ) at 6.2, 8.4, and 16.5 degrees and one, two, or three of the degree 2θ-reflections (±0.2 degrees 2θ) at 10.8, 11.7, and 19.1 degrees. In some embodiments, Formula I 2-Naphthalene Sulfonate has an XRPD pattern comprising degree 2θ-reflections (±0.2 degrees 2θ) at 6.2, 8.4, and 16.5 degrees and one, two, or three of the degree 2θ-reflections (±0.2 degrees 2θ) at 12.6, 14.6, and 22.6 degrees. In some embodiments, Formula I 2-Naphthalene Sulfonate has an XRPD pattern comprising degree 2θ-reflections (±0.2 degrees 2θ) at 6.2, 8.4, 16.5, 10.8, 11.7, 19.1, 12.6, 14.6, and 22.6 degrees.

### Formula I 1-Naphthalene Sulfonate

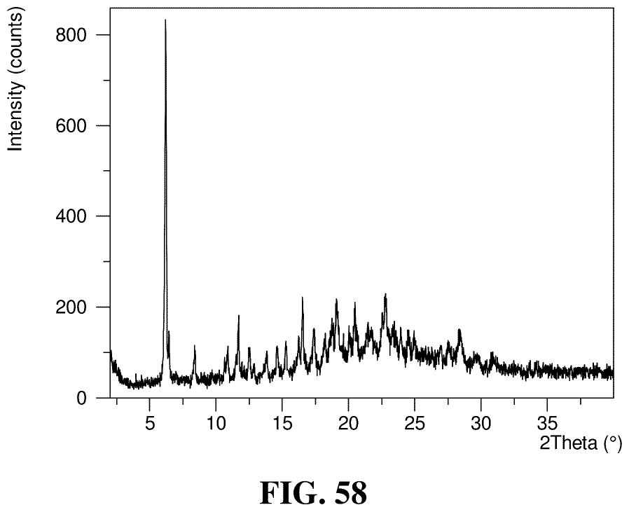

In some embodiments, provided herein is a Formula I 1-Naphthalene Sulfonate wet cake, wherein the solid form exhibits an X-ray powder diffraction (XRPD) pattern substantially as shown in FIG. 58.

In some embodiments, Formula I 1-Naphthalene Sulfonate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 58.

In certain embodiments, Formula I 1-Naphthalene Sulfonate wet cake has an XRPD pattern comprising degree 2θ-reflections (±0.2 degrees 2θ) at 5.9, 8.1, and 16.3 degrees. In some embodiments, Formula I 1-Naphthalene Sulfonate wet cake has an XRPD pattern comprising degree 2θ-reflections (±0.2 degrees 2θ) at 5.9, 8.1, and 16.3 degrees and one, two, or three of the degree 2θ-reflections (±0.2 degrees 2θ) at 10.0, 10.5, and 20.0 degrees. In some embodiments, Formula I 1-Naphthalene Sulfonate wet cake has an XRPD pattern comprising degree 2θ-reflections (±0.2 degrees 2θ) at 5.9, 8.1, and 16.3 degrees and one, two, or three of the degree 2θ-reflections (±0.2 degrees 2θ) at 18.6, 19.0, and 22.6 degrees. In some embodiments, Formula I 1-Naphthalene Sulfonate wet cake has an XRPD pattern comprising degree 2θ-reflections (±0.2 degrees 2θ) at 5.9, 8.1, 16.3, 10.0, 10.5, 20.0, 18.6, 19.0, and 22.6 degrees.

In some embodiments, provided herein is a solid form of Formula I 1-Naphthalene Sulfonate, wherein the solid form exhibits an X-ray powder diffraction (XRPD) pattern substantially as shown in FIG. 58. The solid form of Formula I 1-Naphthalene Sulfonate may exhibit a differential scanning calorimetry (DSC) thermogram substantially as shown in FIG. 58.

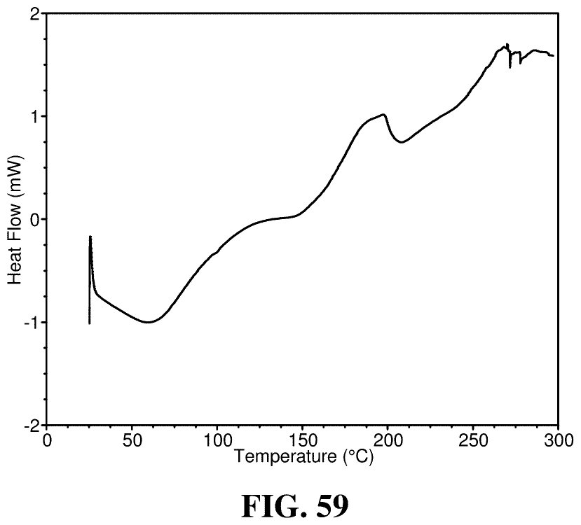

In some embodiments, a solid form of Formula I 1-Naphthalene Sulfonate has one or both of the following properties:
(a) an XRPD pattern substantially as shown in FIG. 58
(b) a DSC thermogram substantially as shown in FIG. 59.

In some embodiments, Formula I 1-Naphthalene Sulfonate has an XRPD pattern displaying at least two, at least three, at least four, at least five, at least six, at least seven, at least eight, or at least nine of the degree 2θ-reflections with the greatest intensity as the XRPD pattern substantially as shown in FIG. 58.

In certain embodiments, Formula I 1-Naphthalene Sulfonate has an XRPD pattern comprising degree 2θ-reflections (±0.2 degrees 2θ) at 6.2, 8.4, and 16.5 degrees. In some embodiments, Formula I 1-Naphthalene Sulfonate has an XRPD pattern comprising degree 2θ-reflections (±0.2 degrees 2θ) at 6.2, 8.4, and 16.5 degrees and one, two, or three of the degree 2θ-reflections (±0.2 degrees 2θ) at 10.9, 11.7, and 20.5 degrees. In some embodiments, Formula I 1-Naphthalene Sulfonate has an XRPD pattern comprising degree 2θ-reflections (±0.2 degrees 2θ) at 6.2, 8.4, and 16.5 degrees and one, two, or three of the degree 2θ-reflections (±0.2 degrees 2θ) at 14.6, 15.3, and 17.3 degrees. In some embodiments, Formula I 1-Naphthalene Sulfonate has an XRPD pattern comprising degree 2θ-reflections (±0.2 degrees 2θ) at 6.2, 8.4, 16.5, 10.9, 11.7, 20.5, 14.6, 15.3, and 17.3 degrees.

### Formula I 1-Hydroxy-2-Naphthoate

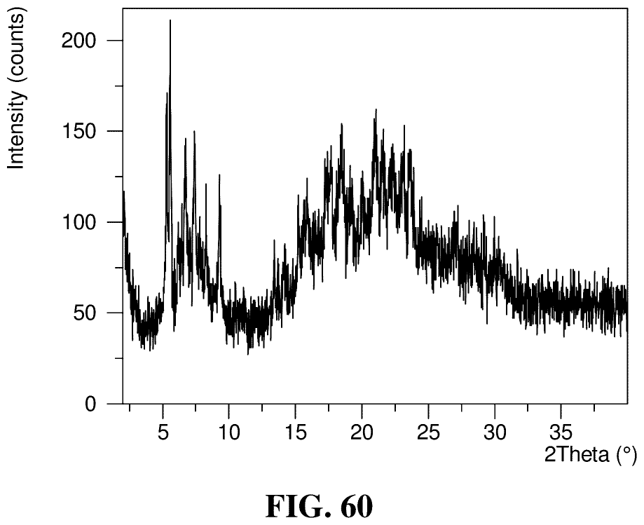

In some embodiments, provided herein is a Formula I 1-Hydroxy-2-Naphthoate wet cake, wherein the Formula I 1-Hydroxy-2-Naphthoate wet cake exhibits an X-ray powder diffraction (XRPD) pattern substantially as shown in FIG. 60.

In some embodiments, Formula I 1-Hydroxy-2-Naphthoate has an XRPD pattern displaying at least two, at least three, at least four, at least five, at least six, at least seven, at least eight, or at least nine of the degree 2θ-reflections with the greatest intensity as the XRPD pattern substantially as shown in FIG. 60.

In certain embodiments, Formula I 1-Hydroxy-2-Naphthoate wet cake has an XRPD pattern comprising degree 2θ-reflections (±0.2 degrees 2θ) at 5.3, 5.6, and 7.4 degrees. In some embodiments, Formula I 1-Hydroxy-2-Naphthoate wet cake has an XRPD pattern comprising degree 2θ-reflections (±0.2 degrees 2θ) at 5.3, 5.6, and 7.4 degrees and one, two, or three of the degree 2θ-reflections (±0.2 degrees 2θ) at 6.7, 9.3, and 21.0 degrees. In some embodiments, Formula I 1-Hydroxy-2-Naphthoate wet cake has an XRPD pattern comprising degree 2θ-reflections (±0.2 degrees 2θ) at 5.3, 5.6, and 7.4 degrees and one, two, or three of the degree 2θ-reflections (±0.2 degrees 2θ) at 17.5, 18.5, and 23.6 degrees. In some embodiments, Formula I 1-Hydroxy-2-Naphthoate wet cake has an XRPD pattern comprising degree 2θ-reflections (±0.2 degrees 2θ) at 5.3, 5.6, 7.4, 6.7, 9.3, 21.0, 17.5, 18.5, and 23.6 degrees.

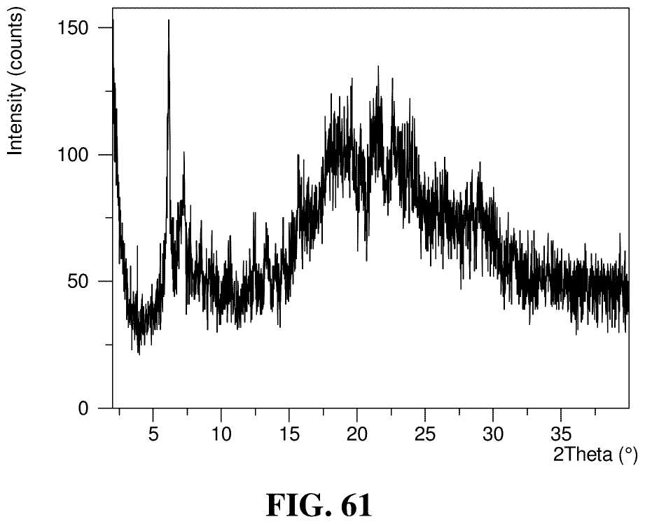

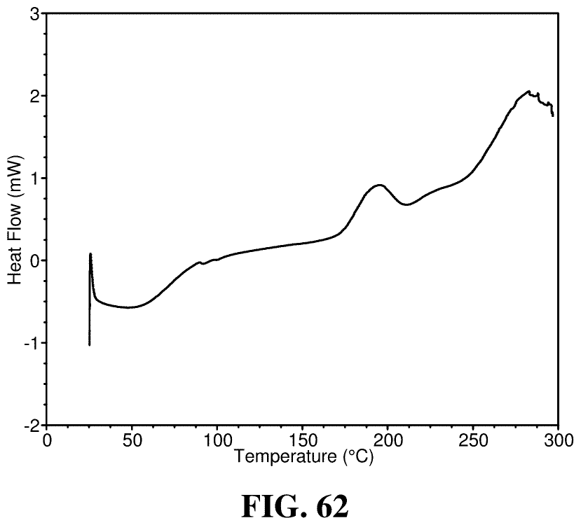

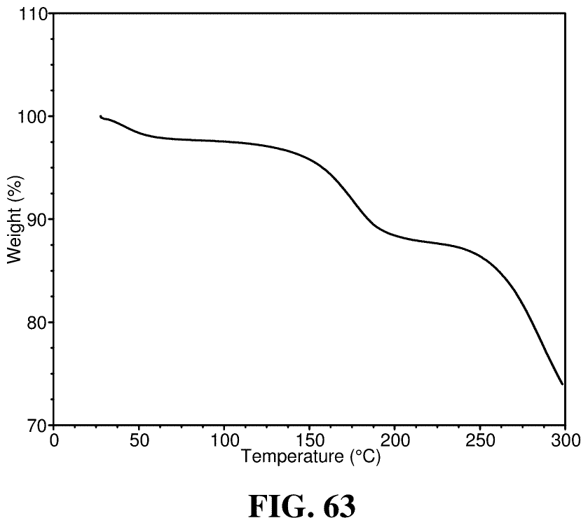

In some embodiments, provided herein is a solid form of Formula I 1-Hydroxy-2-Naphthoate, wherein the solid form exhibits an X-ray powder diffraction (XRPD) pattern substantially as shown in FIG. 61. Formula I 1-Hydroxy-2-Naphthoate may exhibit a differential scanning calorimetry (DSC) thermogram substantially as shown in FIG. 62. Formula I 1-Hydroxy-2-Naphthoate may exhibit a thermogravimetric analysis (TGA) thermogram substantially as shown in FIG. 63.

In some embodiments, a solid form of Formula I 1-Hydroxy-2-Naphthoate has at least one, at least two, or at least three of the following properties:
(a) an XRPD pattern substantially as shown in FIG. 61
(b) a DSC thermogram substantially as shown in FIG. 62
(c) a TGA thermogram substantially as shown in FIG. 63.

In some embodiments, a solid form of Formula I 1-Hydroxy-2-Naphthoate has an XRPD pattern displaying at least two, or at least three of the degree 2θ-reflections with the greatest intensity as the XRPD pattern substantially as shown in FIG. 61.

In certain embodiments, a solid form of Formula I 1-Hydroxy-2-Naphthoate has an XRPD pattern comprising degree 2θ-reflections (±0.2 degrees 2θ) at 6.1, 7.3, and 15.9 degrees.

### Formula I S-Mandelate

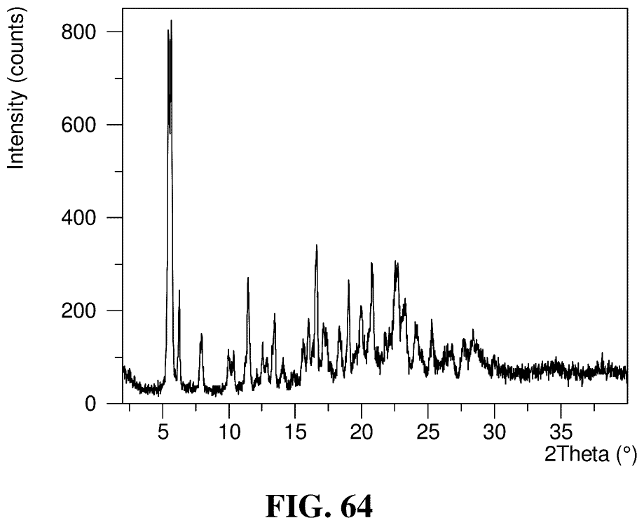

In some embodiments, provided herein is a Formula I S-Mandelate Form A, wherein the solid form exhibits an X-ray powder diffraction (XRPD) pattern substantially as shown in FIG. 64.

In some embodiments, Formula I S-Mandelate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 64.

In certain embodiments, Formula I S-Mandelate Form A has an XRPD pattern comprising degree 2θ-reflections (±0.2 degrees 2θ) at 5.5, 5.7, and 6.3 degrees. In some embodiments, Formula I S-Mandelate Form A has an XRPD pattern comprising degree 2θ-reflections (±0.2 degrees 2θ) at 5.5, 5.7, and 6.3 degrees and one, two, or three of the degree 2θ-reflections (±0.2 degrees 2θ) at 11.4, 16.6, and 20.8 degrees. In some embodiments, Formula I S-Mandelate Form A has an XRPD pattern comprising degree 2θ-reflections (±0.2 degrees 2θ) at 5.5, 5.7, and 6.3 degrees and one, two, or three of the degree 2θ-reflections (±0.2 degrees 2θ) at 10.0, 10.4, and 19.0 degrees. In some embodiments, Formula I S-Mandelate Form A has an XRPD pattern comprising degree 2θ-reflections (±0.2 degrees 2θ) at 5.5, 5.7, 6.3, 11.4, 16.6, 20.8, 10.0, 10.4, and 19.0 degrees.

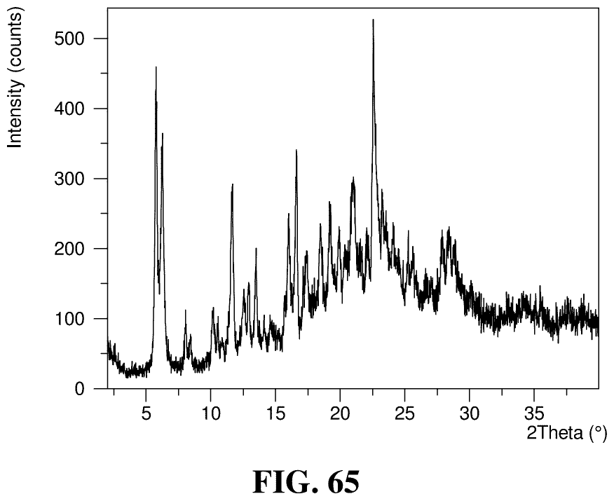

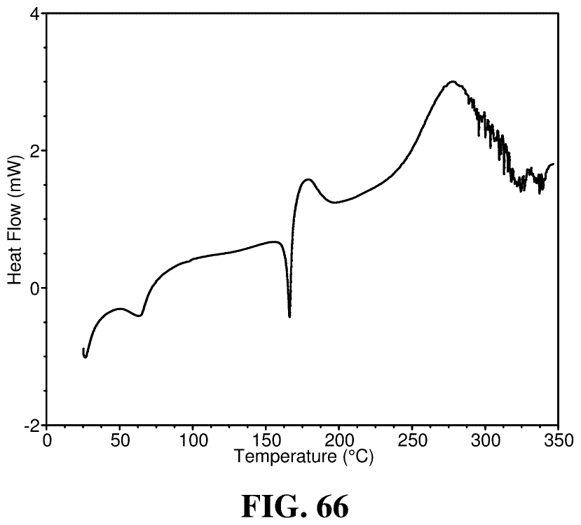

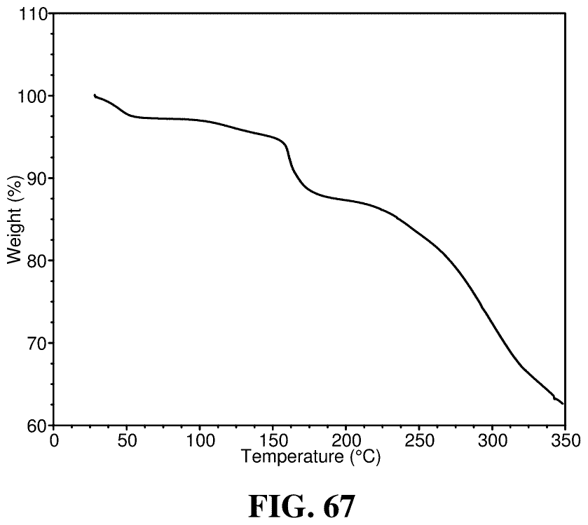

In some embodiments, provided herein is a Formula I S-Mandelate Form B, wherein the solid form exhibits an X-ray powder diffraction (XRPD) pattern substantially as shown in FIG. 65. Formula I S-Mandelate Form B may exhibit a differential scanning calorimetry (DSC) thermogram substantially as shown in FIG. 66. Formula I S-Mandelate Form B may exhibit a thermogravimetric analysis (TGA) thermogram substantially as shown in FIG. 67.

In some embodiments, Formula I S-Mandelate Form B has at least one, at least two, or at least three of the following properties:
(a) an XRPD pattern substantially as shown in FIG. 65
(b) a DSC thermogram substantially as shown in FIG. 66
(c) a TGA thermogram substantially as shown in FIG. 67.

In some embodiments, Formula I S-Mandelate Form B has an XRPD pattern displaying at least two, at least three, at least four, at least five, at least six, at least seven, at least eight, or at least nine of the degree 2θ-reflections with the greatest intensity as the XRPD pattern substantially as shown in FIG. 65.

In certain embodiments, Formula I S-Mandelate Form B has an XRPD pattern comprising degree 2θ-reflections (±0.2 degrees 2θ) at 5.7, 6.2, and 22.6 degrees. In some embodiments, Formula I S-Mandelate Form B has an XRPD pattern comprising degree 2θ-reflections (±0.2 degrees 2θ) at 5.7, 6.2, and 22.6 degrees and one, two, or three of the degree 2θ-reflections (±0.2 degrees 2θ) at 8.0, 8.4, and 11.7 degrees. In some embodiments, Formula I S-Mandelate Form B has an XRPD pattern comprising degree 2θ-reflections (±0.2 degrees 2θ) at 5.7, 6.2, and 22.6 degrees and one, two, or three of the degree 2θ-reflections (±0.2 degrees 2θ) at 13.5, 16.0, and 16.6 degrees. In some embodiments, Formula I S-Mandelate Form B has an XRPD pattern comprising degree 2θ-reflections (±0.2 degrees 2θ) at 5.7, 6.2, 22.6, 8.0, 8.4, 11.7, 13.5, 16.0, and 16.6 degrees.

In some embodiments, Formula I S-Mandelate Form B has a differential scanning calorimetry thermogram comprising an endotherm with onset at about 50° C.

### Formula I Gentisate

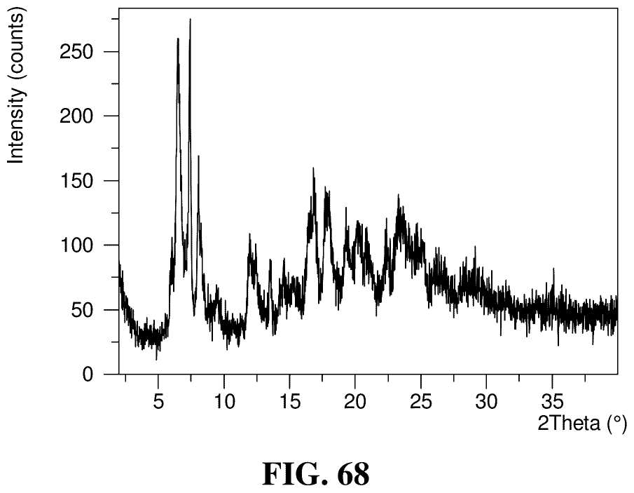

In some embodiments, provided herein is a Formula I Gentisate wet cake, wherein the Formula I Gentisate wet cake exhibits an X-ray powder diffraction (XRPD) pattern substantially as shown in FIG. 68.

In some embodiments, Formula I Gentisate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 68.

In certain embodiments, Formula I Gentisate Form A has an XRPD pattern comprising degree 2θ-reflections (±0.2 degrees 2θ) at 7.4, 6.5, and 8.0 degrees. In some embodiments, Formula I Gentisate wet cake has an XRPD pattern comprising degree 2θ-reflections (±0.2 degrees 2θ) at 7.4, 6.5, and 8.0 degrees and one, two, or three of the degree 2θ-reflections (±0.2 degrees 2θ) at 13.5, 16.9, and 12.0 degrees. In some embodiments, Formula I Gentisate wet cake has an XRPD pattern comprising degree 2θ-reflections (±0.2 degrees 2θ) at 7.4, 6.5, and 8.0 degrees and one, two, or three of the degree 2θ-reflections (±0.2 degrees 2θ) at 25.1, 22.4, and 19.3 degrees. In some embodiments, Formula I Gentisate wet cake has an XRPD pattern comprising degree 2θ-reflections (±0.2 degrees 2θ) at 7.4, 6.5, 8.0, 13.5, 16.9, 12.0, 25.1, 22.4, and 19.3 degrees.

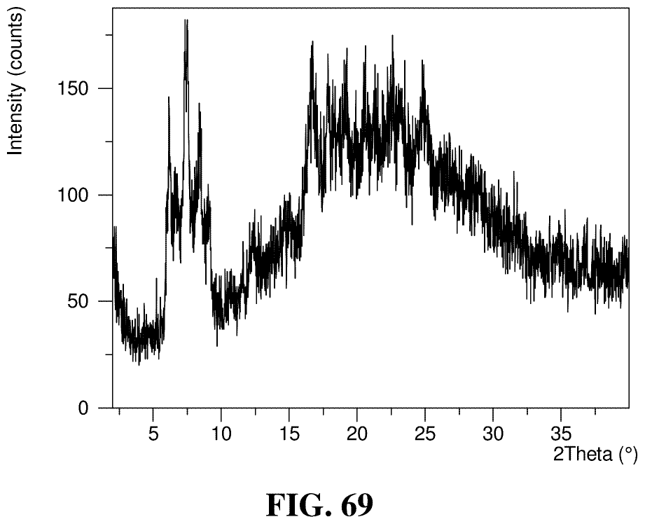

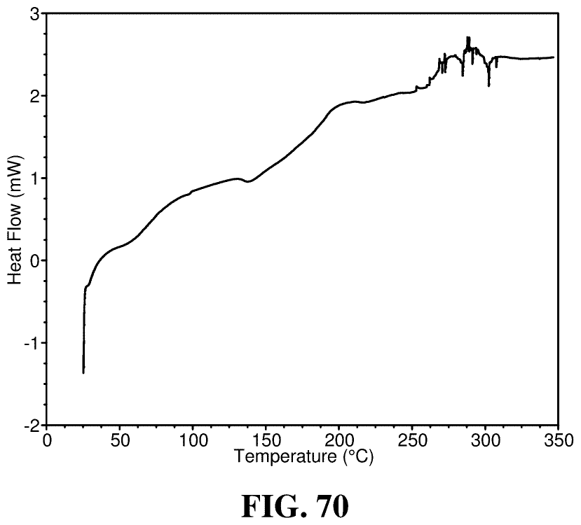

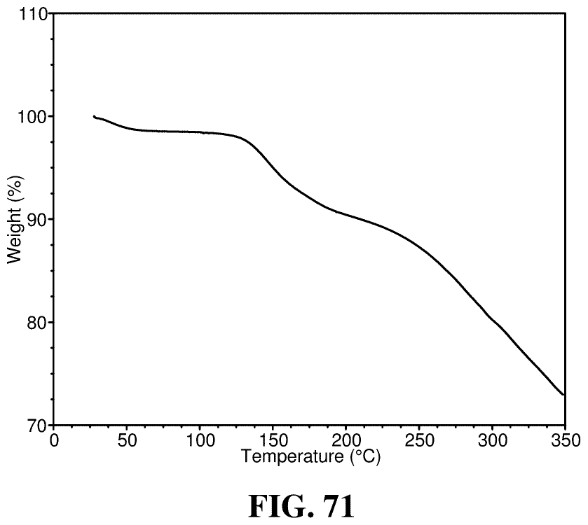

In some embodiments, provided herein is a solid form of Formula I Gentisate, wherein the solid form exhibits an X-ray powder diffraction (XRPD) pattern substantially as shown in FIG. 69. Formula I Gentisate may exhibit a differential scanning calorimetry (DSC) thermogram substantially as shown in FIG. 70. Formula I Gentisate may exhibit a thermogravimetric analysis (TGA) thermogram substantially as shown in FIG. 71.

In some embodiments, a solid form of Formula I Gentisate has at least one, at least two, or at least three of the following properties:
(a) an XRPD pattern substantially as shown in FIG. 69
(b) a DSC thermogram substantially as shown in FIG. 70
(c) a TGA thermogram substantially as shown in FIG. 71.

In some embodiments, a solid form of Formula I Gentisate has an XRPD pattern displaying at least two, at least three, at least four, at least five, at least six, at least seven, at least eight, or at least nine of the degree 2θ-reflections with the greatest intensity as the XRPD pattern substantially as shown in FIG. 69.

In certain embodiments, a solid form of Formula I Gentisate has an XRPD pattern comprising degree 2θ-reflections (±0.2 degrees 2θ) at 7.4, 6.1, and 8.4 degrees. In some embodiments, Formula I Gentisate has an XRPD pattern comprising degree 2θ-reflections (±0.2 degrees 2θ) at 7.4, 6.1, and 8.4 degrees and one, two, or three of the degree 2θ-reflections (±0.2 degrees 2θ) at 16.7, 17.9, and 9.1 degrees. In some embodiments, Formula I Gentisate has an XRPD pattern comprising degree 2θ-reflections (±0.2 degrees 2θ) at 7.4, 6.1, and 8.4 degrees and one, two, or three of the degree 2θ-reflections (±0.2 degrees 2θ) at 24.9, 22.8, and 12.3 degrees. In some embodiments, Formula I Gentisate has an XRPD pattern comprising degree 2θ-reflections (±0.2 degrees 2θ) at 7.4, 6.1, 8.4, 16.7, 17.9, 9.1, 24.9, 22.8, and 12.3 degrees.

### Formula I Citrate

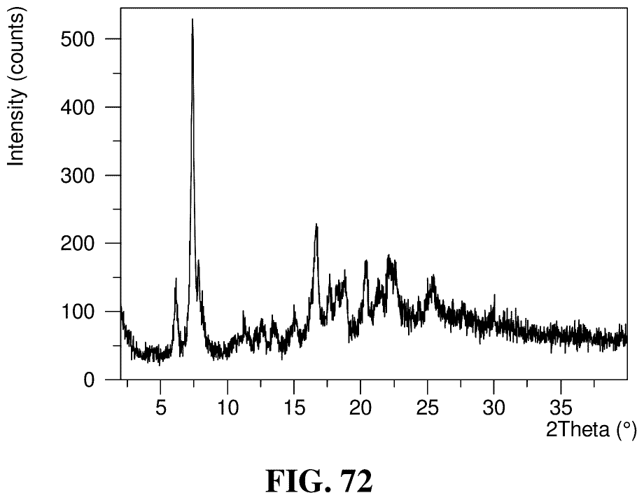

In some embodiments, provided herein is a Formula I Citrate wet cake, wherein the Formula I Citrate wet cake exhibits an X-ray powder diffraction (XRPD) pattern substantially as shown in FIG. 72.

In some embodiments, Formula I Citrate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 72.

In certain embodiments, Formula I Citrate wet cake has an XRPD pattern comprising degree 2θ-reflections (±0.2 degrees 2θ) at 7.4, 7.9, and 16.7 degrees. In some embodiments, Formula I Citrate wet cake has an XRPD pattern comprising degree 2θ-reflections (±0.2 degrees 2θ) at 7.4, 7.9, and 16.7 degrees and one, two, or three of the degree 2θ-reflections (±0.2 degrees 2θ) at 15.1, 17.7, and 20.4 degrees. In some embodiments, Formula I Citrate wet cake has an XRPD pattern comprising degree 2θ-reflections (±0.2 degrees 2θ) at 7.4, 7.9, and 16.7 degrees and one, two, or three of the degree 2θ-reflections (±0.2 degrees 2θ) at 11.4, 18.9, and 22.1 degrees. In some embodiments, Formula I Citrate wet cake has an XRPD pattern comprising degree 2θ-reflections (±0.2 degrees 2θ) at 7.4, 7.9, 16.7, 15.1, 17.7, 20.4, 11.4, 18.9, and 22.1 degrees.

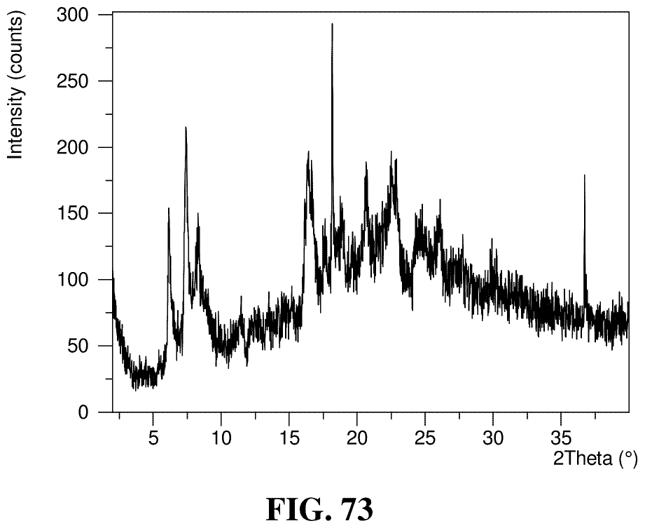

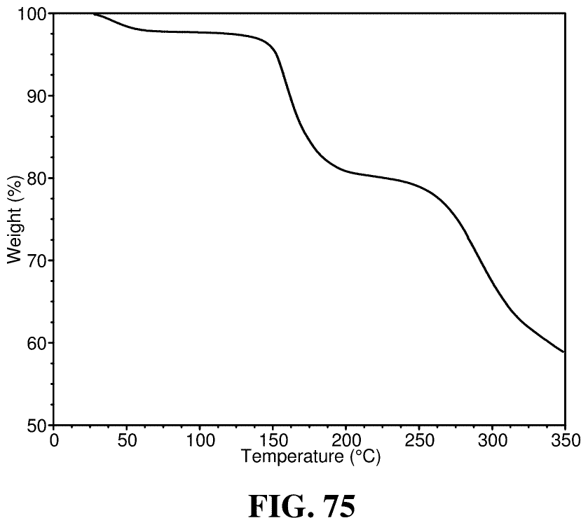

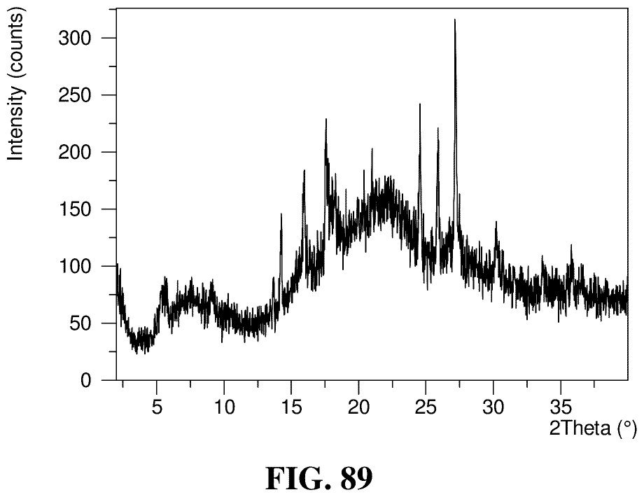

In some embodiments, provided herein is a solid form of Formula I Citrate, wherein the solid form exhibits an X-ray powder diffraction (XRPD) pattern substantially as shown in FIG. 89. Formula I Citrate may exhibit a differential scanning calorimetry (DSC) thermogram substantially as shown in FIG. 73. Formula I Citrate may exhibit a thermogravimetric analysis (TGA) thermogram substantially as shown in FIG. 75.

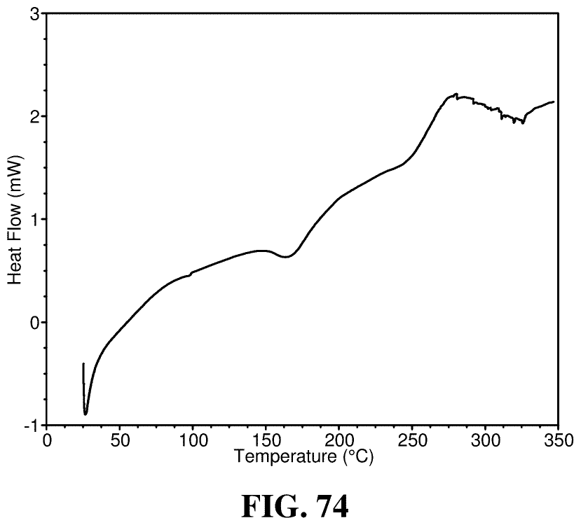

In some embodiments, Formula I Citrate Form B has at least one, at least two, or all of the following properties:
(a) an XRPD pattern substantially as shown in FIG. 73
(b) a DSC thermogram substantially as shown in FIG. 74
(c) a TGA thermogram substantially as shown in FIG. 75.

In some embodiments, Formula I Citrate has an XRPD pattern displaying at least two, at least three, at least four, at least five, at least six, at least seven, at least eight, or at least nine of the degree 2θ-reflections with the greatest intensity as the XRPD pattern substantially as shown in FIG. 73.

In certain embodiments, Formula I Citrate has an XRPD pattern comprising degree 2θ-reflections (±0.2 degrees 2θ) at 6.1, 7.4, and 18.2 degrees. In some embodiments, Formula I Citrate has an XRPD pattern comprising degree 2θ-reflections (±0.2 degrees 2θ) at 6.1, 7.4, and 18.2 degrees and one, two, or three of the degree 2θ-reflections (±0.2 degrees 2θ) at 8.3, 16.4, and 36.7 degrees. In some embodiments, Formula I Citrate has an XRPD pattern comprising degree 2θ-reflections (±0.2 degrees 2θ) at 6.1, 7.4, and 18.2 degrees and one, two, or three of the degree 2θ-reflections (±0.2 degrees 2θ) at 20.7, 24.5, and 26.0 degrees. In some embodiments, Formula I Citrate has an XRPD pattern comprising degree 2θ-reflections (±0.2 degrees 2θ) at 6.1, 7.4, 18.2, 8.3, 16.4, 36.7, 20.7, 24.5, and 26.0 degrees.

### Formula I R-Mandelate

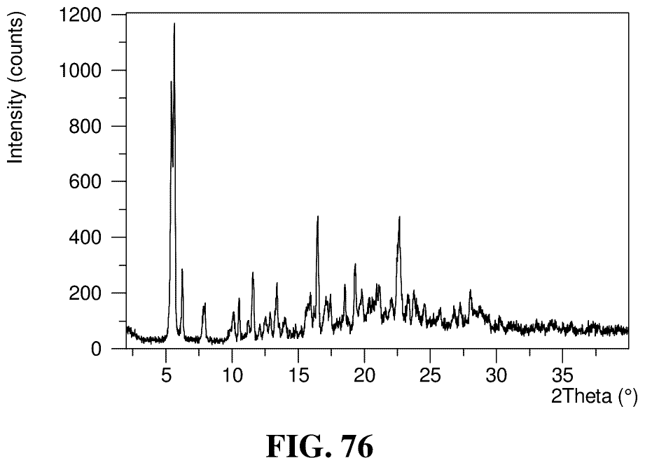

In some embodiments, provided herein is a Formula I R-Mandelate Form A, wherein the solid form exhibits an X-ray powder diffraction (XRPD) pattern substantially as shown in FIG. 76.

In some embodiments, Formula I R-Mandelate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 76.

In certain embodiments, Formula I R-Mandelate Form A has an XRPD pattern comprising degree 2θ-reflections (±0.2 degrees 2θ) at 5.7, 5.4, and 16.5 degrees. In some embodiments, Formula I R-Mandelate Form A has an XRPD pattern comprising degree 2θ-reflections (±0.2 degrees 2θ) at 5.7, 5.4, and 16.5 degrees and one, two, or three of the degree 2θ-reflections (±0.2 degrees 2θ) at 6.2, 7.8, and 11.5 degrees. In some embodiments, Formula I R-Mandelate Form A has an XRPD pattern comprising degree 2θ-reflections (±0.2 degrees 2θ) at 5.7, 5.4, and 16.5 degrees and one, two, or three of the degree 2θ-reflections (±0.2 degrees 2θ) at 10.5, 13.4, and 18.5 degrees. In some embodiments, Formula I R-Mandelate Form A has an XRPD pattern comprising degree 2θ-reflections (±0.2 degrees 2θ) at 5.7, 5.4, 16.5, 6.2, 7.8, 11.5, 10.5, 13.4, and 18.5 degrees.

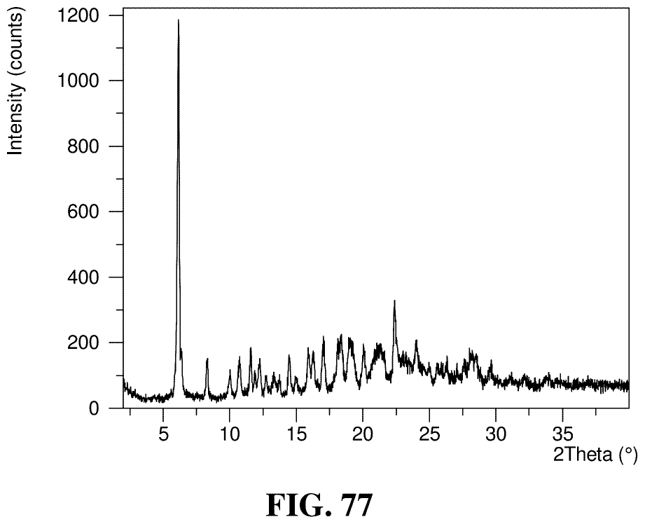

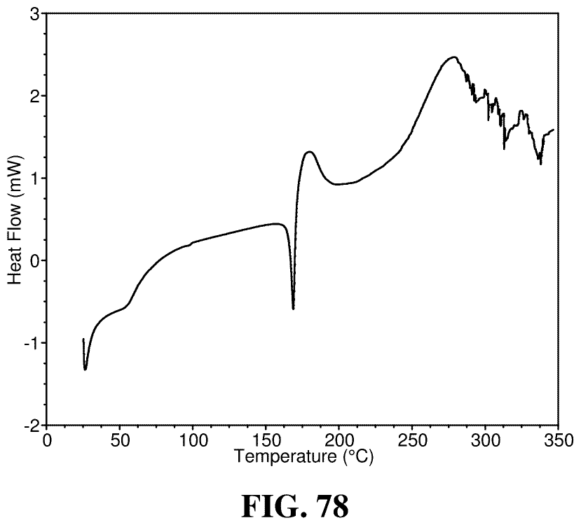

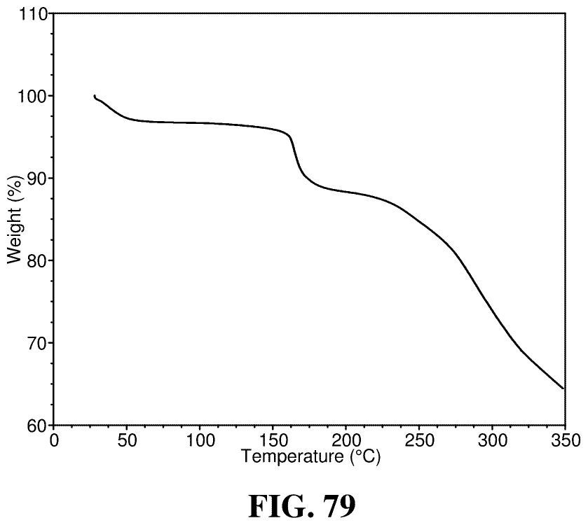

In some embodiments, provided herein is a Formula I R-Mandelate Form B, wherein the solid form exhibits an X-ray powder diffraction (XRPD) pattern substantially as shown in FIG. 77. Formula I R-Mandelate Form B may exhibit a differential scanning calorimetry (DSC) thermogram substantially as shown in FIG. 78. Formula I R-Mandelate Form B may exhibit a thermogravimetric analysis (TGA) thermogram substantially as shown in FIG. 79.

In some embodiments, Formula I R-Mandelate Form B has at least one, at least two, or all of the following properties:
(a) an XRPD pattern substantially as shown in FIG. 77
(b) a DSC thermogram substantially as shown in FIG. 78
(c) a TGA thermogram substantially as shown in FIG. 79.

In some embodiments, Formula I R-Mandelate Form B has an XRPD pattern displaying at least two, at least three, at least four, at least five, at least six, at least seven, at least eight, or at least nine of the degree 2θ-reflections with the greatest intensity as the XRPD pattern substantially as shown in FIG. 77.

In certain embodiments, Formula I R-Mandelate Form B has an XRPD pattern comprising degree 2θ-reflections (±0.2 degrees 2θ) at 6.2, 8.3, and 22.4 degrees. In some embodiments, Formula I R-Mandelate Form B has an XRPD pattern comprising degree 2θ-reflections (±0.2 degrees 2θ) at 6.2, 8.3, and 22.4 degrees and one, two, or three of the degree 2θ-reflections (±0.2 degrees 2θ) at 15.9, 16.3, and 17.0 degrees. In some embodiments, Formula I R-Mandelate Form B has an XRPD pattern comprising degree 2θ-reflections (±0.2 degrees 2θ) at 6.2, 8.3, and 22.4 degrees and one, two, or three of the degree 2θ-reflections (±0.2 degrees 2θ) at 10.0, 10.8, and 11.6 degrees. In some embodiments, Formula I R-Mandelate Form B has an XRPD pattern comprising degree 2θ-reflections (±0.2 degrees 2θ) at 6.2, 8.3, 22.4, 15.9, 16.3, 17.0, 10.0, 10.8, and 11.6 degrees.

### Formula I Benzoate

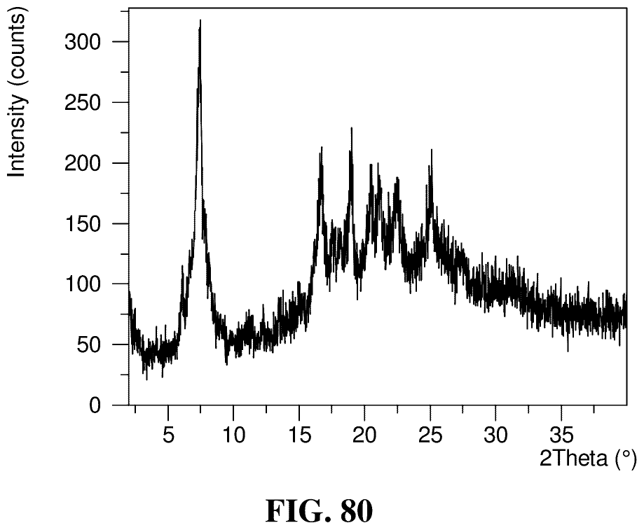

In some embodiments, provided herein is a Formula I Benzoate Form A, wherein the solid form exhibits an X-ray powder diffraction (XRPD) pattern substantially as shown in FIG. 80.

In some embodiments, Formula I Benzoate Form A has an XRPD pattern displaying at least two, at least three, at least four, at least five, at least six, or at least seven of the degree 2θ-reflections with the greatest intensity as the XRPD pattern substantially as shown in FIG. 80.

In certain embodiments, Formula I Benzoate Form A has an XRPD pattern comprising degree 2θ-reflections (±0.2 degrees 2θ) at 7.44, 18.97, and 16.70 degrees. In some embodiments, Formula I Benzoate Form A has an XRPD pattern comprising degree 2θ-reflections (±0.2 degrees 2θ) at 7.44, 18.97, and 16.70 degrees and one, two, or three of the degree 2θ-reflections (±0.2 degrees 2θ) at 21.14, 22.45, 25.07, and 6.09 degrees. In some embodiments, Formula I Benzoate Form A has an XRPD pattern comprising degree 2θ-reflections (±0.2 degrees 2θ) at 7.44, 18.97, 16.70, 21.14, 22.45, 25.07, and 6.09 degrees.

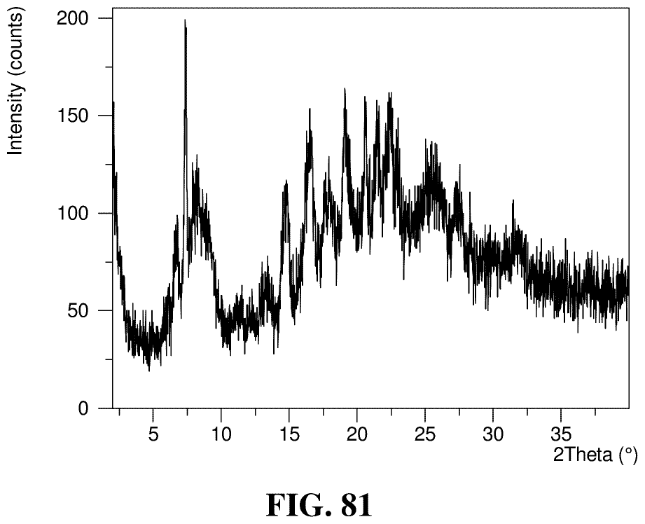

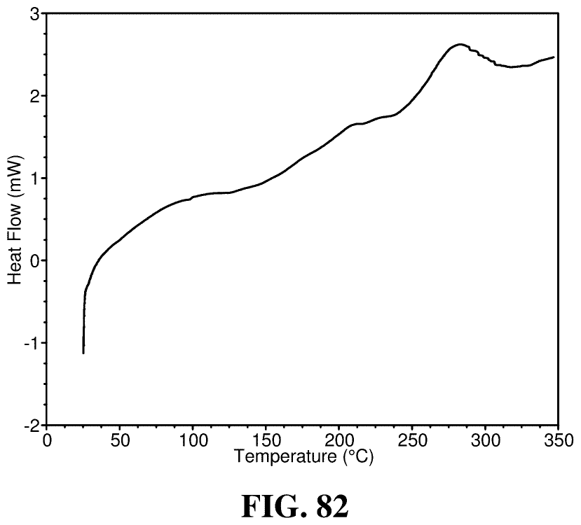

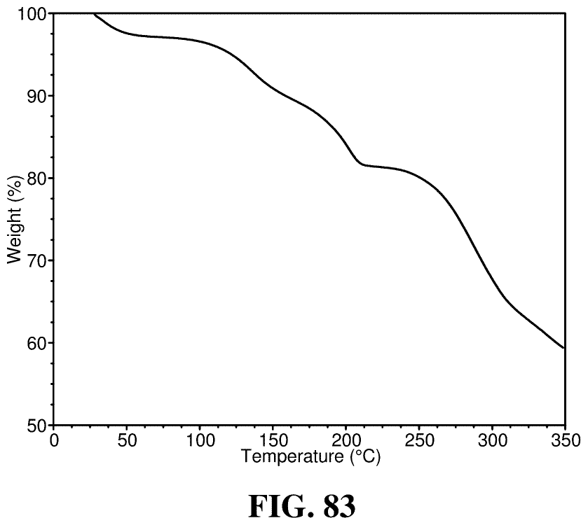

In some embodiments, provided herein is a Formula I Benzoate Form B, wherein the solid form exhibits an X-ray powder diffraction (XRPD) pattern substantially as shown in FIG. 81. Formula I Benzoate Form B may exhibit a differential scanning calorimetry (DSC) thermogram substantially as shown in FIG. 82. Formula I Benzoate Form B may exhibit a thermogravimetric analysis (TGA) thermogram substantially as shown in FIG. 83.

In some embodiments, Formula I Benzoate Form B has at least one, at least two, or at least three of the following properties:
(a) an XRPD pattern substantially as shown in FIG. 81
(b) a DSC thermogram substantially as shown in FIG. 82
(c) a TGA thermogram substantially as shown in FIG. 83.

In some embodiments, Formula I Benzoate Form B has an XRPD pattern displaying at least two, at least three, at least four, at least five, at least six, at least seven, at least eight, or at least nine of the degree 2θ-reflections with the greatest intensity as the XRPD pattern substantially as shown in FIG. 81.

In certain embodiments, Formula I Benzoate Form B has an XRPD pattern comprising degree 2θ-reflections (±0.2 degrees 2θ) at 14.9, 6.7, and 7.4 degrees. In some embodiments, Formula I Benzoate Form B has an XRPD pattern comprising degree 2θ-reflections (±0.2 degrees 2θ) at 14.9, 6.7, and 7.4 degrees and one, two, or three of the degree 2θ-reflections (±0.2 degrees 2θ) at 20.6, 22.5, and 8.1 degrees. In some embodiments, Formula I Benzoate Form B has an XRPD pattern comprising degree 2θ-reflections (±0.2 degrees 2θ) at 14.9, 6.7, and 7.4 degrees and one, two, or three of the degree 2θ-reflections (±0.2 degrees 2θ) at 6.7, 14.9, and 21.5 degrees. In some embodiments, Formula I Benzoate Form B has an XRPD pattern comprising degree 2θ-reflections (±0.2 degrees 2θ) at 14.9, 6.7, 7.4, 20.6, 22.5, 8.1, 6.7, 14.9, and 21.5 degrees.

### Formula I Methylparabenate

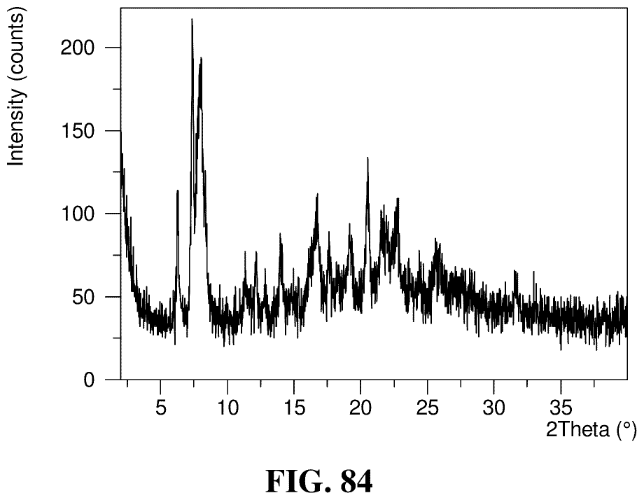

In some embodiments, provided herein is a Formula I Methylparabenate Form A, wherein the solid form exhibits an X-ray powder diffraction (XRPD) pattern substantially as shown in FIG. 84.

In some embodiments, Formula I Methylparabenate Form A has an XRPD pattern displaying at least two, at least three, at least four, at least five, at least six, at least seven, at least eight, or at least nine of the degree 2θ-reflections with the greatest intensity as the XRPD pattern substantially as shown in FIG. 84.

In certain embodiments, Formula I Methylparabenate Form A has an XRPD pattern comprising degree 2θ-reflections (±0.2 degrees 2θ) at 7.4, 8.0, and 6.3 degrees. In some embodiments, Formula I Methylparabenate Form A has an XRPD pattern comprising degree 2θ-reflections (±0.2 degrees 2θ) at 7.4, 8.0, and 6.3 degrees and one, two, or three of the degree 2θ-reflections (±0.2 degrees 2θ) at 20.5, 19.2, and 12.1 degrees. In some embodiments, Formula I Methylparabenate Form A has an XRPD pattern comprising degree 2θ-reflections (±0.2 degrees 2θ) at 7.4, 8.0, and 6.3 degrees and one, two, or three of the degree 2θ-reflections (±0.2 degrees 2θ) at 31.6, 22.8, and 14.0 degrees. In some embodiments, Formula I Methylparabenate Form A has an XRPD pattern comprising degree 2θ-reflections (±0.2 degrees 2θ) at 7.4, 8.0, 6.3, 20.5, 19.2, 12.1, 31.6, 22.8, and 14.0 degrees.

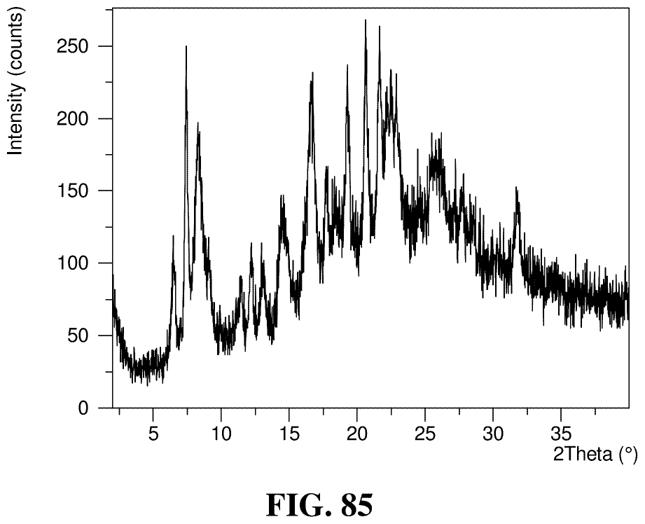

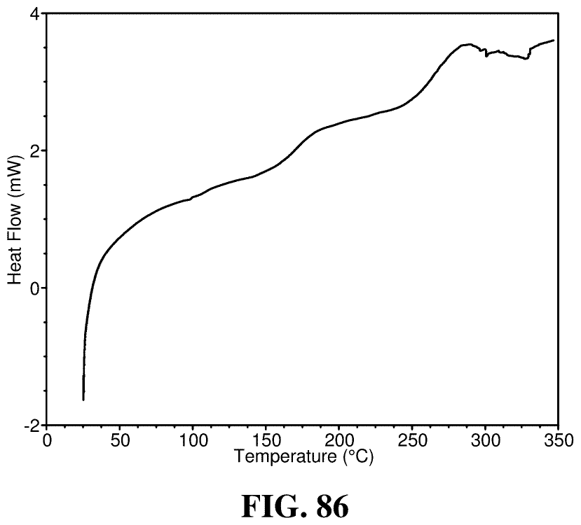

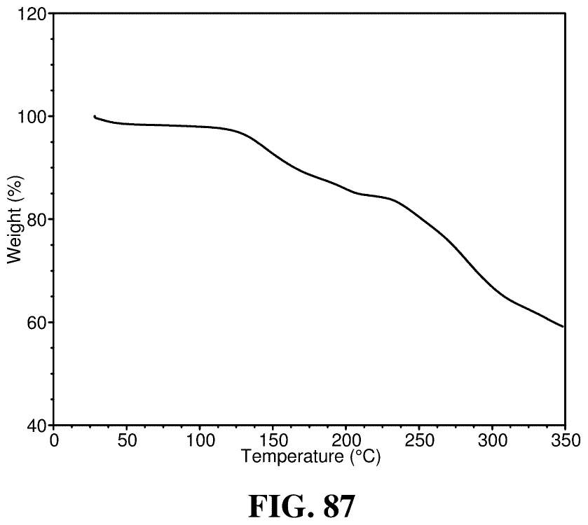

In some embodiments, provided herein is a Formula I Methylparabenate Form B, wherein the solid form exhibits an X-ray powder diffraction (XRPD) pattern substantially as shown in FIG. 85. Formula I Methylparabenate Form B may exhibit a differential scanning calorimetry (DSC) thermogram substantially as shown in FIG. 86. Formula I Methylparabenate Form B may exhibit a thermogravimetric analysis (TGA) thermogram substantially as shown in FIG. 87.

In some embodiments, Formula I Methylparabenate Form B has at least one, at least two, or at least three of the following properties:
(a) an XRPD pattern substantially as shown in FIG. 85
(b) a DSC thermogram substantially as shown in FIG. 86
(c) a TGA thermogram substantially as shown in FIG. 87.

In some embodiments, Formula I Methylparabenate Form B has an XRPD pattern displaying at least two, at least three, at least four, at least five, at least six, at least seven, at least eight, or at least nine of the degree 2θ-reflections with the greatest intensity as the XRPD pattern substantially as shown in FIG. 85.

In certain embodiments, Formula I Methylparabenate Form B has an XRPD pattern comprising degree 2θ-reflections (±0.2 degrees 2θ) at 7.4, 8.3, and 6.5 degrees. In some embodiments, Formula I Methylparabenate Form B has an XRPD pattern comprising degree 20-reflections (±0.2 degrees 2θ) at 7.4, 8.3, and 6.5 degrees and one, two, or three of the degree 2θ-reflections (±0.2 degrees 2θ) at 19.3, 20.6, and 21.6 degrees. In some embodiments, Formula I Methylparabenate Form B has an XRPD pattern comprising degree 2θ-reflections (±0.2 degrees 2θ) at 7.4, 8.3, and 6.5 degrees and one, two, or three of the degree 2θ-reflections (±0.2 degrees 2θ) at 12.2, 13.0, and 31.8 degrees. In some embodiments, Formula I Methylparabenate Form B has an XRPD pattern comprising degree 2θ-reflections (±0.2 degrees 2θ) at 7.4, 8.3, 6.5, 19.3, 20.6, 21.6, 12.2, 13.0, and 31.8 degrees.

### Formula I Caffeate

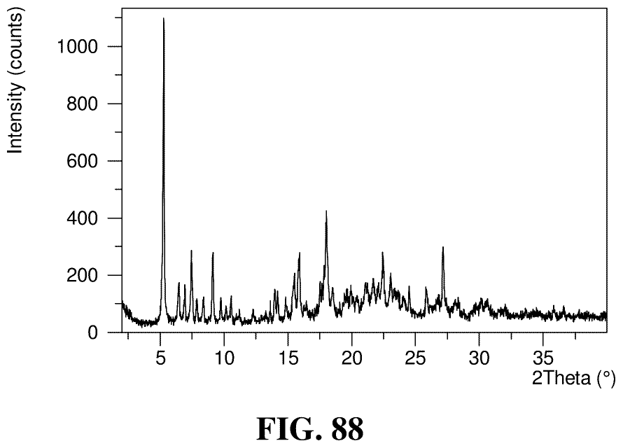

In some embodiments, provided herein is a Formula I Caffeate wet cake, wherein the solid form exhibits an X-ray powder diffraction (XRPD) pattern substantially as shown in FIG. 88.

In some embodiments, Formula I Caffeate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 88.

In certain embodiments, Formula I Caffeate wet cake has an XRPD pattern comprising degree 2θ-reflections (±0.2 degrees 2θ) at 5.3, 7.4, and 9.1 degrees. In some embodiments, Formula I Caffeate wet cake has an XRPD pattern comprising degree 2θ-reflections (±0.2 degrees 2θ) at 5.3, 7.4, and 9.1 degrees and one, two, or three of the degree 2θ-reflections (±0.2 degrees 2θ) at 6.5, 7.0, and 15.9 degrees. In some embodiments, Formula I Caffeate wet cake has an XRPD pattern comprising degree 2θ-reflections (±0.2 degrees 2θ) at 5.3, 7.4, and 9.1 degrees and one, two, or three of the degree 2θ-reflections (±0.2 degrees 2θ) at 27.1, 22.5, and 10.6 degrees. In some embodiments, Formula I Caffeate wet cake has an XRPD pattern comprising degree 2θ-reflections (±0.2 degrees 2θ) at 5.3, 7.4, 9.1, 6.5, 7.0, 15.9, 27.1, 22.5, and 10.6 degrees.

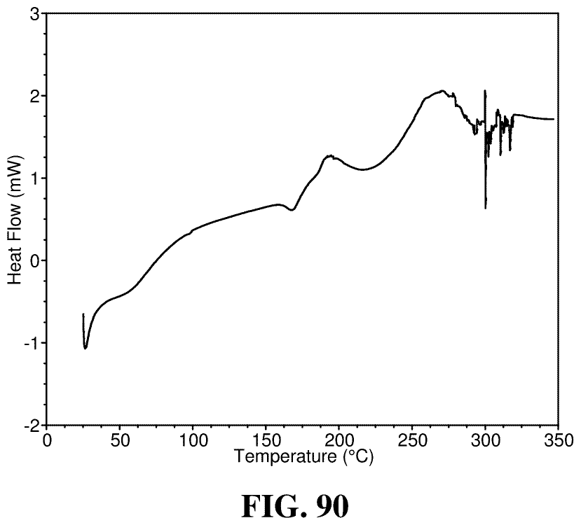

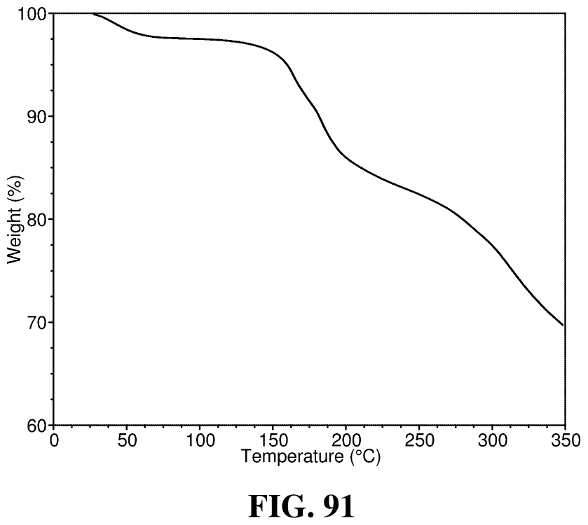

In some embodiments, provided herein is a solid form of Formula I Caffeate, wherein the solid form exhibits an X-ray powder diffraction (XRPD) pattern substantially as shown in FIG. 89. Formula I Caffeate may exhibit a differential scanning calorimetry (DSC) thermogram substantially as shown in FIG. 90. Formula I Caffeate may exhibit a thermogravimetric analysis (TGA) thermogram substantially as shown in FIG. 91.

In some embodiments, a solid form of Formula I Caffeate has at least one, at least two, or at least three of the following properties:
(a) an XRPD pattern substantially as shown in FIG. 89
(b) a DSC thermogram substantially as shown in FIG. 90
(c) a TGA thermogram substantially as shown in FIG. 91.

### Formula I Glycolate

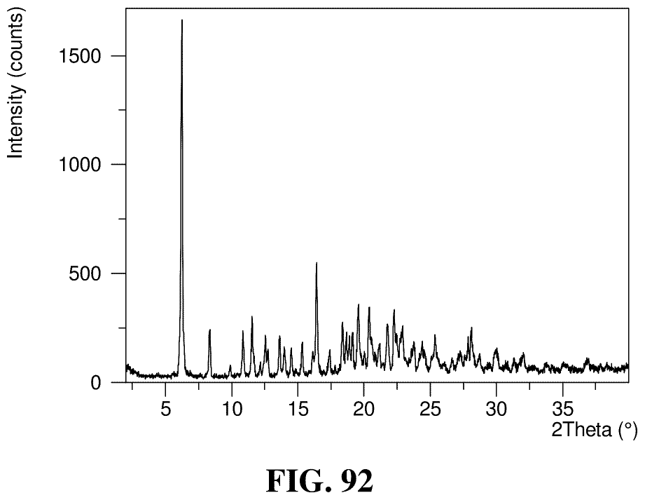

In some embodiments, provided herein is a Formula I Glycolate wet cake, wherein the solid form exhibits an X-ray powder diffraction (XRPD) pattern substantially as shown in FIG. 92.

In some embodiments, Formula I Glycolate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 92.

In certain embodiments, Formula I Glycolate wet cake has an XRPD pattern comprising degree 2θ-reflections (±0.2 degrees 2θ) at 6.2, 8.4, and 11.5 degrees. In some embodiments, Formula I Glycolate wet cake has an XRPD pattern comprising degree 2θ-reflections (±0.2 degrees 2θ) at 6.2, 8.4, and 11.5 degrees and one, two, or three of the degree 2θ-reflections (±0.2 degrees 2θ) at 10.9, 16.4, and 22.3 degrees. In some embodiments, Formula I Glycolate wet cake has an XRPD pattern comprising degree 2θ-reflections (±0.2 degrees 2θ) at 6.2, 8.4, and 11.5 degrees and one, two, or three of the degree 2θ-reflections (±0.2 degrees 2θ) at 13.6, 14.0, and 15.3 degrees. In some embodiments, Formula I Glycolate wet cake has an XRPD pattern comprising degree 2θ-reflections (±0.2 degrees 2θ) at 6.2, 8.4, 11.5, 10.9, 16.4, 22.3, 13.6, 14.0, and 15.3 degrees.

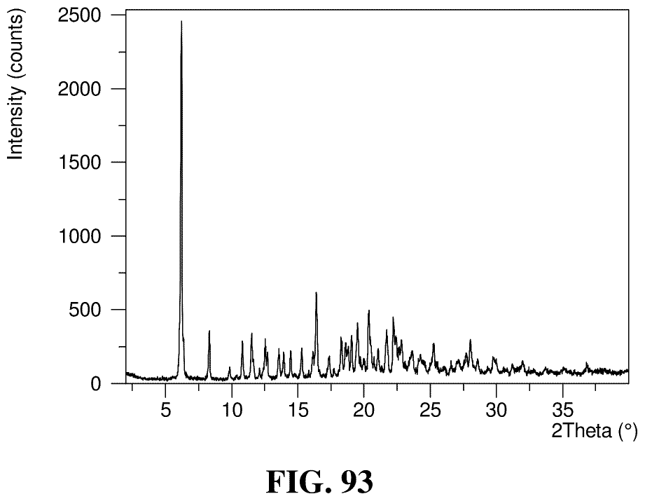

In some embodiments, provided herein is a solid form of Formula I Glycolate, wherein the solid form exhibits an X-ray powder diffraction (XRPD) pattern substantially as shown in FIG. 93.

In some embodiments, Formula I Glycolate has an XRPD pattern displaying at least two, at least three, at least four, at least five, at least six, at least seven, at least eight, or at least nine of the degree 2θ-reflections with the greatest intensity as the XRPD pattern substantially as shown in FIG. 93.

In certain embodiments, Formula I Glycolate has an XRPD pattern comprising degree 2θ-reflections (±0.2 degrees 2θ) at 6.2, 8.3, and 11.5 degrees. In some embodiments, Formula I Glycolate has an XRPD pattern comprising degree 2θ-reflections (±0.2 degrees 2θ) at 6.2, 8.3, and 11.5 degrees and one, two, or three of the degree 2θ-reflections (±0.2 degrees 2θ) at 10.8, 16.4, and 22.2 degrees. In some embodiments, Formula I Glycolate has an XRPD pattern comprising degree 2θ-reflections (±0.2 degrees 2θ) at 6.2, 8.3, and 11.5 degrees and one, two, or three of the degree 2θ-reflections (±0.2 degrees 2θ) at 13.6, 14.9, and 15.3 degrees. In some embodiments, Formula I Glycolate has an XRPD pattern comprising degree 2θ-reflections (±0.2 degrees 2θ) at 6.2, 8.3, 11.5, 10.8, 16.4, 22.2, 13.6, 14.9, and 15.3 degrees.

### Formula I α-Ketobutyrate

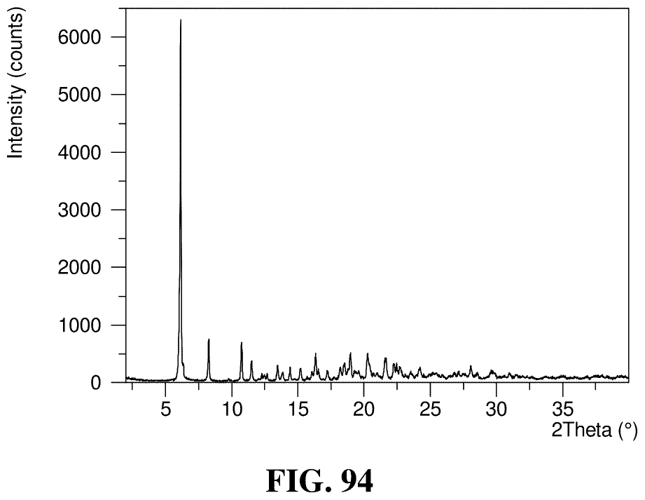

In some embodiments, provided herein is a Formula I α-Ketobutyrate wet cake, wherein the Formula I α-Ketobutyrate wet cake exhibits an X-ray powder diffraction (XRPD) pattern substantially as shown in FIG. 94.

In some embodiments, Formula I α-Ketobutyrate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 94.

In certain embodiments, Formula I α-Ketobutyrate wet cake has an XRPD pattern comprising degree 2θ-reflections (±0.2 degrees 2θ) at 6.2, 8.3, and 10.8 degrees. In some embodiments, Formula I α-Ketobutyrate wet cake has an XRPD pattern comprising degree 2θ-reflections (±0.2 degrees 2θ) at 6.2, 8.3, and 10.8 degrees and one, two, or three of the degree 2θ-reflections (±0.2 degrees 2θ) at 12.3, 12.5, and 12.7 degrees. In some embodiments, Formula I α-Ketobutyrate wet cake has an XRPD pattern comprising degree 2θ-reflections (±0.2 degrees 2θ) at 6.2, 8.3, and 10.8 degrees and one, two, or three of the degree 2θ-reflections (±0.2 degrees 2θ) at 16.4, 19.0, and 21.6 degrees. In some embodiments, Formula I α-Ketobutyrate wet cake has an XRPD pattern comprising degree 2θ-reflections (±0.2 degrees 2θ) at 6.2, 8.3, 10.8, 12.3, 12.5, 12.7, 16.4, 19.0, and 21.6 degrees.

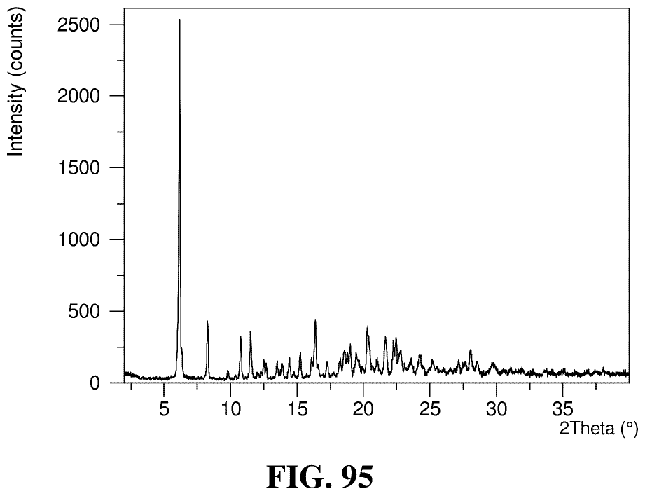

In some embodiments, provided herein is a solid form of Formula I α-Ketobutyrate, wherein the solid form exhibits an X-ray powder diffraction (XRPD) pattern substantially as shown in FIG. 95.

In some embodiments, Formula I α-Ketobutyrate has an XRPD pattern displaying at least two, at least three, at least four, at least five, at least six, at least seven, at least eight, or at least nine of the degree 2θ-reflections with the greatest intensity as the XRPD pattern substantially as shown in FIG. 95.

In certain embodiments, Formula I α-Ketobutyrate has an XRPD pattern comprising degree 2θ-reflections (±0.2 degrees 2θ) at 6.2, 16.4, and 8.3 degrees. In some embodiments, Formula I α-Ketobutyrate has an XRPD pattern comprising degree 2θ-reflections (±0.2 degrees 2θ) at 6.2, 16.4, and 8.3 degrees and one, two, or three of the degree 2θ-reflections (±0.2 degrees 2θ) at 10.8, 11.5, and 20.3 degrees. In some embodiments, Formula I α-Ketobutyrate has an XRPD pattern comprising degree 2θ-reflections (±0.2 degrees 2θ) at 6.2, 16.4, and 8.3 degrees and one, two, or three of the degree 2θ-reflections (±0.2 degrees 2θ) at 18.5, 19.0, and 21.6 degrees. In some embodiments, Formula I α-Ketobutyrate has an XRPD pattern comprising degree 2θ-reflections (±0.2 degrees 2θ) at 6.2, 16.4, 8.3, 10.8, 11.5, 20.3, 18.5, 19.0, and 21.6 degrees.

### Formula I Pyruvate

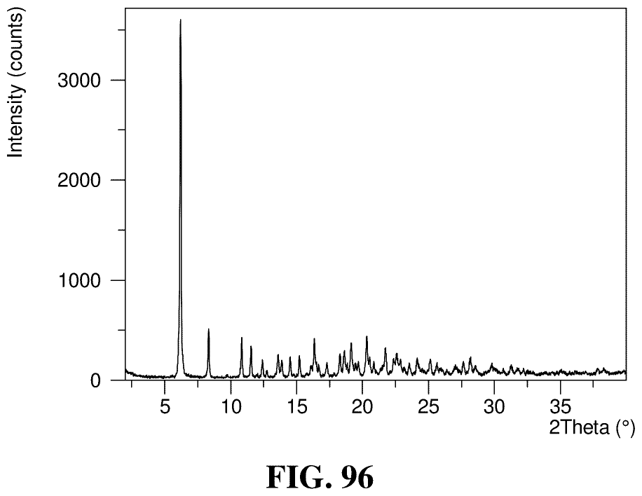

In some embodiments, provided herein is a Formula I Pyruvate wet cake, wherein the Formula I Pyruvate wet cake exhibits an X-ray powder diffraction (XRPD) pattern substantially as shown in FIG. 96.

In some embodiments, Formula I Pyruvate wet cake has an XRPD pattern displaying at least two, at least three, at least four, at least five, at least six, at least seven, at least eight, or at least nine of the degree 2θ-reflections with the greatest intensity as the XRPD pattern substantially as shown in FIG. 96.

In certain embodiments, Formula I Pyruvate wet cake has an XRPD pattern comprising degree 2θ-reflections (±0.2 degrees 2θ) at 6.21, 8.34, and 10.85 degrees. In some embodiments, Formula I Pyruvate wet cake has an XRPD pattern comprising degree 2θ-reflections (±0.2 degrees 2θ) at 6.21, 8.34, and 10.85 degrees and one, two, or three of the degree 2θ-reflections (±0.2 degrees 2θ) at 11.56, 13.60, and 14.51 degrees. In some embodiments, Formula I Pyruvate wet cake has an XRPD pattern comprising degree 2θ-reflections (±0.2 degrees 2θ) at 6.21, 8.34, and 10.85 degrees and one, two, or three of the degree 2θ-reflections (±0.2 degrees 2θ) at 20.34, 21.73, and 22.59 degrees. In some embodiments, Formula I Pyruvate wet cake has an XRPD pattern comprising degree 2θ-reflections (±0.2 degrees 2θ) at 6.21, 8.34, 10.85, 11.56, 13.60, 14.51, 20.34, 21.73, and 22.59 degrees.

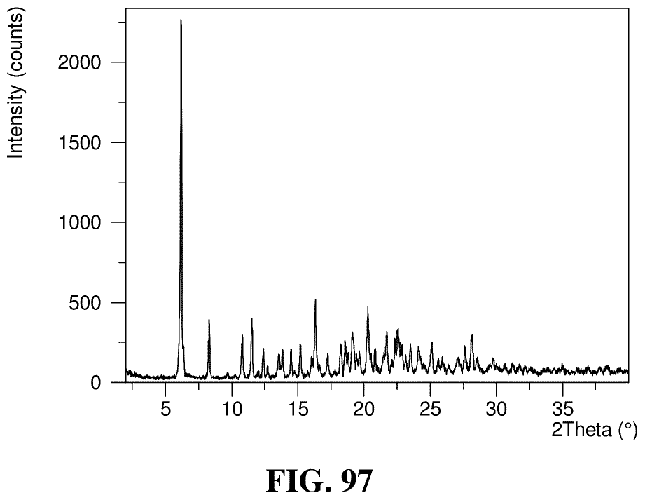

In some embodiments, provided herein is a solid form of Formula I Pyruvate, wherein the solid form exhibits an X-ray powder diffraction (XRPD) pattern substantially as shown in FIG. 97.

In some embodiments, Formula I Pyruvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 97.

In certain embodiments, Formula I Pyruvate has an XRPD pattern comprising degree 2θ-reflections (±0.2 degrees 2θ) at 6.2, 8.3, and 10.8 degrees. In some embodiments, Formula I Pyruvate has an XRPD pattern comprising degree 2θ-reflections (±0.2 degrees 2θ) at 6.2, 8.3, and 10.8 degrees and one, two, or three of the degree 2θ-reflections (±0.2 degrees 2θ) at 11.5, 16.3, and 19.1 degrees. In some embodiments, Formula I Pyruvate has an XRPD pattern comprising degree 2θ-reflections (±0.2 degrees 2θ) at 6.2, 8.3, and 10.8 degrees and one, two, or three of the degree 2θ-reflections (±0.2 degrees 2θ) at 20.3, 21.7, and 22.1 degrees. In some embodiments, Formula I Pyruvate has an XRPD pattern comprising degree 2θ-reflections (±0.2 degrees 2θ) at 6.2, 8.3, 10.8, 11.5, 16.3, 19.1, 20.3, 21.7, and 22.1 degrees.

### Pharmaceutical Compositions

For the purposes of administration, in certain embodiments, the compounds described herein are administered as a raw chemical or are formulated as pharmaceutical compositions. Pharmaceutical compositions of the present disclosure can include a therapeutically effective amount of a compound of Formula I, and at least one pharmaceutically acceptable carrier and/or excipient. The compound of Formula I is present in the composition in an amount which is effective to treat a particular disease or condition of interest. The pharmaceutical compositions of the present disclosure may additionally comprise one or more other compounds as active ingredients, including for instance prodrugs, other nuclear receptor modulators, or other active pharmaceutical ingredients such as active pharmaceutical ingredients for use in treating liver disease, such as ACC inhibitors or ASK1 inhibitors. In some embodiments, the pharmaceutical compositions of the present disclosure additionally comprise an ACC inhibitor and an ASK1 inhibitor.

In some embodiments, a pharmaceutical composition includes amorphous Formula I; Formula I Form I; Formula I Form II; Formula I Form III; Formula I Form IV; Formula I Form V; Formula I Form VI; Formula I Form VII; Formula I Form VIII; Formula I Form IX; Formula I Form X; Formula I Form XI; Formula I Form XII; Formula I Form XIII; Formula I Form XIV; Formula I Form XV; Formula I 2-(4-Hydroxybenzoyl) benzoate Form A; Formula I 2-(4-Hydroxybenzoyl) benzoate Form B Formula I Vanillate Form A; Formula I Vanillate Form B; Formula I Hippurate Form A; Formula I Hippurate Form B; Formula I Maleate Form A; Formula I Maleate Form B; Formula I glyoxylate Form A; Formula I glyoxylate Form B; Formula I L-Pyroglutamate; Formula I 2-Naphthalene sulfonate Form A; Formula I 2-Naphthalene sulfonate Form B; Formula I 1-Naphthalene sulfonate Form A; Formula I 1-Naphthalene sulfonate Form B; Formula I 1-Hydroxy-2-Naphthoate Form A; Formula I 1-Hydroxy-2-Naphthoate Form B; Formula I S-Mandelate Form A; Formula I S-Mandelate Form B; Formula I Gentisate Form A; Formula I Gentisate Form B; Formula I Citrate Form A; Formula I Citrate Form B; Formula I R-Mandelate Form A; Formula I R-Mandelate Form B; Formula I Benzoate Form A; Formula I Benzoate Form B; Formula I Methylparabenate Form A; Formula I Methylparabenate Form B; Formula I Caffeate Form A; Formula I Caffeate Form B; Formula I Glycolate Form A; Formula I Glycolate Form B; Formula I α-Ketobutyrate Form A; Formula I α-Ketobutyrate Form B; Formula I Pyruvate Form A; and/or Formula I Pyruvate Form B and a pharmaceutically acceptable carrier.

The activity of compounds of Formula I can be determined by one skilled in the art, for example, as described herein. Appropriate therapeutically effective concentrations and dosages can be readily determined by one skilled in the art.

In certain embodiments, the crystalline, salt, and/or solvate forms described herein may potentially exhibit improved properties. For example, in certain embodiments, the crystalline and/or salt forms described herein may potentially exhibit improved stability. Such improved stability could have a potentially beneficial impact on the manufacture of the compound of Formula I, such as for example offering the ability to store process intermediate for extended periods of time. Improved stability could also potentially benefit a composition or pharmaceutical composition of the compound of Formula I. In certain embodiments, the crystalline salt, and/or solvate forms described herein may also potentially result in improved yield of the compound of Formula I, or in an improvement of the quality of the compound of Formula I. In certain embodiments, the crystalline, salt, and/or solvate forms described herein may also exhibit improved pharmacokinetic properties and/or potentially improved bioavailability.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of the present disclosure can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate, microcrystalline cellulose, lactose monohydrate, mannitol or colloidal silicon dioxide; a disintegrating agent such as corn starch, potato starch, alginic acid, croscarmellose sodium or crospovidone; a lubricant such as magnesium stearate; and a sweetening agent such as sucrose, lactose or saccharin. When a dosage unit form is a capsule, it may contain, in addition to materials of the above type, a liquid carrier such as a fatty oil or pharmaceutical media, such as, for example, water, glycols (e.g., polyethylene glycol 400), or alcohols.

Various other materials may be present as coatings or to modify the physical form of the dosage unit. For instance, tablets may be coated with shellac, sugar, polyvinyl alcohol, polyethylene glycol 3350, titanium dioxide, talc, coloring agent, or combinations thereof. A syrup or elixir may contain, in addition to the active ingredient, sucrose as a sweetening agent, methyl and propylparabens as preservatives, a dye and a flavoring such as cherry or orange flavor.

The compounds of the present disclosure may also be administered parenterally. Solutions or suspensions of these active compounds can be prepared in water suitably mixed with an organic, an additive, or combinations thereof. Examples of organics include, but are not limited to, N-methyl pyrrolidone, dimethylsulfoxide, polyethylene glycols, and combinations thereof. Examples of additives include, but are not limited to, hydroxypropyl cellulose, polyvinylpyrrolidone, poloxamers, poly(lactic-co-glycolic acid), polysorbates, povidone, carboxymethylcellulose, and combinations thereof. Dispersions can also be prepared in glycerol, liquid polyethylene glycols and mixtures thereof in oils. Under ordinary conditions of storage and use, these preparations may contain a preservative to, for instance, prevent the growth of microorganisms. In some embodiments, the parenteral administration includes intravenous administration with formulations comprising solutions with a mixture of organics and aqueous media. In some embodiments, the intravenous administration is dosed as a 100% organic solution.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. For instance, the forms can be stable under the conditions of manufacture and storage. The forms can be preserved against the contaminating action of microorganisms such as bacteria and fungi (for instance, via use of preservatives). The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present disclosure. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In some embodiments, compounds of the present disclosure are administered orally.

In particular embodiments of compositions comprising a crystalline form of Formula I or a pharmaceutically acceptable salt thereof, at least about 50%, at least about 60%, at least about 70%, at least about 80%, at least about 85%, at least about 90%, at least about 95%, at least about 96%, at least about 97%, at least about 98%, or at least about 99% of Formula I present in the composition is one of the crystalline forms disclosed herein. In certain embodiments, the composition includes at least about 50%, at least about 60%, at least about 70%, at least about 80%, at least about 85%, at least about 90%, at least about 95%, at least about 96%, at least about 97%, at least about 98%, or at least about 99% of one of the crystalline forms of Formula I.

In other embodiments of compositions comprising a crystalline form disclosed herein, less than about 50%, less than about 40%, less than about 30%, less than about 20%, less than about 10%, less than about 5%, less than about 4%, less than about 3%, less than about 2% or less than about 1% of Formula I present in the composition are other amorphous or crystal forms of Formula I and/or impurities.

In yet other embodiments of compositions comprising the crystalline forms disclosed herein, impurities make up less than about 5%, less than about 4%, less than about 3%, less than about 2% or less than about 1% of the total mass relative to the mass of the crystalline forms present. Impurities may, for example, include by-products from synthesizing Formula I, contaminants, degradation products, other crystalline forms, amorphous form, water, and solvents. In certain embodiments, impurities include by-products from the process of synthesizing Formula I. In certain embodiments, impurities include contaminants from the process of synthesizing Formula I. In certain embodiments, impurities include degradation products of Formula I. In certain embodiments, impurities include other crystalline forms of Formula I. In certain embodiments, impurities include other crystalline forms of Formula I and/or amorphous forms of Formula I. In certain embodiments, impurities include water or solvent. In certain embodiments of compositions comprising a crystalline form disclosed herein, impurities are selected from the group consisting of by-products from synthesizing Formula I, contaminants, degradation products, other crystalline forms, amorphous forms, water, solvents and combinations thereof.

### Dosage

The effective dosage of active ingredient employed may vary depending on the particular compound employed, the mode of administration, the condition being treated and the severity of the condition being treated. Such dosage may be ascertained readily by a person skilled in the art.

When treating or preventing Cot mediated conditions for which compounds of the present disclosure are indicated, generally satisfactory results are obtained when the compounds of the present disclosure are administered at a daily dosage of from about 0.1 milligram to about 100 milligram per kilogram of animal body weight. In some embodiments, the compounds of the present disclosure are given as a single daily dose or in divided doses two to six times a day, or in sustained release form. For most large mammals, the total daily dosage can be from about 1 milligram to about 1000 milligrams. In the case of a 70 kg adult human, the total daily dose will generally be from about 7 milligrams to about 350 milligrams. This dosage regimen may be adjusted to provide the optimal therapeutic response. In some embodiments, the total daily dosage is from about 1 milligram to about 900 milligrams, about 10 milligrams to about 800 milligrams, about 20 milligrams to about 700 milligrams, about 30 milligrams to about 600 milligrams, about 40 milligrams to about 550 milligrams, or about 50 milligrams to about 400 milligrams. In some embodiments, the total daily dosage is from about 10 milligrams to about 50 milligrams, from about 20 milligrams to about 40 milligrams, from about 25 milligrams to about 35 milligrams, from about 50 milligrams to about 150 milligrams, from about 70 milligrams to about 130 milligrams, from about 80 milligrams to about 120 milligrams, from about 90 milligrams to about 100 milligrams, from about 1 milligram to about 150 milligrams, from about 1 milligram to about 75 milligrams, from about 1 milligram to about 50 milligrams, from about 25 milligrams to about 125 milligrams, from about 125 milligrams to about 275 milligrams, from about 275 milligrams to about 425 milligrams, from about 425 milligrams to about 575 milligrams, from about 575 milligrams to about 725 milligrams, from about 725 milligrams to about 875 milligrams, or from about 875 milligrams to about 1000 milligrams.

The compounds of the present application or the compositions thereof may be administered once, twice, three, or four times daily, using any suitable mode described above. Also, administration or treatment with the compounds may be continued for a number of days or months; for example, commonly treatment would continue for at least 7 days, 14 days, or 28 days, or 1, 2, 3, 4, 5, 6, 7, 8, 9, 10, 11, or 12 months.

In a particular embodiment, the methods provided herein comprise administering to the subject an initial daily dose of about 1 mg to about 1500 mg of a compound described herein, such as 150 to 600 mg, for example 150 mg, 300 mg, 600 mg. In further embodiments, the methods comprise increasing the dose by increments until clinical efficacy is achieved. Increments of about 5, 10, 25, 30, 40, 50, or 100 mg can be used to increase the dose. The dosage can be increased daily, every other day, twice per week, once per week or once every 4 weeks.

### Treatment Methods and Uses

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The disclosure further relates to the use of said compounds for the treatment and/or prophylaxis of diseases and/or conditions through binding of said nuclear receptor by said compounds. Further the present disclosure relates to the use of said compounds for the preparation of a medicament for the treatment and/or prophylaxis of diseases and/or conditions through binding of said nuclear receptor by said compounds.

Also provided herein are methods of treating a patient having a Cot mediated condition. In some embodiments, the method includes administering a compound or composition disclosed herein. In some embodiments, a method of treating a patient having an Cot mediated condition includes administering a therapeutically effective amount of amorphous Formula I; Formula I Form I; Formula I Form II; Formula I Form III; Formula I Form IV; Formula I Form V; Formula I Form VI; Formula I Form VII; Formula I Form VIII; Formula I Form IX; Formula I Form X; Formula I Form XI; Formula I Form XII; Formula I Form XIII; Formula I Form XIV; Formula I Form XV; Formula I 2-(4-Hydroxybenzoyl) benzoate; Formula I Vanillate; Formula I Hippurate; Formula I Maleate; Formula I glyoxylate; Formula I L-Pyroglutamate; Formula I 2-Naphthalene sulfonate; Formula I 1-Naphthalene sulfonate; Formula I 1-Hydroxy-2-Naphthoate; Formula I S-Mandelate; Formula I Gentisate; Formula I Citrate; Formula I R-Mandelate; Formula I Benzoate; Formula I Methylparabenate; Formula I Caffeate; Formula I Glycolate; Formula I α-Ketobutyrate; and/or Formula I Pyruvate.

Also provided herein are methods of treating or preventing a disease or condition in a patient in need thereof, comprising administering a therapeutically effective amount of a compound useful for modulating Cot, wherein the disease or condition is an inflammatory disease, and wherein the compound useful for modulating Cot is a compound of Formula I

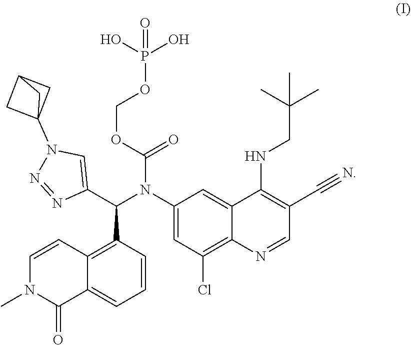 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

### Kits

Provided herein are also kits that include a compound or composition described herein and suitable packaging. In one embodiment, a kit further includes instructions for use. In one aspect, a kit includes a crystalline form of the disclosure, or composition including a crystalline form of the disclosure and a label and/or instructions for use of the compounds in the treatment of the indications, including the diseases or conditions, described herein.

Provided herein are also articles of manufacture that include a compound or composition described herein in a suitable container. The container may be a vial, jar, ampoule, preloaded syringe, and intravenous bag.

### Additional Therapeutic Agents

In some embodiments, a compound of the disclosure is co-administered with one or more (e.g., one, two, three, or four) additional therapeutic agents. In some embodiments, the additional therapeutic agent includes an agent useful for modulating, treating, or preventing inflammation, such as 5-HT 1a receptor partial agonists and antagonists, 5-HT 2a receptor partial agonists and antagonists, 5-HT 2b receptor antagonists, 5-HT 6 receptor antagonists, 5-HT 7 receptor antagonists, Abl tyrosine kinase inhibitors, ACE inhibitors, Acidic mammalian chitinase inhibitors, Actin antagonists, Acetaldehyde dehydrogenase inhibitors, Acetyl CoA carboxylase (ACC) inhibitors, ACC-1 inhibitors, ACC-2 inhibitors, 2-Acylglycerol O-acyltransferase 2 (DGAT2) inhibitors, ACTH receptor agonists, Activin receptor antagonists, Adenosylhomocysteinase inhibitors, Adenosine receptor antagonists and agonists, Adenosine deaminase inhibitors, Adenylyl cyclase associated protein 1 inhibitors, Adiponutrin inhibitors, Adiponectin receptor agonists, ADP ribosyl cyclase-1 inhibitors, ADP ribosyl cyclase-1 modulators, ADP ribosylation factor 6 inhibitors, Adrenocorticotrophic hormone ligands, adrenomedullin ligands, Adrenergic receptor antagonists and agonists, Adropin stimulators, Aggrecanase-2 inhibitors, AIMP multisynthetase complex protein 1 stimulators, AKT1 gene inhibitors, AKT protein kinase inhibitors, Albumin antagonists, Albumin modulators, Aldehyde dehydrogenase 2 stimulators, Aldosterone antagonists, Aldosterone synthase inhibitors Alk-5 protein kinase inhibitors, Alpha 2 adrenoceptor agonists, Alpha 2 adrenoceptor modulators, Alpha 1 antitrypsin stimulator, alpha-fetoprotein modulators, Alstrom syndrome protein 1 (ALMS1)/PKC alpha protein interaction inhibitors, 1 Aminocyclopropane carboxyl synthase inhibitors, Amylin receptor agonists, AMP-activated protein kinases (AMPK), AMP activated protein kinase inhibitors, activators or stimulators, AMP activated protein kinase alpha 2 stimulators, Androgen receptor agonists and antagonists, Angiopoietin-related protein-3 inhibitors, Angiotensin II receptor antagonists, Angiotensin II AT-1 receptor antagonists, Angiotensin II AT-2 receptor agonists, Angiotensinogen ligand inhibitors, Annexin A1 modulators, antibiotics, antifungals, anti-IL6 antibodies, anti-TNF steroid conjugates, activator protein 1 (APi) transcription factor inhibitors, APi transcription factor modulators, Apelin receptor agonists, APOA1 gene stimulators, Apolipoprotein A antagonists, Apolipoprotein B modulators, Apolipoprotein L1 modulators, Apoptosis regulator Bcl w inhibitors, Aryl hydrocarbon receptor (AHR) agonists and modulators, AHR agonist plus autoantigen, ASK1 inhibitors, ATPase inhibitors, ATP binding cassette transporter C2 inhibitors, ATP citrate lyase inhibitors, Autophagy protein modulators and stimulators, Autotaxin inhibitors, Axl tyrosine kinase receptor inhibitors, BAFF/APRIL inhibitors, Basigin inhibitors, B and T lymphocyte attenuator stimulators, Bax protein stimulators, Bcl-2 protein inhibitors, Bcl-xL Bcl-2 associated death promotor inhibitors, Bcl-xL Bcl-2 associated death promotor modulators, Bcr protein inhibitors, Benzodiazepine receptor agonists, beta adrenoceptor antagonists, BET inhibitors, Beta 2 adrenoceptor agonists, Beta amyloid antagonists, Beta-catenin inhibitors, Beta-catenin modulators, Beta-catenin stimulators, Beta-galactosidase inhibitors, beta lactamase modulators, 17 beta hydroxysteroid dehydrogenase 13 inhibitors, Bifunctional aminoacyl tRNA synthetase inhibitors, B-lymphocyte antigen CD19 inhibitors, B-lymphocyte antigen CD20 inhibitors, B-lymphocyte antigen CD20 modulators, B-lymphocyte cell adhesion molecule inhibitors, B-lymphocyte stimulator ligand inhibitors, B-lymphocyte stimulator ligand modulators, Bioactive lipids, Bone morphogenetic protein-7 ligand, Bone morphogenetic protein-7 ligand modulators, Bradykinin receptor modulators, BRAF gene inhibitors, Branched amino acid aminotransferase 1 inhibitors, Bromodomain containing protein (BRD) inhibitors, BRD1, BRD2, and BRD4 inhibitors, BTK inhibitors, B7 homolog inhibitor, Cadherin-11 antagonists, Cak tyrosine kinase receptor inhibitors, Calcineurin inhibitors, Calcium channel inhibitors, Ca2+ release activated Ca2+ channel 1 inhibitors, Calcitonin agonists, Calpain-IX inhibitors, Calpain-I inhibitors, Calpain-II inhibitors, Calreticulin inhibitors, Caveolin 1 stimulators, Cannabinoid CB1 receptor antagonists and inverse agonists, Cannabinoid CB2 receptor agonists, Cannabinoid receptor antagonists and agonists, Cannabinoid CB1 receptor inverse agonists, carbohydrate metabolism modulators, Carbonic anhydrase inhibitors, Casein kinase-I delta and/or epsiloninhibitors, CASP9 gene stimulators, Caspase inhibitors, Caspase-3 stimulators, Catalase stimulators, Cathepsin inhibitors, Cathepsin K inhibitors, Cathepsin S inhibitors, Caveolin 1 inhibitors, CCK receptor antagonists, CCAAT enhancer binding protein beta modulators, C-C motif ligand 26 (CCL26) gene inhibitors, Chemokine receptor antagonists, C-C motif chemokine receptor (CCR) 1 antagonists, CCR2 antagonists, CCR3 antagonists and modulators, CCR4 antagonists, CCR5 antagonists, CCR6 antagonists, CCR7 modulators, CCR9 chemokine antagonists, CCR3 gene modulators, CD3 modulators or antagonists, CD4 agonists or antagonists, CD7 inhibitors, CD11b agonists, CD29 modulators, CD39 agonists, CD40 ligand receptor modulators or antagonists, CD47 antagonists, CD52 antagonists, CD73 agonists and antagonists, CD79b modulators, CD80 modulators or antagonists, CD86 modulators or antagonists, CD95 antagonists, CD126 antagonists, CD223 modulators, CDGSH iron sulfur domain protein modulators, CDw123 antagonists, Cell adhesion molecule inhibitors, Cell surface glycoprotein CD200R agonists, Cell surface glycoprotein MUC18 inhibitors, chemokine CXC ligand inhibitors, Chaperonin inhibitors and modulators, chitinase inhibitors, Chitotriosidase 1 inhibitors, Chloride channel stimulators, Cholera enterotoxin subunit B inhibitors, Choline kinase inhibitors, CHST15 gene inhibitors, Chymase inhibitors, Claudin 1 inhibitors, Clusterin stimulators, CNR1 inhibitors, Collagen I antagonists, Collagen VII antagonists, Collagen gene inhibitors, Collagenase inhibitors, collagen modulators, Complement Clq subcomponent inhibitors, Complement CIs subcomponent inhibitors, Complement C3 inhibitors, Complement C5 factor inhibitors, Complement C5a receptor antagonists, Complement cascade inhibitors, Complement Factor stimulators, Complement Factor B inhibitors, Complement factor D inhibitors, Connective tissue growth factor ligand inhibitors, Corticosteroid hormone receptor agonists, COT protein kinase inhibitors, CREB binding protein inhibitors, C-reactive protein (CRP) inhibitors, cerebrospinal fluid (CSF)-1 agonists and antagonists, C-type lectin domain protein 4C inhibitors, CTGF gene inhibitors, CX3CR1 antagonists and modulators, CXCR2 antagonists, CXCR3 antagonist, CXCR4 antagonists and modulators, CXCR5 antagonists and modulators, CXC5 ligand inhibitors, CXC6 chemokine ligand inhibitors, CXC10 ligand inhibitors, CXC11 ligand modulators, Cyclin-dependent kinase (CDK) 1, 2, 5, 7, and/or 9 inhibitors, Cyclooxygenase (COX) inhibitors, COX-1 inhibitors, COX-2 inhibitors and modulators, Cysteine palmitoyltransferase porcupine inhibitors, Cytochrome P450 7A1 inhibitors, Cytochrome P450 11B2 inhibitors, Cytochrome P450 2E1 inhibitors (CYP2E1), Cytochrome P450 reductase inhibitors, Cytokine receptor agonists and antagonists, Cytosolic phospholipase A2 (cPLA2) inhibitors, Cytotoxic T-lymphocyte protein-4 (CTLA4) modulators and stimulators, Deoxyribonuclease (DNase) modulators, DNase gamma stimulators, DNase I stimulators, DGAT2 gene inhibitors, DHFR inhibitors, Diacylglycerol O acyltransferase (DGAT) 1 inhibitors, DGAT2 inhibitors, Diamine acetyltransferase inhibitors, Dihydroceramide delta 4 desaturase inhibitors, Dihydroorotate dehydrogenase inhibitors, Dipeptidyl peptidase (DPP)I inhibitors, DPP IV inhibitors, DNA binding protein Ikaros inhibitors, DNA methyltransferase inhibitors, DNA polymerase inhibitors, Dopamine D2 receptor partial agonists, Dopamine D3 receptor partial agonists, Dopamine D4 receptor partial agonists, Dopamine D2 receptor agonists, DYRK-1 alpha protein kinase inhibitors, Ectonucleotide pyrophosphatase-PDE-2 inhibitors, EGFR tyrosine kinase receptor inhibitors, EGR1 gene inhibitors, Elongation factor 2 inhibitors, Endoglin inhibitors, Endoplasmin inhibitors, Endosialin modulators, Endostatin modulators, Endothelin ET-A receptor antagonists, Endothelin ET-B receptor antagonists, Endothelial nitric oxide synthase stimulators, Enolase 1 inhibitors, Enteropeptidase inhibitors, Eotaxin 2 ligand inhibitors, eotaxin ligand inhibitors, EP4 prostanoid receptor antagonists or agonists, EP4 prostanoid receptor antagonists, Epidermal growth factor (EGF) receptor antagonists, EGF modulators, Epoxide hydrolase inhibitors, Erythropoietin receptor antagonists or agonists, Exportin 1 inhibitors, Extracellular matrix protein modulators, FIFO ATP synthase modulators, Facilitated glucose transporter-1 modulators, Factor IIa antagonists, Factor XIIa antagonists, Farnesoid X receptor (FXR) agonists and modulators, Fatty acid synthase inhibitors, fecal microbiota transplantations (FMT), fibroblast activation protein (FAP) inhibitors, Fibroblast growth factor (FGF) receptor agonists and antagonists, FGF-2 ligand inhibitors, FGF1 receptor agonists and antagonists, FGF2 receptor antagonists, FGF3 receptor antagonists, FGF19 gene stimulators, FGF-15 ligands or modulators, FGF-19 ligands or modulators, FGF-21 ligands or modulators, FK506 binding protein inhibitors, FK506 binding protein-10 inhibitors, FK506 binding protein-12 modulators, Flt3 tyrosine kinase inhibitors, Focal adhesion kinase inhibitors, Folate antagonists or agonists, Folate receptor beta antagonists, FP prostanoid receptor antagonists, Fractalkine ligand inhibitors, Free fatty acid receptor 1, 2, and/or 3 agonists, free fatty acid receptor 2 antagonists, Frizzled-5 receptor agonists, Frizzled-8 receptor agonists, Fyn tyrosine kinase inhibitors, G-protein coupled bile acid receptor 1 agonists, G protein coupled receptor 15 antagonists, G-protein beta subunit inhibitors, G-protein coupled receptor (GPCR) 35, 44, 84, 119, 120 modulators, GPCR 44, 87 antagonists, GABA A receptor modulators, GABA A receptor alpha-2 subunit modulators, GABA A receptor alpha-3 subunit modulators, Galanin GAL2 receptor agonists, Galectin-3 inhibitors, Gastric inhibitory polypeptide receptor (GIP-R) agonists and modulators, GATA 3 transcription factor inhibitors, GDNF family receptor alpha like agonists, GHR gene inhibitors, Glucagon-like peptide (GLP) 1 agonists, GLP 2 agonists, GLP 1 receptor modulators, Glucocorticoid agonists or antagonists, Glucocorticoid induced leucine zipper stimulators, Glucokinase stimulators, Glucose 6-phosphate 1-dehydrogenase inhibitors, Glutaminyl peptide cyclotransferase inhibitors, Glutaredoxin 1 modulators, Glutathione dependent PGD synthase inhibitors, Glycoprotein Ib (GPIb) antagonists, GM-CSF receptor antagonists or modulators, GMP synthetase inhibitors, GNRH receptor modulators, GP IIb IIIa antagonists, GPCR modulators, GPR40 agonists, GPR84 antagonists, GroEL protein 2 inhibitors, GroEL protein 2 inhibitors, Growth hormone ligands, Growth hormone receptor agonists, Growth regulated protein alpha ligand inhibitors, guanylate cyclase receptor agonists, Guanylate cyclase stimulators, Heat shock protein inhibitors, H+ K+ ATPase inhibitors, Hedgehog (Hh) modulators, Hh protein inhibitors, Heme oxygenase 1 modulators, Hepatitis B structural protein inhibitors, Hepatitis C virus NS3 protease inhibitors, Hepatitis C virus protein NS5A inhibitors, Hepatocyte nuclear factor 4 alpha modulators (HNF4A), Hepatocyte growth factor modulators and antagonists, hypoxia inducible factor (HIF) prolyl hydroxylase inhibitors, HIF prolyl hydroxylase-2 inhibitors, High mobility group protein B1 inhibitors, Histamine H1 receptor antagonists, Histamine H4 receptor agonists, Histamine H4 receptor antagonists, Histamine H4 receptor modulators, Histone deacetylase (HDAC) inhibitors, HDAC-1 inhibitors, HDAC-2 inhibitors, HDAC-3 inhibitors, HDAC-6 inhibitors, H+ K+ ATPase inhibitors, HIV-1 gp120 protein inhibitors, HLA antigen modulators, HLA class II antigen DQ-2 alpha modulators, HLA class II antigen DR-1 beta inhibitors, HLA class II antigen inhibitors, HLA class II antigen modulators, HMG CoA reductase inhibitors, Homeodomain interacting kinase 2 (HIPK2) inhibitors, Hormone sensitive lipase stimulators, HSD17B3 gene modulators, HSD17B13 gene inhibitors, Hsp 70 family inhibitors and stimulators, Hsp 90 inhibitors, Hyaluronidase stimulators, Hydrolase inhibitors, Hypoxia inducible factor (HIF) modulators, HIF-1 inhibitors, HIF-1 alpha modulators and stimulators, HIF-2 alpha inhibitors, ICAM1 gene inhibitors, ICE inhibitors, interferon beta (IFNB) gene stimulators, Insulin-like growth factor 1 (IGFI) gene inhibitors, IgG receptor FcRn large subunit p51 antagonists, IgG receptor FcRn large subunit p51 modulators, I-kappa B kinase inhibitors, I-kappa B kinase beta inhibitors, IK potassium channel inhibitors, Interleukin (IL)-1 antagonists, IL-2 agonists or antagonists, IL-3 antagonists, IL-4 agonists or antagonists, IL-5 antagonists, IL-6 agonists or antagonists, IL-7 receptor antagonists, IL-8 antagonists, IL-10 antagonists or agonists, IL-11 agonists, IL-12 antagonists, IL-13 antagonists, IL-15 antagonists, IL-17, IL17A, and IL17B agonists or antagonists, IL-18 antagonists, IL-21 antagonists, IL-22 agonists or antagonists, IL-23 antagonists, IL-1 beta ligand modulators, IL-23A inhibitors, IL-31 receptor modulators and antagonists, IL-36 inhibitors, IL-6 neutralizing human antibodies, IL-1 receptor accessory protein inhibitors, IL-18 receptor accessory protein antagonists, IL-2 receptor alpha subunit inhibitors, IL-2 receptor alpha subunit stimulators, Interleukin ligands, IL-1 alpha ligand inhibitors, IL-1 ligand inhibitors, IL-1 beta ligand inhibitors and modulators, IL-1 beta ligands, interleukin ligand inhibitors, IL-2 ligands, IL-4 ligands, IL-4 ligand inhibitors, IL-6 ligand inhibitors, IL-8 ligand inhibitors, IL-10 ligands, IL-13 ligand inhibitors, IL 17 ligand inhibitors, IL 17A ligand inhibitors and modulators, IL-17F ligand inhibitors, IL 18 ligand inhibitors, Interleukin-22 ligands, IL-29 ligands, IL-33 ligand inhibitors, IL-1 like receptor inhibitors, Ileal sodium bile acid cotransporter inhibitors, immunoglobulin (Ig) agonists or antagonists, IgE antagonists and modulators, Immunoglobulin Fc receptor modulators, IgG agonists, IgG1 agonists and antagonists, IgG2 antagonists and modulators, Immunoglobulin gamma Fc receptor antagonists, Immunoglobulin gamma Fc receptor II modulators, Immunoglobulin gamma Fc receptor IIB antagonists, Immunoglobulin kappa modulators, Immunoglobulin like domain receptor 2 antagonists, IgM antagonists, Inducible nitric oxide synthase inhibitors (iNOS inhibitors), Inducible T-cell co-stimulator inhibitors, Inosine monophosphate dehydrogenase inhibitors, Insulin ligands, Insulin ligand agonists, Insulin receptor agonists, Insulin receptor substrate-1 inhibitors, Insulin sensitizers, integrin antagonists and modulators, Integrin alpha-1/beta-1 antagonists, Integrin alpha-4/beta-1 antagonists, Integrin alpha-V/beta-1 antagonists, Integrin alpha-V/beta-3 antagonists, Integrin alpha-V/beta-6 antagonists, Integrin alpha-V/beta-8 modulators, integrin alpha-4/beta-7 antagonists, Integrin alpha-9 antagonists, Interferon (IFN) alpha ligands, IFN alpha ligand inhibitors and modulators, IFN omega ligand inhibitors, IFN beta ligands, IFN beta ligand inhibitors, IFN gamma ligands, IFN gamma receptor 1 agonists, IFN gamma receptor antagonists, IFN type I receptor antagonists, interleukin-1 receptor-associated kinase 4 (IRAK4) inhibitors, IRE1 protein kinase inhibitors, Itk tyrosine kinase inhibitors, Janus Kinase (JAK) inhibitors and modulators, JAK3 gene inhibitors, JAK1 inhibitors, JAK2 inhibitors, JAK3 inhibitors, Jun N terminal kinase inhibitors, Jun N terminal kinase-1 inhibitors, Kallikrein inhibitors, Kallikrein 2 inhibitors, Kallikrein 7 inhibitors, KCNA voltage-gated potassium channel-3 inhibitors, KCNA voltage-gated potassium channel-3 modulators, KCNN potassium channel-4 inhibitors, KCNN4 gene inhibitors, Kelch like ECH associated protein 1 modulators, Ketohexokinase (KHK) inhibitors, Kit tyrosine kinase inhibitors, Klotho beta stimulators, lactoferrin stimulators, LanC like protein 2 stimulators, LanC like protein 2 modulators, Lck tyrosine kinase inhibitors, LDHA gene inhibitors, LDL receptor related protein-1 stimulators, LDL receptor related protein-6 inhibitors, LDL receptor related protein-6 stimulators, Lectin mannose binding protein inhibitors, leukocyte elastase inhibitors, Leukocyte Ig like receptor A4 modulators, leukocyte proteinase-3 inhibitors, Leukotriene receptor antagonists, Leukotriene A4 hydrolase inhibitors, Leukotriene BLT receptor antagonists, Leukotriene D4 antagonists, 5-Lipoxygenase activating protein inhibitors, 5-Lipoxygenase inhibitors, Lipoxygenase modulators, Lipoprotein lipase inhibitors, LITAF gene inhibitors, Liver X receptor agonists and antagonists, Liver X receptor alpha inverse agonists, Liver X receptor beta inverse agonists, LPL gene stimulators, Lymphocyte function antigen-3 receptor antagonists, Lyn tyrosine kinase inhibitors, Lyn tyrosine kinase stimulators, Lysophosphatidate-1 receptor antagonists, Lysyl oxidase homolog (LOXL) 2 inhibitors, LXR inverse agonists, macrophage-drug conjugates (MDC), Macrophage inflammatory protein (MIP) 2 alpha inhibitors, MIP 2 beta inhibitors, MIP 3 alpha ligand inhibitors, Macrophage mannose receptor 1 modulators, Macrophage migration inhibitory factor inhibitors, MAdCAM inhibitors, MAdCAM modulators, MALT protein 1 inhibitors, Mannan-binding lectin serine protease-2 inhibitors, MAP kinase inhibitors, MAP kinasekinase 4 inhibitors, MAP kinase modulators, MAP3K2 gene inhibitors, MAPKAPK2 inhibitors, MAPKAPK5 inhibitors, Matrix extracell phosphoglycoprotein modulators, Matrix metalloprotease inhibitors, MCH receptor-1 antagonists, MCL1 gene inhibitors, MEK protein kinase inhibitors, MEK-1 protein kinase inhibitors, MEK-2 protein kinase inhibitors, MEKK-5 protein kinase inhibitors, melanin concentrating hormone (MCH-1) antagonists, melanocortin agonists, Melanocortin MC1 receptor agonists, Melanocortin MC3 receptor agonists, Melanocortin receptor agonists, Membrane copper amine oxidase inhibitors, Metalloprotease-1 inhibitors, Metalloprotease-2 inhibitors, Metalloprotease-9 inhibitors, Metalloprotease-9 stimulators, methylprednisolone, Methionine aminopeptidase-2 inhibitors, Methyl CpG binding protein 2 modulators, microbiome-targeting therapeutics, MicroRNA-132 (miR-132) antagonists, MicroRNA-21 (miR-21) inhibitors, Midkine ligand inhibitors, Mineralocorticoid receptor antagonists and modulators, Mitochondrial uncouplers, Mitochondrial 10 kDa heat shock protein stimulators, Mitochondrial pyruvate carrier 2 inhibitors, Mitochondrial pyruvate carrier inhibitors, Mixed lineage kinase-3 inhibitors, MKL myocardin like protein inhibitors, MNK protein kinase inhibitors, Monocarboxylate transporter inhibitors, Monocyte macrophage differentiation inhibitors, Motile sperm domain protein 2 inhibitors, MST-1 protein kinase inhibitors, mTOR complex 1 inhibitors, mTOR complex 2 inhibitors, mTOR inhibitors, Myelin basic protein stimulators, Myeloperoxidase inhibitors, Myosin 2 inhibitors, N-formyl peptide receptor antagonists, NACHT LRR PYD domain protein 3 (NLRP3) inhibitors, NAD ADP ribosyltransferase stimulators, NAD-dependent deacetylase sirtuin stimulators, NAD-dependent deacetylase sirtuin-1 stimulators, NADPH oxidase inhibitors, NADPH oxidase 1 inhibitors, NADPH oxidase 4 inhibitors, NAMPT gene inhibitors, natriuretic peptide receptor C agonists, neuregulin-4 ligands, Neuropilin 2 modulators, Neutral endopeptidase inhibitors, NF kappa B inhibitor stimulators, NFAT gene inhibitors, NFE2L2 gene inhibitors, NFE2L2 gene stimulators, Nicotinic acetylcholine receptor antagonists, Nicotinic acid receptor 1 agonists, Nicotinamide phosphoribosyltransferase inhibitors, NK cell receptor modulators, NK1 receptor antagonists, NKG2 A B activating NK receptor antagonists, NKG2 D activating NK receptor antagonists, NLR family member X1 stimulators, NLRP3 inhibitors, NMDA receptor epsilon 2 subunit inhibitors, NOD2 gene modulators, Non receptor tyrosine kinase TYK2 antagonists, NOX4 gene inhibitors, NUAK SNF1-like protein kinase 1 inhibitors, Nuclear erythroid 2-related factor 2 stimulators, Nuclear factor kappa (NFK) B inhibitors and modulators, Nuclear factor kappa B p105 inhibitors, nuclear hormone receptor modulators, Nuclear pore complex protein modulators, Nuclear receptor modulators, Nuclease stimulators, Nucleoside reverse transcriptase inhibitors, Nucleosome assembly protein 1 like-4 inhibitors, Oncostatin M receptor modulators, Oncostatin M receptor subunit beta inhibitors, opioid receptor antagonists, Opioid growth factor receptor agonists, Opioid receptor delta, kappa, and mu antagonists, Opioid receptor sigma antagonist 1, Orphan nuclear receptor antagonists, Osteoclast differentiation factor antagonists, Osteoclast differentiation factor ligand inhibitors, Oxidoreductase inhibitors, OX40 ligand inhibitors, OX-40 receptor antagonists and modulators, Oxyntomodulin ligands, PGE1 agonists, P-Glycoprotein inhibitors, P-selectin glycoprotein ligand-1, 14-3-3 protein eta inhibitors, P2X3 purinoceptor antagonists, P2X7 purinoceptor agonists and modulators, P2Y6 purinoceptor modulators, P2Y13 purinoceptor stimulators, p38 MAP kinase alpha inhibitors, p38 MAP kinase inhibitors, p53 tumor suppressor protein stimulators, PACAP type I receptor agonists, Pan cathepsin inhibitors, Parathyroid hormone ligand inhibitors, PARP modulators, PDE 1 inhibitors, PDE 3 inhibitors, PDE 4 inhibitors, PDE 4b inhibitors, PDE 5 inhibitors, PDGF-B ligand inhibitors, PDGF receptor agonists, PDGF receptor alpha antagonists, PDGF receptor beta antagonists and modulators, PEGylated long-acting glucagon-like peptide-1/glucagon (GLP-1R/GCGR) receptor dual agonists, Pellino homolog 1 inhibitors, Peptidyl-prolyl cis-trans isomerase A inhibitors, Peptidyl-prolyl cis-trans isomerase D inhibitors, PERK gene inhibitors, PGI2 agonists, PGD2 antagonists, Phenylalanine hydroxylase stimulators, Phosphatidylinositol 3 kinase subunit 3 inhibitors, Phosphatonin receptor agonists, Phosphoinositide 3-kinase inhibitors, Phosphoinositide-3 kinase alpha, delta, and gamma inhibitors, Phospholipase A2 inhibitors, Phospholipase C inhibitors, Phosphoric diester hydrolase inhibitors, Phosphorylase inhibitors, Plasma retinol binding protein inhibitors, Plasminogen activator inhibitor 1 inhibitors, Plasmin stimulators, Platelet activating factor receptor antagonists, Plexin domain containing protein stimulators, PNPLA3 gene inhibitors and modulators, Potassium channel inhibitors PPAR agonists, PPAR alpha/delta agonists, PPAR delta agonists, PPAR gamma agonists and modulators, PRKAA2 gene stimulators, Programmed cell death ligand (PDL) 1 modulators, Programmed cell death protein 1 modulators, Programmed cell death protein 1 stimulators, Proprotein convertase PC9 inhibitors, Prostacyclin (PGI2) agonists, Prostaglandin D synthase stimulators, Prostanoid receptor antagonists, Protease-activated receptor-2 antagonists, Proteasome beta-8 subunit modulators, Proteasome inhibitors, Protein arginine deiminase inhibitors, Protein arginine deiminase IV inhibitors, Protein C activators, Protein cereblon modulators, protein fimH inhibitors, Protein kinase C theta inhibitors, Protein kinase inhibitors and modulators, Protein kinase C theta inhibitors, Protein MB21D1 inhibitors and modulators, Protein NOV homolog modulators, P-selectin glycoprotein ligand-1 inhibitors, Protein tyrosine kinase inhibitors, Protein tyrosine phosphatase beta inhibitors, Protein tyrosine phosphatase-1B inhibitors, Protein tyrosine phosphatase-2C inhibitors, Protein tyrosine phosphatase 1E inhibitors, P-selectin glycoprotein ligand-1 stimulators, PTGS2 gene inhibitors, PurH purine biosynthesis protein inhibitors, QSK serine threonine protein kinase inhibitors, Ras gene inhibitors, Reactive oxygen species modulator inhibitors, Relaxin receptor modulators, Relaxin receptor 2 modulators, Renin inhibitors, Resistin ligand inhibitors, Resistin/CAP1 (adenylyl cyclase associated protein 1) interaction inhibitors, Retinoic acid receptor agonists, Retinoic acid receptor gamma antagonists and inverse agonists, Retinoid receptor agonists, Retinoid X receptor agonists and modulators, Retinoid Z receptor gamma agonists and antagonists, Ret tyrosine kinase receptor inhibitors, Rev protein modulators, Rho associated protein kinase inhibitors, Rho associated protein kinase 1 inhibitors, Rho associated protein kinase 2 inhibitors, Rhomboid family member 2 inhibitors, Ribonuclease P inhibitors, RIP-1 kinase inhibitors, RIP-2 kinase inhibitors, RNA polymerase inhibitors, Seprase inhibitors, Serine threonine protein kinase TBK1 inhibitors, Serine threonine protein kinase TBK1 modulators, Serine threonine SNF1 like kinase 2 inhibitors, SERPINHI gene inhibitors, Serum amyloid A protein modulators, Serum amyloid P stimulators, Signal transducer CD24 modulators, Signal transduction inhibitors, SLC22A12 inhibitors, SMAD inhibitors, SMAD-3 inhibitors, Smoothened receptor antagonists, S-nitrosoglutathione reductase (GSNOR) enzyme inhibitors, Sodium channel inhibitors, Sodium glucose transporter-1 inhibitors, Sodium glucose transporter-2 inhibitors, Solute carrier family inhibitors, Somatostatin receptor agonists, Sphingolipid delta 4 desaturase DES1 inhibitors, Sphingosine kinase 1 inhibitors, Sphingosine kinase 2 inhibitors, Sphingosine 1 phosphate phosphatase modulators, sphingosine 1 phosphate phosphatase 1 stimulators, sphingosine-1-phosphate receptor-1 agonists, sphingosine-1-phosphate receptor-5 agonists, sphingosine-1-phosphate receptor-1 antagonists, sphingosine-1-phosphate receptor-1 modulators, Sphingosine-1-phosphate receptor-3 modulators, Sphingosine-1-phosphate receptor-4 modulators, Sphingosine-1-phosphate receptor-5 modulators, Src tyrosine kinase inhibitors, SREBP transcription factor inhibitors, SREBP transcription factor 1 inhibitors, SREBP transcription factor 2 inhibitors, STAT inhibitors, STAT3 gene inhibitors, STAT-1 inhibitors and modulators, STAT-3 inhibitors and modulators, STAT-5 inhibitors, STAT-6 inhibitors, Stearoyl CoA desaturase-1 inhibitors, stem cell antigen-1 inhibitors, Stimulator of interferon genes protein inhibitors, STK25 inhibitors, Stress induced secreted protein 1 stimulators, superoxide dismutase modulators, Superoxide dismutase stimulators, Suppressor of cytokine signalling-1 stimulators, Suppressor of cytokine signalling-3 stimulators, SYK inhibitors, Syndecan-1 inhibitors, TACE inhibitors, TAK1 binding protein modulators, Talin modulators, Taste receptor type 2 agonists, T-box transcription factor TBX21 modulators, T-cell differentiation antigen CD6 inhibitors, T cell receptor modulators, T cell receptor antagonists, T-cell surface glycoprotein CD1a inhibitors, T-cell surface glycoprotein CD8 inhibitors, T cell surface glycoprotein CD28 inhibitors, T-cell surface glycoprotein CD8 modulators, T cell surface glycoprotein CD28 stimulators, T-cell transcription factor NFAT modulators, Tec tyrosine kinase inhibitors, Telomerase stimulators, Tenascin modulators, TERT gene modulators, TGF-beta activated kinase-1 inhibitors, TGF-beta activation modulators, TGF beta agonists, TGF beta ligand inhibitors, TGF beta 1 ligand inhibitors, TGF beta 3 ligand inhibitors, TGF beta 1 gene inhibitors, TGF beta 1 ligand modulators, TGF beta receptor antagonists, TGF beta receptor antagonists, TGF-beta type II receptor antagonists, TGFB1 gene inhibitors, Thioredoxin reductase inhibitors, Thrombomodulin stimulators, Thromboxane A2 antagonists, Thromboxane A2 receptor antagonists, Thromboxane synthesis inhibitors, Thymic stromal lymphopoietin ligand inhibitors, Thymic stromal lymphopoietin ligand modulators, Thymic stromal lymphopoietin receptor modulators, Thymulin agonists, Thyroid hormone receptor agonists, Thyroid hormone receptor beta agonists, tissue transglutaminase inhibitors, Toll-like receptor (TLR)-2 antagonists, TLR-3 antagonists, TLR-4 antagonists, TLR-7 antagonists and modulators, TLR-8 antagonists, TLR-9 antagonists and agonists, TLR modulators, TNF alpha ligand agonists and antagonists, TNF ligand agonists and antagonists, TNF binding agents, TNF gene inhibitors, TNFSF11 gene inhibitors, Topoisomerase II inhibitors, TPL-2 inhibitors, Transaminase stimulators, Transcription factor modulators, Transcription factor p65 inhibitors, Transcription factor RelB inhibitors, Transferrin modulators, Transforming growth factor β (TGF-β), Transforming growth factor β activated Kinase 1 (TAK1), Transglutaminase inhibitors, Transthyretin modulators, TrkA receptor antagonists, Trk tyrosine kinase receptor inhibitors, TRP cation channel A1 inhibitors, TRP cation channel C5 inhibitors, TRP cation channel C6 inhibitors, Tryptophan 5-hydroxylase-1 inhibitors, Tryptophanase inhibitors, Tubulin binding agents, Tumor necrosis factor ligand inhibitors, Tumor necrosis factor ligand 13 inhibitors, Tumor necrosis factor 15 ligand inhibitors, tumor necrosis factor 14 ligand modulators, Tumor necrosis factor 13C receptor antagonists, Tumor necrosis factor 14 ligand inhibitors, Tyk2 tyrosine kinase inhibitors, Type I IL-1 receptor antagonists, Type I TNF receptor antagonists, Type II TNF receptor antagonists, Type II TNF receptor modulators, Tyrosine kinase receptor inhibitors, Tyrosine kinase receptor modulators, Ubiquitin ligase modulators and stimulators, Ubiquitin thioesterase-30 inhibitors, Uncoupling protein modulators, Unspecified cell adhesion molecule inhibitors, Unspecified GPCR agonists, Unspecified GPCR modulators, Unspecified growth factor receptor antagonists, Urate anion exchanger 1 inhibitors, vanilloid VR1 agonists, Vanilloid VR1 antagonists, Vasopressin Via receptor antagonists, VDR agonists, VEGF receptor antagonists, VEGF receptor modulators, VEGF-1 receptor antagonists, VEGF-2 receptor antagonists, VEGF-3 receptor antagonists, VEGF-2 receptor modulators, VEGF-B ligand inhibitors, Vimentin inhibitors, VIP 1 receptor agonists, VIP 2 receptor agonists, Vitamin D3 receptor agonists, Vitamin D3 receptor modulators, Vitamin K dependent protein C stimulators, WNT modulators, Wnt ligand inhibitors, Wnt 5A ligand inhibitors, Xanthine oxidase inhibitors, X-linked inhibitor of apoptosis protein inhibitors, XPO1 gene modulators, YAP/TAZ modulators, YSK-4 protein kinase inhibitors, Zap70 tyrosine kinase inhibitors, Zinc finger binding protein Aiolos inhibitors, zonulin inhibitors.

### Rheumatoid Arthritis

In some embodiments, a compound of the disclosure, or a pharmaceutically acceptable salt thereof, is co-administered with one or more agents useful for the treatment and/or prophylaxis of a rheumatological condition.

In some embodiments, a compound of the disclosure, or a pharmaceutically acceptable salt thereof, is co-administered with one or more agents useful for the treatment and/or prophylaxis of rheumatoid arthritis. Non-limiting examples of such agents include disease-modifying antirheumatic drugs (DMARDS), such as hydroxychloroquine, sulfasalazine, methotrexate, and leflunomide; TNF inhibitors (e.g., etanercept, adalimumab, infliximab, golimumab, certolizumab pegol), T cell costimulatory inhibitor, (e.g., abatacept), IL-6 receptor inhibitors (e.g., tocilizumab, sarilumab), anti-CD20 antibody (e.g., rituximab); and JAK inhibitors (e.g., tofacitinib, baricitinib, upadacitinib); NSAIDs, such as ibuprofen, naproxen, and diclofenac; COX-2 inhibitor, such as celecoxib and etoricoxib; steroids and corticosteroids, such as prednisolone and cortisone; and biological agents known for treatment and/or prophylaxis of such conditions, including for example etanercept (e.g., ENBREL), infliximab (e.g., REMICADE), adalimumab (e.g., HUMIRA), anakinra (e.g., KINARET), abatacept (ORENCIA), rituximab (e.g., RITUXAN), certolizumab (e.g., CIMZIA), golimumab (e.g., SIMPONI), and tocilizumab (e.g., ACTEMRA). In some embodiments, a compound of the disclosure is administered with two additional thereapeutic agents useful for the treatment and/or prophylaxis of a rheumatological condition. In some embodiments, agents useful for the treatment and/or prophylaxis of a rheumatological condition include a compound of the disclosure and two additional therapeutic agents, such as methotrexate+leflunomide, methotrexate+sulfasalazine, methotrexate+cyclosporine, methotrexate+hydroxychloroquine and triple therapy treatments hydroxychloroquine+sulfasalazine+methotrexate, hydroxychloroquine+sulfasalazine+leflunomide.

### Lupus

In some embodiments, a compound of the disclosure, or a pharmaceutically acceptable salt thereof, is co-administered with one or more agents useful for the treatment and/or prophylaxis of systemic lupus erythematosus (SLE) or lupus nephritis (LN). Non-limiting examples of such agents include immunosuppressive drugs that inhibit activity of the immune system and agents approved for treatment of SLE, such as hydroxychloroquine, steroids and corticosteroids (e.g., prednisone, methylprednisolone), belimumab, azathioprine, methotrexate, cyclophosphamide, mycophenolate and mycophenolate mofetil, cyclosporine, leflunomide, voclosporin, abatacept, anifrolumab, rituximab, NSAIDS, such as naproxen sodium and ibuprofen, antimalarial drugs, such as hydroxychloroquine, calcineurin inhibitors, and tacrolimus.

In some embodiments, a compound of the disclosure, or a pharmaceutically acceptable salt thereof, is co-administered with two or more agents useful for the treatment of LN, such as prednisone+mycophenolic acid analogs, prednisone+mycophenolic acid sodium prednisone+cyclophosphamide, prednisone+tacrolimus, prednisone+voclosporin, prednisone+belimumab+mycophenolic acid analogs, prednisone+belimumab+cyclophosphamide, prednisone+rituximab.

In further embodiments, a compound of the disclosure, or a pharmaceutically acceptable salt thereof, is co-administered with two or more agents useful for the treatment of LN, such as prednisone+mycophenolic acid analogs, prednisone+mycophenolic acid sodium, prednisone+Azathioprine, prednisone+Tacrolimus, prednisone+cyclosporine, prednisone+mizoribine.

### Osteoarthritis

In some embodiments, a compound of the disclosure, or a pharmaceutically acceptable salt thereof, is co-administered with one or more agents useful for the treatment and/or prophylaxis of osteoarthritis (OA). Non-limiting examples of such agents include nonsteroidal anti-inflammatory drugs (NSAIDs), topical capsaicin, intraarticular glucocorticoid injections, acetaminophen, duloxetine, tramadol, and injectable corticosteroids such as methylprednisolone acetate, triamcinolone acetate, betamethasone acetate and betamethasone sodium phosphate, triamcinolone hexacetonide, and dexamethasone.

### Ulcerative Colitis

In some embodiments, a compound of the disclosure, or a pharmaceutically acceptable salt thereof, is co-administered with one or more agents useful for the treatment and/or prophylaxis of a gastroenterologic condition such as ulcerative colitis (UC) or Crohn's disease (CD). Non-limiting examples of such agents include infliximab, adalimumab, golimumab, vedolizumab, tofacitinib, ustekinumab, natalizumab, mesalamine, diazo-bonded 5-ASA, sulfasalazine, balsalazide, olsalazine, corticosteroids such as budesonide, hydrocortisone, methylprednisolone, and prednisone; immunosuppressants or immunomodulators such as azathioprine and 6-mercaptopurine, cyclosporine, and methotrexate.

### Pulmonology

In some embodiments, a compound of the disclosure, or a pharmaceutically acceptable salt thereof, is co-administered with one or more agents useful for the treatment and/or prophylaxis of a pulmonologic condition, such as idiopathic pulmonary fibrosis (IPF) or interstitial lung disease (ILD). Non-limiting examples of such agents include nitendanib, pirfenidone, corticosteroids such as prednisone, other rheumatologic drugs, including mycophenolate (e.g., CellCept®), azathioprine (e.g., Imuran®), leflunomide (e.g., ARAVA®), rituximab (e.g., RITUXAN®), cyclophosphamide (e.g., CYTOXAN®), tacrolimus (e.g., PROGRAF®), medications that reduce stomach acid, such as H-2-receptor antagonists or proton pump inhibitors such as lansoprazole (e.g., PREVACID®24HR), omeprazole (e.g., Prilosec OTC) and pantoprazole (e.g., PROTONIX®).

### Heptatology and Nephrology

In some embodiments, a compound of the disclosure, or a pharmaceutically acceptable salt thereof, is co-administered with one or more agents useful for the treatment and/or prophylaxis of a heptatologic or nephrologic condition, such as NAFLD, NASH, DKD, or CKD. Non-limiting examples of such agents include metformin, sodium-glucose cotransporter-2 inhibitor (SGLT2i), drug therapy for glycemic control, DPP-4 inhibitor, insulin, sulfonylurea, TZD (thiazolidinedione), alpha-glucosidase inhibitor, SGLT2 inhibitor (e.g., empagliflozin, canagliflozin, dapaglifloz), glucagon-like peptide-1 receptor agonist (GLP-1 RA) (e.g., lixisenatide, liraglutide, semaglutide, exenatide, albiglutide, dulaglutide), DPP-4 inhibitors (e.g., saxagliptin, alogliptin, sitagliptin, linagliptin), one or more agents used to treat high blood pressure such as angiotensin-converting enzyme (ACE) inhibitors and angiotensin 2 receptor blockers (ARBs), agents supportive of weight loss or for control of blood sugar, cholesterol-lowering drugs (e.g., statins), finerenone, and agents for treatment of diabetes mellitus, such as alpha-glucosidase inhibitors (e.g., acarbose, miglitol, voglibose).

### Dermatology

In some embodiments, a compound of the disclosure, or a pharmaceutically acceptable salt thereof, is co-administered with one or more agents useful for the treatment and/or prophylaxis of a dermatologic condition, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), topical antimicrobials and antiseptics, cyclosporine, methotrexate, mycophenolate mofetil, interferon gamma, phosphodiesterase 4 (PDE4) inhibitor such as crisaborole, JAK inhibitor (e.g., ruxolitinib, upadacitinib, abrocitinib), systemic glucocorticoids (e.g., prednisone), dupilumab, and anti-IL-13 antibody (e.g., tralokinumab).

### Synthesis

In some embodiments, a method of synthesizing Formula (I) and/or Formula (II) is provided. In some embodiments, Intermediate (I-2) and compound of Formula (II) are formed according to the following synthetic scheme:

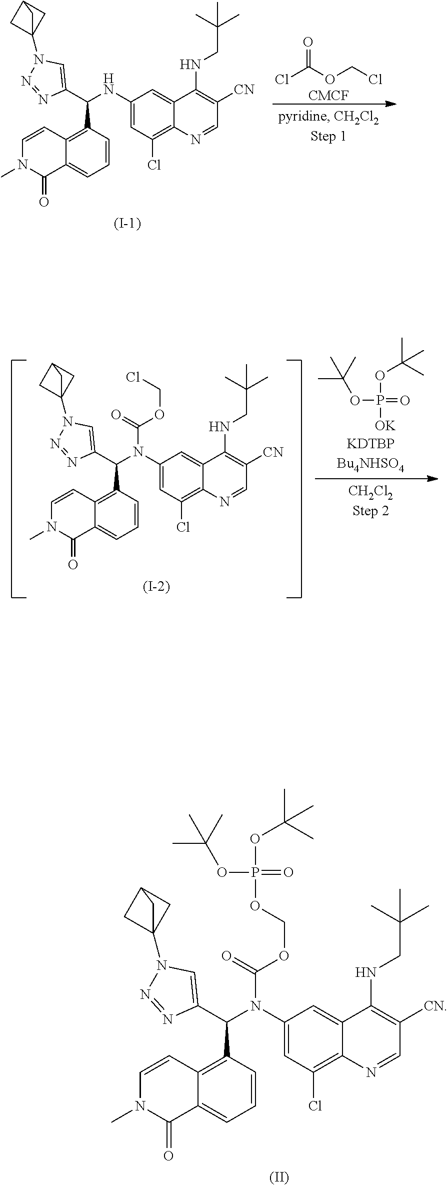

In some embodiments, a process of preparing a compound of Intermediate (I-2):

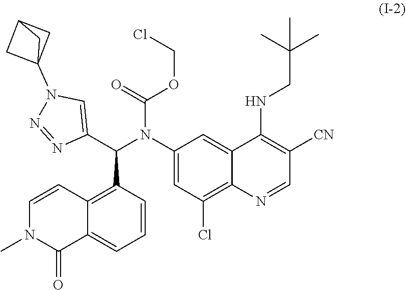 [SMILES: Cn1ccc2c([C@@H](c3cn(C45CC(C4)C5)nn3)N(C(=O)OCCl)c3cc(Cl)c4ncc(C#N)c(NCC(C)(C)C)c4c3)cccc2c1=O]

comprises reacting a compound of Intermediate (I-1):

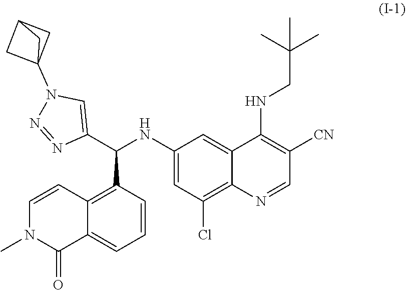 [SMILES: Cn1ccc2c([C@H](Nc3cc(Cl)c4ncc(C#N)c(NCC(C)(C)C)c4c3)c3cn(C45CC(C4)C5)nn3)cccc2c1=O]

or a salt thereof, with a base and chloromethyl chloroformate. In some embodiments, the base comprises a carbonate, metal hydride, or an organic base. In some embodiments, the base is an aromatic base. In some embodiments, the base is pyridine. In some embodiments, the reacting the compound of Intermediate (I-2) is performed in a solvent. In some embodiments, the solvent comprises a halogenated solvent. In some embodiments, the solvent comprises dichloromethane.

Intermediate (I-1) can be combined with solvent, such as dichloromethane, a base, such as pyridine, and choloromethyl chloroformate to yield Intermediate (I-2). Other exemplary solvents that can be used include, but are not limited to ethers (such as tetrahydrofuran, 2-methyltetrahydrofuran, tert-butyl methyl ether), polar aprotic solvents (such as N,N-dimethylformamide, N,N-dimethylacetamide, N-methyl-2-pyrrolidone), halogenated solvents (such as dichloromethane, 1,2-dichloroethane, chlorobenzene) and hydrocarbons (such as toluene, n-heptane). Other exemplary bases that can be used include, but are not limited to carbonates (such as lithium, sodium, potassium, cesium carbonate), metal hydrides (such as sodium hydride, potassium hydride), hindered alkoxides (such as sodium tert-butoxide, lithium tert-butoxide), and organic bases (such as 1,8-diazabicyclo(5.4.0)undec-7-ene, 1,5-diazabicyclo(4.3.0)non-5-ene, 2,6-lutidine). A suitable temperature for the reaction can range from −30 to 60° C.

In some embodiments, a process of preparing a compound of Formula (II):

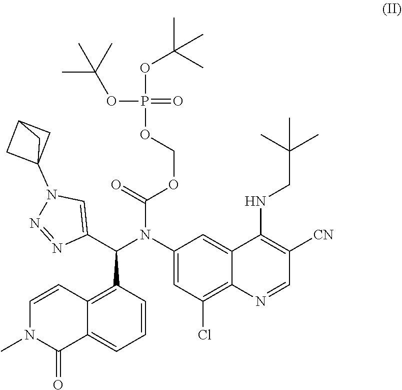 [SMILES: Cn1ccc2c([C@@H](c3cn(C45CC(C4)C5)nn3)N(C(=O)OCOP(=O)(OC(C)(C)C)OC(C)(C)C)c3cc(Cl)c4ncc(C#N)c(NCC(C)(C)C)c4c3)cccc2c1=O]

comprises reacting a compound of Intermediate (I-2), or a salt thereof, with a phosphate source in the presence of a catalyst. In some embodiments, the phosphate source comprises a di-tert-butylphosphate salt. In some embodiments, the phosphate source comprises potassium di-tert-butylphosphate. In some embodiments, the catalyst comprises a quaternary ammonium salt. In some embodiments, the catalyst comprises tetra-n-butylammonium hydrogen sulfate. In some embodiments, the reacting the compound of Intermediate (I-2) is performed in a solvent. In some embodiments, the solvent comprises a halogenated solvent. In some embodiments, the solvent comprises dichloromethane.

Formula (II) can be formed by combining Intermediate (I-2) in a solvent with a phosphate source in the presence of a catalyst. A phosphate source can include potassium di-tert-butylphosphate or, for instance, sodium di-tert-butylphosphate or cesium di-tert-butylphosphate. Exemplary catalysts that can be used include, without limitation, tetra-n-butylammonium hydrogen sulfate, or quaternary ammonium salts (i.e., tetrabutylammonium chloride, tetra-n-butylammonium bromide, etc.) sodium iodide, and other promoters known in the state of the art to promote Finkelstein-like reactions. Exemplary solvents that can be used include ethers (such as tetrahydrofuran, 2-methyltetrahydrofuran, tert-butyl methyl ether), polar aprotic solvents (such as N,N-dimethylformamide, N,N-dimethylacetamide, N-methyl-2-pyrrolidone), halogenated solvents (such as dichloromethane, 1,2-dichloroethane, chlorobenzene), hydrocarbons (such as toluene, n-heptane), esters (such as ethyl acetate, isopropyl acetate), or combinations of the foregoing optionally with water. A suitable temperature for the reaction can range from 0 to 60° C.

In some embodiments, a process of preparing a compound of Formula I comprises deprotecting a compound of Formula (II), or a salt thereof, with an acid source. In some embodiments, the acid source comprises acetyl chloride, acetic acid, hydrogen chloride, sulfuric acid, phosphoric acid, trifluoroacetic acid, para-toluenesulfonic acid, hydrogen chloride gas, or sources of anhydrous hydrogen chloride. In some embodiments, the acid source comprises an acyl halide. In some embodiments, the acid source comprises an acetyl halide. In some embodiments, the acid source comprises acetyl chloride. In some embodiments, the deprotecting the compound of Formula (II) is performed in a solvent. In some embodiments, the solvent comprises an alcohol solvent. In some embodiments, the solvent comprises methanol.

In some embodiments, a compound of Formula (I) is formed by deprotecting Formula (II), for instance according to the following scheme.

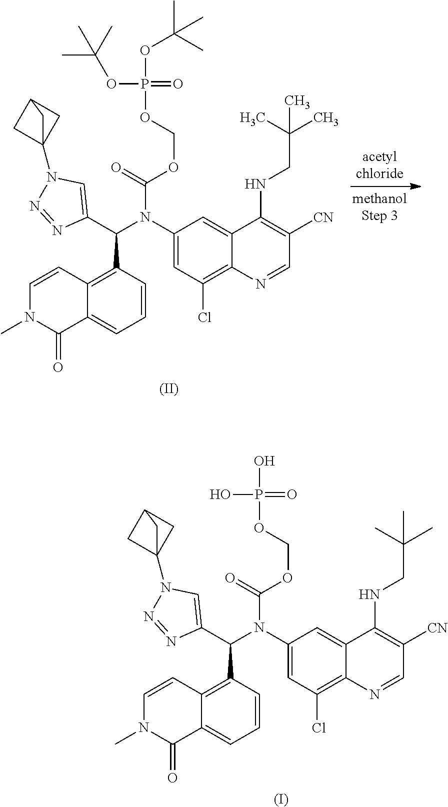 [SMILES: Cn1ccc2c([C@@H](c3cn(C45CC(C4)C5)nn3)N(C(=O)OCOP(=O)(OC(C)(C)C)OC(C)(C)C)c3cc(Cl)c4ncc(C#N)c(NCC(C)(C)C)c4c3)cccc2c1=O ; "(II)" ; Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O ; "(I)"]

Formula (II) can be combined with solvent and an acid source. Examples of acids that can be used as acid source include acetyl chloride acetic acid, concentrated hydrogen chloride, concentrated sulfuric acid, phosphoric acid, trifluoroacetic acid, para-toluenesulfonic acid, hydrogen chloride gas, and sources of anhydrous hydrogen chloride (i.e., acid chloride and alcohol solvent). Solvents that can be used include alcohols (such as methanol, ethanol, 2-propanol), ethers (such as tetrahydrofuran, 2-methyltetrahydrofuran, tert-butyl methyl ether), polar aprotic solvents (such as N,N-dimethylformamide, N,N-dimethylacetamide, N-methyl-2-pyrrolidone), acids (such as acetic acid), halogenated solvents (such as dichloromethane, 1,2-dichloroethane, chlorobenzene), hydrocarbons (such as toluene, n-heptane), and/or combinations of the foregoing with water. A suitable temperature for the reaction can range from 0 to 50° C.

In some embodiments, a process of preparing a compound of Formula I comprises:
(a) reacting a compound of Intermediate (I-1) or a salt thereof, with a base and chloromethyl chloroformate to prepare a compound of Intermediate (I-2);
(b) reacting the compound of Intermediate (I-2) or a salt thereof, with a phosphate source in the presence of a catalyst to prepare a compound of Formula (II); and
(c) deprotecting the compound of Formula (II) or a salt thereof, with an acid source to prepare the compound of Formula I.

In some embodiments, a process of preparing a compound of Formula I comprises:
(a) reacting a compound of Intermediate (I-2) or a salt thereof, with a phosphate source in the presence of a catalyst to prepare a compound of Formula (II); and
(b) deprotecting the compound of Formula (II) or a salt thereof, with an acid source to prepare the compound of Formula I.

In some embodiments, a process of preparing a compound of Formula I comprises reacting a compound of Intermediate (I-1) or a salt thereof, with a base and chloromethyl chloroformate to prepare a compound of Intermediate (I-2). In some embodiments, a process of preparing a compound of Formula I comprises reacting a compound of Intermediate (I-2) or a salt thereof, with a phosphate source in the presence of a catalyst to prepare a compound of Formula (II).

In some embodiments, a process of preparing a compound of Formula (II) comprises:
(a) reacting a compound of Intermediate (I-1) or a salt thereof, with a base and chloromethyl chloroformate to prepare a compound of Intermediate (I-2); and
(b) reacting the compound of Intermediate (I-2) or a salt thereof, with a phosphate source in the presence of a catalyst to prepare the compound of Formula (II).

In some embodiments, a process of preparing a compound of Formula II comprises reacting a compound of Formula (I-1) or a salt thereof, with a base and chloromethyl chloroformate to prepare a compound of Intermediate (I-2).

### EXAMPLES

### Methods

Compounds of Formula I were synthesized according to known methods, such as those disclosed in U.S. Pat. No. 10,947,259.

### X-Ray Powder Diffraction (XRPD)

XRPD patterns were collected with a PANalytical X'Pert PRO MPD diffractometer using an incident beam of Cu Ku radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b.

### Differential Scanning Calorimetry (DSC)

Differential Scanning Calorimetry (DSC) data were collected using a TA Instruments Q2000 differential scanning calorimeter. Temperature calibration was performed using NIST-traceable indium metal. The sample was placed into a T zero aluminum DSC pan, covered with a lid with a hole pierced using a needle. The weight was then accurately recorded. A weighed aluminum pan configured as the sample pan was placed on the reference side of the cell. The sample was heated from 0° C. to 300° C. at 10° C./minute.

### Thermo-Gravimetric Analysis (TGA)

Thermogravimetric Analysis (TGA) data were collected using a TA Instruments Discovery thermogravimetric analyzer. Temperature calibration was performed using nickel and Alumel™. Each sample was placed in an aluminum pan and inserted into the TG furnace. The furnace was heated under a nitrogen purge. The sample was heated from ambient to 300° C. at 10° C./minute.

Thermogravimetric-Mass Spectrometer analysis (TG-MS)

Thermogravimetric-Mass Spectrometer analysis (TG-MS) was used to identify volatile off-gassing and to evaluate sample weight loss as a function of temperature on a Discovery TGA (TA Instruments, New Castle, DE) by loading 1-10 mg of material onto a weigh pan and heating the sample to fully desolvate the material at a rate of 20° C./min. The sample and reference pans were placed under a 60 mL/min and 40 m/min nitrogen purge, respectively. Data analysis was completed using TRIOS (TA Instruments, New Castle, DE). The mass spectrometer was a Discovery MS (TA Instruments, New Castle, DE) benchtop quadrupole instrument.

### Dynamic Vapor Sorption (DVS)

Hygroscopicity was studied using dynamic vapor sorption (DVS, TA Q5000 SA, TA Instruments, New Castle, DE or DVS, DVS Intrinsic, Surface Measurement Systems, London, UK). A sample (2-20 mg) was placed in an aluminum DVS pan and loaded on the sample side of the twin pan balance. The water sorption and desorption were studied as a function of relative humidity (RH) at 25° C. In 10% RH increments, the relative humidity was increased from 5% RH to 95% RH and then decreased back to 5% (some cases from 0 to 90% RH and then decrease back to 0%). Each relative humidity increment had an equilibration time of 120 minutes, unless weight change % was less than 0.01% in 20 minutes. Data analysis was performed using Universal Analysis 2000 Version 4.7A (TA Instruments, New Castle, DE) for TA DVS runs and Microsoft Excel for SMS DVS runs.

### Proton Nuclear Magnetic Resonance (¹H NMR)

Proton Nuclear Magnetic Resonance (¹H NMR) spectra were collected on a Bruker Avance III-HD 400 with SampleXpress. The default proton parameters were spectral width: 16.19 to −3.84 ppm (8012.8 Hz); relaxation delay: 1 sec; pulse: 90 degrees; acquisition time: 4.0894 sec; number of scans or repetitions: 16; temperature: 25° C.

### Amorphous Formula I

Amorphous Formula I was prepared via ball-milling. A cylindrical stainless-steel cell containing a stainless-steel ball was charged with about 5 grams of Form III of Formula I. The cell was capped, and the solids were milled in a Retsch Model MM200 ball mill at a vibration frequency of 30/second for a total time of about 24 minutes. The milling was stopped 3 times during this period to check progress via (Polarized Light Microscopy) PLM, and at the end of the milling PLM showed no signs of birefringence. The material was stored in the cell at about 10° C. The next day the solids were collected and weighed.

Amorphous Formula I was characterized by XRPD, DSC, TGA, and DVS. XRPD was conducted and the diffractogram is depicted in FIG. 1. A DSC thermogram was obtained and is depicted in FIG. 2, which shows a broad endotherm (28-120° C.; 135 J/g; attributed to loss of water), and exothermic events above about 150° C. TGA was performed and the resulting thermogram is depicted in FIG. 3. DVS was performed and the resulting isotherm is shown in FIG. 4.

### Formula I

Formula I was prepared by methods known to persons skilled in the art and/or according to the following reaction scheme.

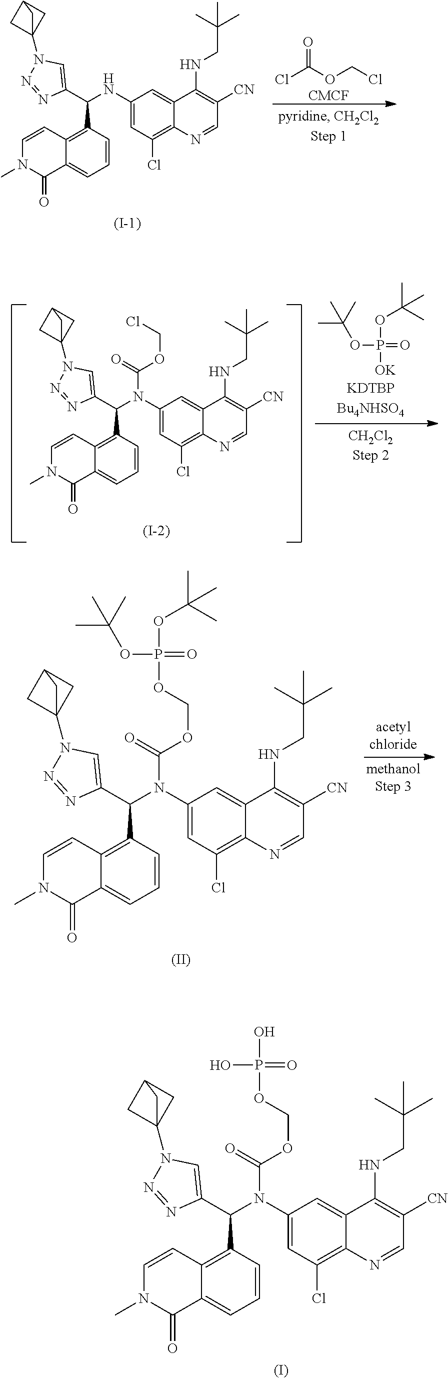

### Installation of Carbamate and Di-Tert-Butylphosphate on (I-1) to Form Formula (II):

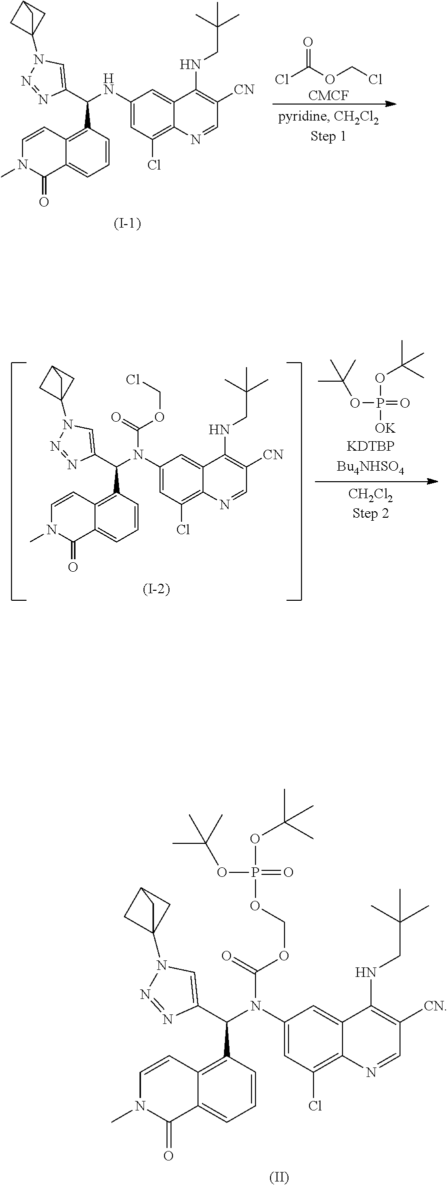

Intermediate (I-1) (1.00 equiv, scaling factor) and dichloromethane (9.8 volumes) were charged to a reactor and the mixture was agitated at about 5° C. Pyridine (1.80 equiv) was charged, followed by chloromethyl chloroformate (1.38 equiv). The mixture was agitated at about 5° C. until the reaction was deemed complete. A 9 wt % aqueous sulfuric acid solution (5.0 volumes) was charged and the mixture was agitated at about 22° C. The layers were separated and the organic layer was washed with a 9 wt % aqueous sulfuric acid solution (5.0 volumes), followed by a 5 wt % aqueous potassium bicarbonate solution (5.0 volumes), affording a solution of Intermediate (I-2) in dichloromethane.

To the reactor containing the Intermediate (I-2)-dichloromethane solution was charged tetra-n-butyl ammonium hydrogen sulfate (0.10 equiv) and potassium di-tert-butylphosphate (1.24 equiv). The mixture was agitated at about 40° C. until the reaction was deemed complete. The mixture was then cooled to about 22° C. and the mixture was washed with water (5.0 volumes). The organic layer was concentrated under vacuum to about 3 volumes and N,N-dimethylacetamide (3.0 volumes) was charged. The mixture was concentrated to about 4 volumes and the temperature was adjusted to about 40° C. Formula (II) seed crystals (0.001 wt equivalents) were charged, the mixture was agitated at about 40° C. for about 1 h, and 2-propanol (12.0 volumes) was charged. The mixture was adjusted to about 10° C., agitated at about 10° C., and the slurry was filtered. The filter cake was washed with 2-propanol (3.0 volumes) and then dried to afford Formula (II). ¹H NMR (400 MHz, CDCl₃): δ 8.54 (s, 1H), 8.29 (d, J=7.9 Hz, 1H), 8.20 (br s, 1H), 7.75 (br s, 1H), 7.54 (s, 1H), 7.51 (d, J=7.3 Hz, 1H), 7.33 (s, 1H), 7.19 (d, J=7.9 Hz, 1H), 7.16 (dd, J=7.6 Hz, 4.6 Hz, 1H), 6.85 (br s, 1H), 6.74 (d, J=7.7 Hz, 1H), 5.65 (dd, J=16.3 Hz, 5.3 Hz, 1H), 5.42 (br d, J=9.2 Hz, 1H), 3.85 (m, 1H), 3.62 (m, 1H), 3.59 (s, 3H), 2.73 (s, 1H), 2.41 (s, 6H), 1.43 (s, 9H), 1.34 (s, 9H0, 1.03 (s, 9H). ¹³C NMR (100 MHz, CDCl₃): δ 162.21, 154.70, 153.18 153.05, 144.88, 143.73, 135.64, 135.04, 133.75, 133.64, 133.43, 132.48, 131.04, 128.59, 126.62, 125.96, 122.02, 119.63, 119.01, 101.21, 84.59, 84.16, 84.09, 83.82, 83.74, 83.62, 83.54, 55.00, 54.77, 53.23, 51.99, 37.14, 33.12, 29.76, 29.72, 29.65, 29.61, 27.21, 23.46.

Deprotection of Formula (II) to form Formula (I)

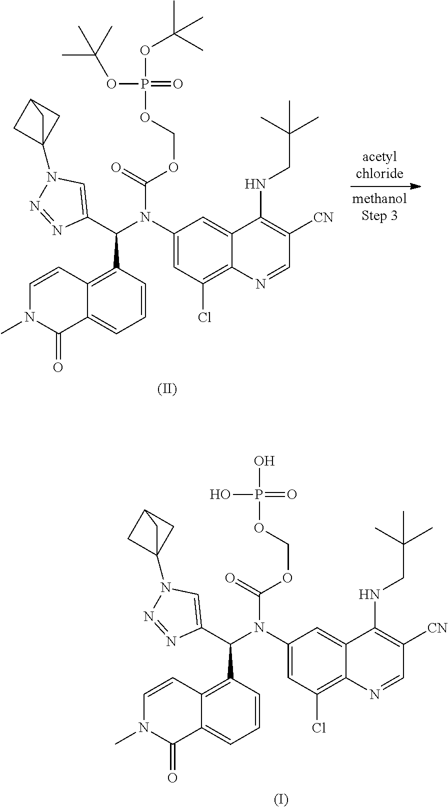 [SMILES: Cn1ccc2c([C@@H](c3cn(C45CC(C4)C5)nn3)N(C(=O)OCOP(=O)(OC(C)(C)C)OC(C)(C)C)c3cc(Cl)c4ncc(C#N)c(NCC(C)(C)C)c4c3)cccc2c1=O ; "(II)" ; Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O ; "(I)"]

Formula (II) (1.00 equiv, scaling factor) and methanol (3.00 volumes) were charged to a reactor. Acetyl chloride (2.97 equiv) was charged while maintaining less than about 30° C. and the mixture was agitated at about 22° C. until the reaction was deemed complete. The mixture was diluted with dichloromethane (5.0 volumes) and 5 wt % aqueous sodium chloride (3.2 volumes) was charged. The layers were separated and the organic layer was then concentrated under vacuum to about 4 volumes. 2-Propanol (5.1 volumes) was charged and the mixture was concentrated under vacuum to about 4.0 volumes. Water (3.0 volumes) was charged and the mixture was agitated at about 22° C. for about 1 hour. Formula (I) seed crystals (0.002 wt equiv) were charged and the mixture was agitated at about 22° C. The slurry was filtered and the filter cake was rinsed with a mixture of water (1.3 volumes) and 2-propanol (1.3 volumes) and then dried to afford Formula (I). ¹H NMR (400 MHz, DMSO-d₆): δ 8.47 (s, 1H), 8.30 (s, 1H), 8.04 (d, J=7.2 Hz, 1H), 7.99 (br s, 1H), 7.96 (s, 1H), 7.92 (br s, 1H), 7.58 (d, J=8.0 Hz, 1H), 7.33 (s, 1H), 7.26 (d, J=6.8 Hz, 1H), 7.19 (t, J=7.6 Hz, 1H), 6.72 (d, J=6.4 Hz, 1H), 3.91 (m, 1H), 5.56 (m, 1H), 5.38 (m, 1H), 3.49 (s, 3H), 3.35 (m, 1H), 2.71 (s, 1H), 2.35 (s, 6H), 0.87 (s, 9H). ¹³C NMR (100 MHz, DMSO-d₆): δ 161.1, 154.5, 152.9, 152.7, 144.8, 142.7, 135.5, 135.0, 134.7, 133.4, 132.7, 131.7, 131.6, 127.5, 125.6, 125.5, 123.4, 119.7, 118.8, 100.1, 83.6, 83.4, 54.3, 53.2, 52.6, 51.5, 36.5, 33.3, 26.8, 22.9.

### Formula I Form I

A reactor was charged with 1 equiv of Formula II and the contents were flushed with nitrogen.

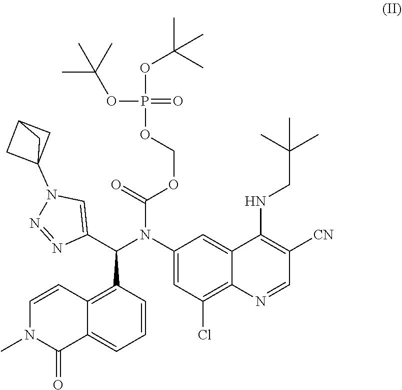 [SMILES: Cn1ccc2c([C@@H](c3cn(C45CC(C4)C5)nn3)N(C(=O)OCOP(=O)(OC(C)(C)C)OC(C)(C)C)c3cc(Cl)c4ncc(C#N)c(NCC(C)(C)C)c4c3)cccc2c1=O]

Methanol (3V) was added, agitation was set to 250 rpm and the reactor internal temperature was adjusted to about 15° C. To the reactor was charged 2.3 equiv of acetyl chloride, and internal temperature was maintained below about 30° C. On completion of the acetyl chloride addition, the internal temperature was maintained at about 20° C. The mixture was agitated for about 2 hours. Dichloromethane (DCM) (about 5 volumes) and 5 wt % aqueous NaCl (about 3 volumes) were added, the mixture was agitated for about 15 minutes, and left at rest overnight. The phases were separated. The organic stream was concentrated to about 3 volumes and diluted with about 5.4 volumes of ethanol, then concentrated again to about 3 volumes. Additional ethanol (about 5.4 volumes) was charged, and the mixture was concentrated again to about 5.4 volumes and diluted with about 0.6 volumes of water. The internal temperature was adjusted to about 15° C. and the mixture was agitated overnight. The slurry was filtered, and the solids were washed with about 1 volume of pre-cooled 9:1 ethanol:water (v:v). The solids were dried at reduced pressure with wet nitrogen at about 25% RH at about 20° C.

Formula I Form I was characterized by XRPD, and the resulting diffractogram is shown in FIG. 5. An XRPD peak list is reported in Table 1.

TABLE 1

| | Position | Relative Intensity |
|---|---|---|
| No. | °2-theta | (%) |
| 1 | 6.2 | 100.0 |
| 2 | 8.3 | 6.5 |
| 3 | 11.6 | 10.3 |
| 4 | 10.9 | 6.7 |
| 5 | 14.5 | 6.0 |
| 6 | 22.4 | 8.1 |
| 7 | 17.4 | 5.9 |
| 8 | 18.7 | 8.4 |
| 9 | 22.6 | 8.7 |

A DSC thermogram was obtained and shows a broad endothermic event from about 25-100° C. and an exothermic event from about 175-225° C. (FIG. 6). TGA-MS analysis was performed and showed mass losses attributed to loss of water of hydration, and evolution of carbon dioxide and formaldehyde due to decomposition. DVS was conducted and the resulting isotherm shows weight change of about 9.5 weight percent from RH 5-95%, and moderate hysteresis.

### Formula I Form II

Method 1: A 4 mL threaded glass vial with stir bar was charged with about 100 mg of Formula I Form I and about 2 mL of 2-propanol/water 1:1. The mixture was capped and stirred at room temperature for about one day, producing Formula I Form II as crystals suspended in solution.

Method 2: A reactor was charged with 1 equiv of Formula II and the contents were flushed with nitrogen. Methanol (about 3 volumes) was added, agitation was set to about 250 rpm and the reactor internal temperature was adjusted to about 15° C. To the reactor was charged 3 equiv of acetyl chloride, and internal temperature was maintained below about 30° C. On completion of acetyl chloride addition, the internal temperature was maintained at about 20° C. The mixture was agitated for about 4 hours. DCM (about 5 volumes) and 5 wt % aqueous NaCl (about 3 volumes) were added, agitated for about 15 minutes, and left at rest overnight. The phases were separated. The organic stream was concentrated to about 4 volumes and diluted with about 5 volumes of 2-propanol, then concentrated to about 4 volumes and diluted with about 3 volumes of water. The internal temperature was adjusted to about 20° C., about 0.005 wt % Formula I Form I seeds were added after about 1 hour and the mixture was agitated overnight. The slurry was filtered, and the solids (Formula I Form II) were washed with about 2.5 volumes of 1:1 (v:v) 2-propanol:water. The solids were dried at reduced pressure at about 20° C.

Formula I Form II was characterized by XRPD, VH-XRD, Single Crystal X-ray Crystallography, and DVS. The resulting XRPD diffractogram is depicted in FIG. 7. An XRPD peak list is shown in Table 2.

TABLE 2

| | Position | Relative Intensity |
|---|---|---|
| No. | °2-theta | (%) |
| 1 | 7.4 | 100.0 |
| 2 | 9.4 | 31.5 |
| 3 | 10.6 | 25.9 |
| 4 | 8.8 | 8.8 |
| 5 | 12.3 | 34.6 |
| 6 | 26.1 | 22.1 |
| 7 | 14.7 | 23.2 |
| 8 | 18.1 | 23.0 |
| 9 | 22.4 | 21.1 |

A diffractogram, obtained at equilibrium at 85% RH in a VH-XRD assay, was indexed using the Pawley method and the unit cell dimensions were determined and reported in Table 3.

TABLE 3

| | | | |
|---|---|---|---|
| Spacegroup | P2₁2₁2₁ | Volume (Å³) | 4008.4 |
| a (Å) | 11.042 | α (°) | 90 |
| b (Å) | 15.045 | β (°) | 90 |
| c (A) | 24.130 | γ (°) | 90 |
| τ_{G} (nm) | 149.4 | ε_{L} | 0.186 |
| SAM (mm) | 13.3 | | |
| R_{wp} | 2.9% | | |

Single Crystal X-ray Crystallography for Form II was conducted, and the data showed Form II is a tetrahydrate.

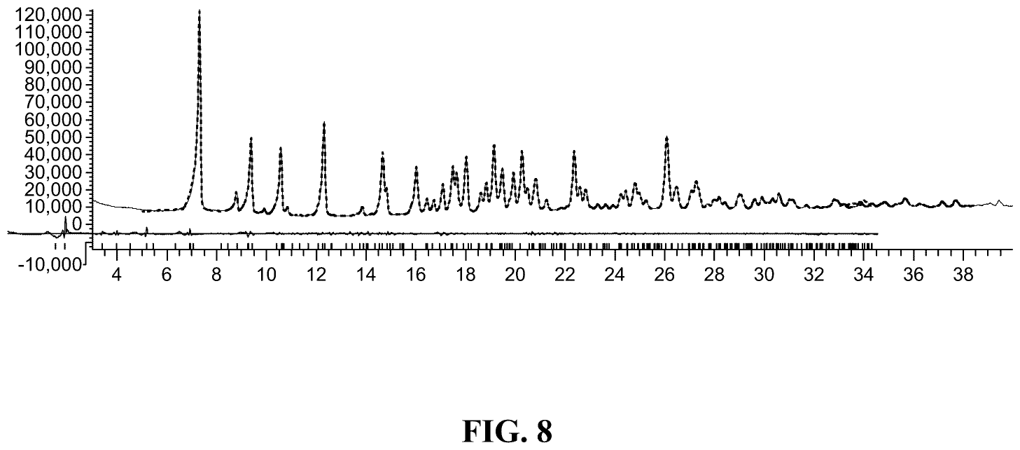

FIG. 8 shows a Whole Pattern Pawley Refinement of Formula I Form II at 85% RH.

DVS showed multiple mass changes as a function of RH, with low hysteresis (FIG. 9). Form II was present in the DVS experiment at the highest RH point.

### Formula I Form III

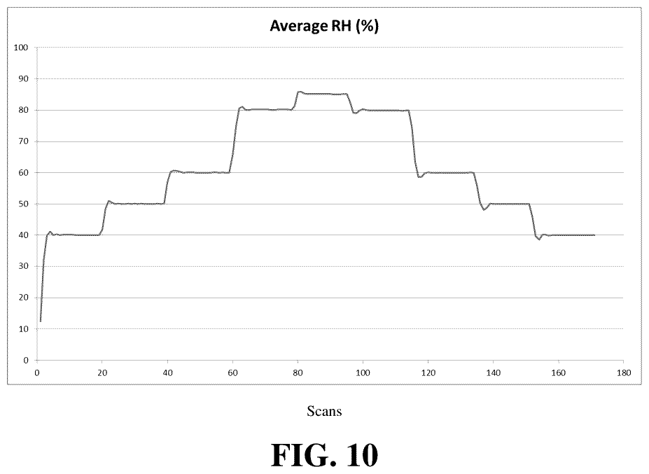

Method 1: Formula I Form III was prepared from Form I and Form II and mixtures thereof by prolonged exposure to RH of 40%. A relative humidity controller was used for the following analysis. The controller was programmed to maintain the following target RH values for 1 hour each 40%, 50%, 60%, 80%, 85%, 80%, 60%, 50%, and 40%. FIG. 10 depicts the measured RH values and the corresponding 5-minute XRPD repeat scans taken during the RH program. The total run time was approximately 14 hours, during which XRPD patterns (5-minute repeats) were collected.

Method 2: A reactor was charged with 1 equiv of Formula II and the contents were flushed with nitrogen. Methanol (about 3 volumes) was added, agitation was set to about 250 rpm and the reactor internal temperature was adjusted to about 15° C. To the reactor was charged about 3 equiv of acetyl chloride, and internal temperature was maintained below about 30° C. After acetyl chloride addition the internal temperature was maintained at about 20° C. The mixture was agitated for about 4 hours. DCM (about 5 volumes) and 5 wt % aqueous NaCl (about 3 volumes) were added, the mixture was agitated for about 15 minutes, and left at rest overnight. The phases were separated. The organic stream was concentrated to about 4 volumes and diluted with about 5 volumes of 2-propanol, then concentrated again to about 4 volumes and diluted with about 3 volumes of water. The internal temperature was adjusted to about 20° C., about 0.002 wt % Form II seeds were added after about 1 hour, and the mixture was agitated overnight. The slurry was filtered, and the solids were washed with about 2.5 volumes of 1:1 (v:v) 2-propanol:water. The solids were dried at reduced pressure at about 20° C.

Method 3: About 1 gram of Formula I Form II was charged to a 20 mL glass vial. The vial was covered with a Kimwipe cloth and left at rest exposed to the ambient atmosphere and temperature (about 47% RH and about 22° C. After about 46 hours temperature and RH were recorded and a sample was taken for XRPD and KF, as is reported below.

Method 4: About 40 mg of Formula I S-Mandelate was charged to a 4 mL glass vial equipped with cap and stir bar. About 0.5 mL of ethanol/water (35:65 v:v) was added, the vial was capped, and the mixture was stirred at room temperature overnight. After stirring overnight solids were isolated and air dried at room temperature overnight and then oven dried at about 40° C. for about 3 hours. XRPD pattern of the oven dried solids was obtained and identified the solid form as Form III, as is reported below.

Samples prepared as described above (Method 1, Method 2, Method 3 and Method 4) were characterized by XRPD, VH-XRD, and DVS. The resulting XRPD is depicted in FIG. 11, which shows XRPD taken at about 36% RH prepared by Method 3. The water content was assessed by KF measurement and found to be 2.3% water (1 mole, a monohydrate form). The 40% RH XRPDs from the VH-XRD experiment (Method 1) are depicted in FIG. 12.

A 40% RH diffractogram was indexed using the Pawley method and the unit cell dimensions were determined. Results can be seen in FIG. 13 and are reported in Table 4.

TABLE 4

| | | | |
|---|---|---|---|
| Spacegroup | P2₁2₁2₁ | Volume (Å³) | 3715.2 |
| a (Å) | 10.993 | α (°) | 90 |
| b (Å) | 14.976 | β (°) | 90 |
| c (Å) | 22.566 | γ (°) | 90 |
| τ_{G} (nm) | 152.4 | ε_{L} | 0.183 |
| SAM (mm) | 14.0 | | |
| R_{wp} | 2.7% | | |

An XRPD peak list for Form III is reported in Table 5.

TABLE 5

| | Position | Relative Intensity |
|---|---|---|
| No. | °2-theta | (%) |
| 1 | 9.8 | 100.0 |
| 2 | 7.8 | 83.8 |
| 3 | 10.7 | 52.3 |
| 4 | 12.5 | 41.0 |
| 5 | 8.9 | 29.2 |
| 6 | 20.1 | 95.7 |
| 7 | 15.5 | 68.2 |
| 8 | 18.2 | 56.0 |
| 9 | 22.9 | 48.3 |

DVS was performed and the resulting isotherm is depicted in FIG. 14.

### Formula I Form IV

Formula I Form IV was prepared from Form III and Form II and mixtures thereof by exposure to dry Nitrogen (0% to 5% RH).

Samples prepared as described above were characterized by VH-XRD, and DVS.

A diffractogram of Formula I Form IV was indexed using the Pawley method (FIG. 15). The unit cell dimensions were determined and are reported in Table 6.

TABLE 6

| | | | |
|---|---|---|---|
| Spacegroup | P2₁2₁2₁ | Volume (Å³) | 3718.3 |
| a (Å) | 11.034 | α (°) | 90 |
| b (Å) | 15.157 | β (°) | 90 |
| c (Å) | 22.232 | γ (°) | 90 |
| τ_{G} (nm) | 167.9 | ε_{L} | 0.264 |
| SAM (mm) | 13.9 | | |
| R_{wp} | 2.5% | | |

An XRPD was collected and the resulting diffractogram is depicted in FIG. 16, which was obtained at equilibrium at 5% RH as described. An XRPD peak list for the 5% RH XRPD is reported in Table 7.

TABLE 7

| | Position | Relative Intensity |
|---|---|---|
| No. | °2-theta | (%) |
| 1 | 20.0 | 100.0 |
| 2 | 8.0 | 19.0 |
| 3 | 18.1 | 76.6 |
| 4 | 9.0 | 33.2 |
| 5 | 9.9 | 65.8 |
| 6 | 10.8 | 47.7 |
| 7 | 15.6 | 57.3 |
| 8 | 22.8 | 47.3 |
| 9 | 24.9 | 34.1 |

### Formula I Form V

A 20 mL threaded glass vessel was charged with about 1.4 g Formula I Form I and about 10 mL of ethanol/water solution (9:1 v:v). The mixture formed a suspension, the vial was capped, and the suspension was agitated on a nutating mixer at room temperature. After 12 days a sample was acquired for testing via centrifuge-filtration.

Formula I Form V was characterized by XRPD, DSC, and TGA-MS. The resulting XRPD is shown in FIG. 17. An XRPD peak list is reported in Table 8.

TABLE 8

| | Position | Relative Intensity |
|---|---|---|
| No. | °2-theta | (%) |
| 1 | 6.23 | 100.0 |
| 2 | 22.6 | 45.9 |
| 3 | 20.4 | 36.6 |
| 4 | 16.3 | 16.9 |
| 5 | 16.5 | 21.3 |
| 6 | 17.4 | 21.2 |
| 7 | 22.4 | 30.8 |
| 8 | 23.5 | 25.1 |
| 9 | 25.1 | 22.0 |

A DSC thermogram was obtained and is depicted in FIG. 18. TGA-MS was performed, and the resulting data showed a combined loss of 7.3 weight percent water and ethanol from about 50 to 150° C., and loss of formaldehyde and carbon dioxide due to decomposition above 150° C.

### Formula I Form VI

A 20 mL threaded glass vessel was charged with about 1.4 g Formula I Form I and about 10 mL of ethanol/water solution (7.4:2.6). The mixture formed a suspension, the vial was capped, and the suspension was agitated on a nutating mixer at room temperature. After 12 days a sample was acquired for testing via centrifuge-filtration.

Formula I Form VI was characterized by XRPD, DSC, and TGA-MS. The resulting XRPD diffractogram is depicted in FIG. 19. A second image with the vertical axis limit set to 2000 counts is included (FIG. 20). An XRPD peak list is reported in Table 9.

TABLE 9

| | Position | Relative Intensity |
|---|---|---|
| No. | °2-theta | (%) |
| 1 | 7.2 | 100.0 |
| 2 | 14.4 | 9.6 |
| 3 | 21.7 | 4.2 |
| 4 | 29.1 | 2.9 |
| 5 | 25.3 | 1.3 |
| 6 | 25.0 | 0.7 |
| 7 | 16.6 | 0.23 |
| 8 | 26.7 | 0.28 |
| 9 | 30.2 | 0.13 |

A DSC thermogram was obtained and is depicted in FIG. 21. TGA-MS was performed and showed loss of water of about 3.8 wt % at 30° C. during a drying period of 15 minutes, and a combined mass loss of water and ethanol of about 7.4 wt % from about 30-200° C. This second mass loss showed ions for water and ethanol being detected at different times. Additional mass loss detected above about 150° C. was attributed to decomposition due to loss of carbon dioxide, water, and formaldehyde.

### Formula I Form VII

Formula I Form VII was prepared by stirring a mixture of about 0.3 grams of Formula I Form I in about 3 mL of methanol at room temperature for several days. Solids were isolated by centrifuge-filtration and characterized.

Formula I Form VII was characterized by XRPD and TGA-MS. The resulting XRPD diffractogram is depicted in FIG. 22. An XRPD peak list is reported in Table 10.

TABLE 10

| | Position | Relative Intensity |
|---|---|---|
| No. | °2-theta | (%) |
| 1 | 6.2 | 100.0 |
| 2 | 8.4 | 10.4 |
| 3 | 22.6 | 31.5 |
| 4 | 10.3 | 8.7 |
| 5 | 10.9 | 9.3 |
| 6 | 11.7 | 11.0 |
| 7 | 16.1 | 12.4 |
| 8 | 16.4 | 15.6 |
| 9 | 17.3 | 13.7 |

TGA-MS was performed and the resulting thermogram showed mass loss of about 2.9 wt % mass, identified as methanol and water, from about 50 to 125° C. Mass loss above about 150° C. was attributed to carbon dioxide, water, and formaldehyde, due to decomposition.

### Formula I Form VIII

Formula I Form VIII was prepared by stirring a mixture of about 0.2-0.4 grams of Formula I Form I in about 3 mL of ethanol at room temperature for several days. Solids were isolated by centrifuge-filtration and characterized.

Formula I Form VIII was characterized by XRPD and TGA-MS. The resulting XRPD diffractogram is shown in FIG. 23. An XRPD peak list is reported in Table 11.

TABLE 11

| | Position | Relative Intensity |
|---|---|---|
| No. | °2-theta | (%) |
| 1 | 6.2 | 100.0 |
| 2 | 8.3 | 8.3 |
| 3 | 22.3 | 20.3 |
| 4 | 11.6 | 9.56 |
| 5 | 10.8 | 8.0 |
| 6 | 14.5 | 10.9 |
| 7 | 16.5 | 8.0 |
| 8 | 18.7 | 8.4 |
| 9 | 20.5 | 8.7 |

TGA-MS was performed and the resulting thermogram showed combined mass loss of about 5.9 wt %, identified as ethanol and water, from about 50 to 125° C. Ethanol and water peaks overlapped. Mass loss above 156° C. was attributed to decomposition products carbon dioxide, water, and formaldehyde.

### Formula I Form IX

Formula I Form IX was prepared by stirring a mixture of about 0.2-0.4 grams of Formula I Form I in about 3 mL of acetone at room temperature for several days. Solids were isolated by centrifuge-filtration and characterized.

Formula I Form IX was characterized by XRPD and TGA-MS. The resulting XRPD diffractogram is depicted in FIG. 33. An XRPD peak list is reported in Table 12.

TABLE 12

| | Position | Relative Intensity |
|---|---|---|
| No. | °2-theta | (%) |
| 1 | 7.2 | 100.0 |
| 2 | 5.8 | 69.4 |
| 3 | 5.7 | 28.5 |
| 4 | 10.7 | 8.8 |
| 5 | 15.3 | 3.9 |
| 6 | 17.1 | 6.0 |
| 7 | 7.4 | 8.8 |
| 8 | 10.0 | 4.1 |
| 9 | 8.9 | 3.6 |

TGA-MS was performed and showed overlapping mass loss of about 6.3% of surface water and acetone during a 30° C. drying step and overlapping mass loss of about 1.8% from about 30 to 150° C., also attributed to water and acetone. Mass loss above 150° C. was observed and attributed to decomposition products carbon dioxide, water, and formaldehyde.

### Formula I Form X

Formula I Form X was prepared by stirring a mixture of about 0.2-0.4 grams of Formula I Form I in about 3 mL of THF at room temperature for several days. Solids were isolated by centrifuge-filtration and characterized.

Formula I Form X was characterized by XRPD and TGA-MS. The resulting XRPD diffractogram is shown in FIG. 25. An XRPD peak list is reported in Table 13.

TABLE 13

| | Position | Relative Intensity |
|---|---|---|
| No. | °2-theta | (%) |
| 1 | 16.3 | 100 |
| 2 | 7.2 | 94.7 |
| 3 | 5.7 | 85.6 |
| 4 | 10.8 | 36.6 |
| 5 | 13.7 | 17.6 |
| 6 | 18.3 | 24.0 |
| 7 | 19.1 | 24.0 |
| 8 | 22.4 | 19.6 |
| 9 | 26.5 | 10.3 |

TGA-MS was performed and the resulting thermogram showed overlapping mass loss of about 8.5% of surface water and THF during a 30° C. drying step and overlapping mass loss of about 8.5% from about 30 to 150° C., also attributed to water and THF. Mass loss above 150° C. was attributed to decomposition products carbon dioxide, water, and formaldehyde.

### Formula I Form XI

Formula I Form XI was prepared by stirring a mixture of about 0.2-0.4 grams of Formula I Form I in about 3 mL of DCM at room temperature for several days. Solids were isolated by centrifuge-filtration and characterized.

Formula I Form XI was characterized by XRPD and TGA-MS. The resulting XRPD diffractogram is shown in FIG. 26. An XRPD peak list is reported in Table 14.

TABLE 14

| | Position | Relative Intensity |
|---|---|---|
| No. | °2-theta | (%) |
| 1 | 7.8 | 100.0 |
| 2 | 5.5 | 41.5 |
| 3 | 6.8 | 27.4 |
| 4 | 8.6 | 51.0 |
| 5 | 9.5 | 16.2 |
| 6 | 10.3 | 28.3 |
| 7 | 15.4 | 13.2 |
| 8 | 17.8 | 30.5 |
| 9 | 19.4 | 22.0 |

TGA-MS was performed and showed mass loss of about 2.6% of surface water during a 30° C. drying step and overlapping mass loss of about 3.8% from about 30 to 150° C., attributed to water and DCM. Mass loss above 150° C. was observed and attributed to decomposition products carbon dioxide, water, and formaldehyde.

### Formula I Form XII

Formula I Form XII was prepared by stirring a mixture of about 0.2-0.4 grams of Formula I Form I in about 3 mL of MTBE at room temperature for several days. Solids were isolated by centrifuge-filtration and characterized.

Formula I Form XII was characterized by XRPD and TGA-MS. The resulting XRPD diffractogram is shown in FIG. 27. An XRPD peak list is reported in Table 15.

TABLE 15

| | Position | Relative Intensity |
|---|---|---|
| No. | °2-theta | (%) |
| 1 | 6.2 | 100.0 |
| 2 | 5.4 | 14.3 |
| 3 | 11.5 | 9.6 |
| 4 | 14.5 | 7.5 |
| 5 | 16.4 | 10.6 |
| 6 | 22.3 | 9.0 |
| 7 | 12.5 | 4.6 |
| 8 | 9.7 | 2.4 |
| 9 | 19.2 | 4.2 |

TGA-MS was performed and showed mass loss of about 4.2% of surface water during a 30° C. drying step and overlapping mass loss of about 1.2% from about 30 to 150° C., attributed to water. Loss of MTBE was seen from about 100-200° C. and overlapped mass loss attributed to decomposition products carbon dioxide, water, and formaldehyde.

### Formula I Form XIII

Formula I Form XIII was prepared by stirring a mixture of about 0.2-0.4 grams of Formula I Form I in about 3 mL of IPA at room temperature for several days. Solids were isolated by centrifuge-filtration and characterized.

Formula I Form XIII was characterized by XRPD and TGA-MS. The resulting XRPD diffractogram is shown in FIG. 28. An XRPD peak list is reported in Table 16.

TABLE 16

| | Position | Relative Intensity |
|---|---|---|
| No. | °2-theta | (%) |
| 1 | 5.8 | 100 |
| 2 | 6.2 | 32.8 |
| 3 | 8.1 | 9.4 |
| 4 | 11.6 | 5.7 |
| 5 | 16.6 | 9.9 |
| 6 | 20.0 | 8.8 |
| 7 | 13.0 | 5.0 |
| 8 | 22.0 | 5.9 |
| 9 | 22.8 | 13.2 |

TGA-MS was performed and showed mass loss of about 3.7% of surface water and IPA during a 30° C. drying step and overlapping mass loss of about 10.7% from about 30 to 150° C., attributed to water and IPA. Mass loss above 150° C. was attributed to decomposition products carbon dioxide, water, and formaldehyde.

### Formula I Form XIV

Formula I Form XIV was prepared by stirring a mixture of about 0.2-0.4 grams of Formula I Form I in about 3 mL of 1-propanol at room temperature for several days. Solids were isolated by centrifuge-filtration and characterized.

Formula I Form XIX was characterized by XRPD and TGA-MS. The resulting XRPD diffractogram is shown in FIG. 29. An XRPD peak list is reported in Table 17.

TABLE 17

| | Position | Relative Intensity |
|---|---|---|
| No. | °2-theta | (%) |
| 1 | 5.8 | 100 |
| 2 | 8.2 | 12.0 |
| 3 | 18.5 | 23.0 |
| 4 | 11.7 | 7.7 |
| 5 | 16.6 | 10.9 |
| 6 | 22.0 | 13.7 |
| 7 | 22.8 | 23.9 |
| 8 | 10.0 | 4.9 |
| 9 | 10.5 | 4.1 |

TGA-MS was performed and showed mass loss of about 1.6% of surface water and 1-propanol during a 30° C. drying step and overlapping mass loss of about 11.6% from about 30 to 150° C., attributed to water and 1-propanol. Mass loss above 150° C. was observed and attributed to decomposition products carbon dioxide, water, and formaldehyde.

### Formula I Form XV

Formula I Form XV was prepared by stirring a mixture of about 0.2-0.4 grams of Formula I Form I in about 3 mL of CPME at room temperature for several days. Solids were isolated by centrifuge-filtration and characterized.

Formula I Form XV was characterized by XRPD and TGA-MS. The resulting XRPD diffractogram is shown in FIG. 30. An XRPD peak list is reported in Table 18.

TABLE 18

| | Position | Relative Intensity |
|---|---|---|
| No. | °2-theta | (%) |
| 1 | 6.2 | 100 |
| 2 | 5.4 | 7.8 |
| 3 | 8.3 | 8.2 |
| 4 | 11.6 | 19.4 |
| 5 | 16.4 | 22.1 |
| 6 | 19.3 | 20.2 |
| 7 | 12.4 | 15.5 |
| 8 | 20.3 | 18.2 |
| 9 | 22.4 | 17.8 |

TGA-MS was performed and showed mass loss of about 8.3% of surface water and CPME during a 30° C. drying step and overlapping mass loss of about 3.2% from about 30 to 150° C., attributed to water and CPME. CPME was observed from about 5-200° C., and overlapped mass loss attributed to decomposition products carbon dioxide, water, and formaldehyde.

Formula I 2-(4-Hydroxybenzoyl) benzoate

A 4 mL threaded glass vial was charged with about 0.2 g of Formula I, about 2 eq. 2-(4-Hydroxybenzoyl) benzoic acid, and about 3 mL of isopropyl acetate. The vial was capped and agitated at room temperature for about 19 days. Solids were isolated by centrifugation.

Formula I 2-(4-Hydroxybenzoyl) benzoate was characterized by XRPD, DSC, TGA, and ¹H NMR. The resulting XRPD diffractogram of freshly isolated solids as wet cake (Formula I 2-(4-Hydroxybenzoyl) benzoate Form A) is shown in FIG. 31. An XRPD peak list is reported in Table 19.

TABLE 19

| | Position | Relative Intensity |
|---|---|---|
| No. | °2-theta | (%) |
| 1 | 6.2 | 100 |
| 2 | 11.0 | 7.5 |
| 3 | 13.7 | 7.6 |
| 4 | 15.2 | 6.6 |
| 5 | 17.4 | 5.3 |
| 6 | 18.3 | 9.6 |
| 7 | 20.8 | 6.5 |
| 8 | 7.6 | 3.9 |
| 9 | 8.5 | 4.4 |

Formula I 2-(4-Hydroxybenzoyl) benzoate Form A, described above, was air-dried. The resulting solid form, Formula I 2-(4-Hydroxybenzoyl) benzoate Form B, was characterized. The XRPD diffractogram of Formula I 2-(4-Hydroxybenzoyl) benzoate Form B is depicted in FIG. 32. The DSC thermogram of Formula I 2-(4-Hydroxybenzoyl) benzoate Form B was conducted and the resulting thermogram is shown in FIG. 33, which shows a broad endothermic event from about 30-100° C. and broad endothermic and exothermic events above about 150° C. TGA of Formula I 2-(4-Hydroxybenzoyl) benzoate Form A was performed and the resulting thermogram is depicted in FIG. 34. ¹H NMR showed about 0.63 wt % IPAc (about 0.07 moles) and Formula I: 2-(4-Hydroxybenzoyl) benzoate ratio of 1.0:0.96.

### Formula I Vanillate

A 4 mL threaded glass vial was charged with about 0.2 g of Formula I, about 2 eq. of Vanillic acid, and about 3 mL of isopropyl acetate. The vial was capped and agitated at room temperature for about 19 days. Solids were isolated by centrifugation for testing.

Formula I Vanillate was characterized by XRPD, DSC, TGA, and ¹H NMR. The resulting XRPD diffractogram of freshly isolated solids (Formula I Vanillate Form A) is shown in FIG. 35. An XRPD peak list for Formula I Vanillate Form A is reported in Table 20.

TABLE 20

| | Position | Relative Intensity |
|---|---|---|
| No. | °2-theta | (%) |
| 1 | 6.2 | 7.8 |
| 2 | 7.3 | 100.0 |
| 3 | 16.4 | 13.8 |
| 4 | 17.5 | 6.3 |
| 5 | 24.7 | 6.2 |
| 6 | 30.6 | 6.7 |
| 7 | 12.4 | 4.8 |
| 8 | 13.4 | 3.9 |
| 9 | 20.2 | 4.3 |

Formula I Vanillate Form A, described above, was air-dried. The resulting solid form, Formula I Vanillate Form B, was characterized. The XRPD diffractogram of Formula I Vanillate Form B is depicted in FIG. 36. DSC thermogram of Formula I Vanillate Form B was conducted and the resulting thermogram is shown in FIG. 37, which shows a broad endothermic event from about 30-100° C. and broad endothermic and exothermic events above about 150° C. TGA of Formula I Vanillate Form B was performed and the resulting thermogram is depicted in FIG. 38. ¹H NMR showed about 1.5 wt % IPAc and a Formula I: Vanillic acid ratio of 1:0.44.

### Formula I Hippurate

A 4 mL threaded glass vial was charged with about 0.2 g of Formula I, about 2 eq. of Hippuric acid, and about 3 mL of isopropyl acetate. The vial was capped and agitated at room temperature for about 19 days. Solids were isolated by centrifugation for testing.

Formula I Hippurate was characterized by XRPD, DSC, TGA, and ¹H NMR. The resulting XRPD diffractogram of freshly isolated solids (Formula I Hippurate Form A) is shown in FIG. 39. An XRPD peak list for Formula I Hippurate Form A is reported in Table 21.

TABLE 21

| | Position | Relative Intensity |
|---|---|---|
| No. | °2-theta | (%) |
| 1 | 6.5 | 100.0 |
| 2 | 8.2 | 48.1 |
| 3 | 9.3 | 41.5 |
| 4 | 7.0 | 35.9 |
| 5 | 13.1 | 63.6 |
| 6 | 21.8 | 89.9 |
| 7 | 14.7 | 15.4 |
| 8 | 18.0 | 28.6 |
| 9 | 25.7 | 23.6 |

Formula I Hippurate Form A, described above, was air-dried. The resulting solid form, Formula I Hippurate Form B, was characterized. The XRPD diffractogram of Formula I Hippurate Form B is depicted in FIG. 40. DSC thermogram of Formula I Hippurate Form B was conducted and the resulting thermogram is shown in FIG. 41, which shows a broad endothermic event from about 30-100° C. and broad endothermic and exothermic events above about 150° C. TGA of Formula I Hippurate Form B was performed and the resulting thermogram is depicted in FIG. 42. ¹H NMR showed about 0.45 wt % IPAc and a Formula I: Hippuric acid ratio of 1:0.49.

### Formula I Maleate

A 4 mL threaded glass vial was charged with about 0.2 g of Formula I, about 2 eq. of Maleic acid, and about 2 mL of isopropyl acetate. The vial was capped and agitated at room temperature for about 12 days. Solids were isolated by centrifugation for testing.

Formula I Maleate was characterized by XRPD, DSC, TGA, and ¹H NMR. The resulting XRPD diffractogram of freshly isolated solids (Formula I Maleate Form A) is shown in FIG. 43. An XRPD peak list is reported in Table 22.

TABLE 22

| | Position | Relative Intensity |
|---|---|---|
| No. | °2-theta | (%) |
| 1 | 5.8 | 100.0 |
| 2 | 8.2 | 10.0 |
| 3 | 11.7 | 11.3 |
| 4 | 10.0 | 8.5 |
| 5 | 10.4 | 10.3 |
| 6 | 14.9 | 9.8 |
| 7 | 6.4 | 5.9 |
| 8 | 20.0 | 9.9 |
| 9 | 25.7 | 6.7 |

Formula I Maleate Form A, described above, was air-dried. The resulting solid form, Formula I Maleate Form B, was characterized. The XRPD diffractogram of Formula I Maleate Form B is shown in FIG. 44. An XRPD peak list is included in Table 23.

TABLE 23

| | Position | Relative Intensity |
|---|---|---|
| No. | °2-theta | (%) |
| 1 | 6.1 | 100.0 |
| 2 | 8.3 | 8.6 |
| 3 | 10.7 | 11.6 |
| 4 | 14.4 | 19.4 |
| 5 | 16.4 | 31.6 |
| 6 | 20.1 | 22.1 |
| 7 | 21.4 | 14.9 |
| 8 | 22.7 | 29.0 |
| 9 | 28.4 | 15.6 |

DSC thermogram of Formula I Maleate Form B was conducted and the resulting thermogram is depicted in FIG. 45, which shows a broad endothermic event from about 30-100° C. and broad endothermic and exothermic events above about 150° C. TGA thermogram of Formula I Maleate Form B was performed and the resulting thermogram is shown in FIG. 46. ¹H NMR showed a Formula I: Maleic acid ratio of about 1:0.4 and about 0.05 wt % IPAc.

### Formula I Glyoxylate

A 4 mL threaded glass vial was charged with about 0.1 g of Formula I, about 2 eq. of glyoxylic acid, and about 2 mL of isopropyl acetate. The vial was capped and agitated at room temperature for about 12 days. Solids were isolated by centrifugation for testing.

Formula I glyoxylate was characterized by XRPD, DSC, TGA, and ¹H NMR. The resulting XRPD diffractogram of freshly isolated solids (Formula I Glyoxylate Form A) is shown in FIG. 47. An XRPD peak list is reported in Table 24.

TABLE 24

| | Position | Relative Intensity |
|---|---|---|
| No. | °2-theta | (%) |
| 1 | 5.9 | 100.0 |
| 2 | 22.5 | 58.1 |
| 3 | 8.1 | 13.2 |
| 4 | 12.4 | 19.0 |
| 5 | 16.4 | 37.2 |
| 6 | 19.1 | 37.3 |
| 7 | 15.0 | 18.0 |
| 8 | 20.0 | 32.4 |
| 9 | 28.1 | 24.8 |

Formula I Glyoxylate Form A, described above, was air-dried. The resulting solid form, Formula I Glyoxylate Form B, was characterized. The XRPD diffractogram of Formula I Glyoxylate Form B is shown FIG. 48. An XRPD peak list is reported in Table 25.

TABLE 25

| | Position | Relative Intensity |
|---|---|---|
| No. | °2-theta | (%) |
| 1 | 6.2 | 100 |
| 2 | 8.3 | 14.4 |
| 3 | 12.6 | 13.9 |
| 4 | 10.8 | 9.0 |
| 5 | 16.1 | 12.9 |
| 6 | 16.4 | 16.4 |
| 7 | 18.6 | 19.4 |
| 8 | 19.4 | 19.3 |
| 9 | 20.3 | 22.9 |

DSC thermogram of Formula I Glyoxylate Form B was conducted and the resulting thermogram is depicted in FIG. 49. TGA of Formula I Glyoxylate Form B was performed and the resulting thermogram is depicted in FIG. 50. ¹H NMR of the solids showed the Formula I: Glyoxylic ratio is about 1.0:0.77, and about 3.0 wt % IPAc is present.

### Formula I L-Pyroglutamate

A 4 mL threaded glass vial was charged with about 0.1 g of Formula I, about 2 eq. of L-Pyroglutamic acid, and about 2 mL of isopropyl acetate. The vial was capped and agitated at room temperature for about 12 days. Solids were isolated by centrifugation for testing.

Formula I L-Pyroglutamate was characterized by XRPD, DSC, and ¹H NMR. The resulting XRPD diffractogram of (wet cake) is shown FIG. 67. An XRPD peak list is reported in Table 26.

TABLE 26

| | Position | Relative Intensity |
|---|---|---|
| No. | °2-theta | (%) |
| 1 | 6.1 | 88.4 |
| 2 | 16.5 | 24.6 |
| 3 | 19.8 | 100.0 |
| 4 | 11.6 | 13.3 |
| 5 | 12.4 | 13.8 |
| 6 | 17.3 | 13.8 |
| 7 | 22.8 | 34.4 |
| 8 | 23.0 | 39.8 |
| 9 | 28.4 | 11.5 |

Formula I L-Pyroglutamate was air-dried and characterized by DSC (FIG. 52). ¹H NMR of the solids showed the Formula I: L-Pyroglutamic ratio was about 1.0:0.63, and about 0.8 wt % IPAc was present.

### Formula I2-Naphthalene Sulfonate

A 4 mL threaded glass vial was charged with about 0.1 g of Formula I, about 1 eq. of 2-Naphthalene sulfonic acid, and about 3 mL of isopropyl acetate. The vial was capped and agitated at room temperature for about 12 days. Solids were isolated by centrifugation

Formula I 2-Naphthalene sulfonate was characterized by XRPD, DSC, TGA, and ¹H NMR. The resulting XRPD diffractogram of freshly prepared solids (wet cake) is shown in FIG. 53. An XRPD peak list is reported in Table 27.

TABLE 27

| | Position | Relative Intensity |
|---|---|---|
| No. | °2-theta | (%) |
| 1 | 5.9 | 100.0 |
| 2 | 8.1 | 15.9 |
| 3 | 16.3 | 38.5 |
| 4 | 18.6 | 23.8 |
| 5 | 19.1 | 28.2 |
| 6 | 20.0 | 26.1 |
| 7 | 10.1 | 9.1 |
| 8 | 12.6 | 10.6 |
| 9 | 13.1 | 8.4 |

Formula I 2-Naphthalene Sulfonate was air-dried and subjected to XRPD. The resultant XRPD diffractogram of Formula I 2-Naphthalene Sulfonate is shown in FIG. 54. An XRPD peak list is reported in Table 28.

TABLE 28

| | Position | Relative Intensity |
|---|---|---|
| No. | °2-theta | (%) |
| 1 | 6.2 | 100.0 |
| 2 | 8.4 | 8.6 |
| 3 | 16.5 | 17.5 |
| 4 | 10.8 | 7.3 |
| 5 | 11.7 | 13.4 |
| 6 | 19.1 | 14.9 |
| 7 | 12.6 | 4.7 |
| 8 | 14.6 | 6.1 |
| 9 | 22.6 | 13.8 |

DSC thermogram of air-dried Formula I 2-Naphthalene Sulfonate was conducted and the resulting thermogram is depicted in FIG. 55. TGA was performed on air-dried solids and the thermogram of Formula I 2-Naphthalene Sulfonate is depicted in FIG. 56. ¹H NMR of the air-dried solids showed the Formula I: 2-Naphthalene sulfonate ratio was 1.0:0.99, and 3.1 wt % IPAc was present.

### Formula I 1-Naphthalene Sulfonate

A 4 mL threaded glass vial was charged with about 0.1 g of Formula I, about 1 eq. of 1-Naphthalene sulfonic acid, and about 3 mL of isopropyl acetate. The vial was capped and agitated at room temperature for about 12 days. Solids were isolated by centrifugation for testing.

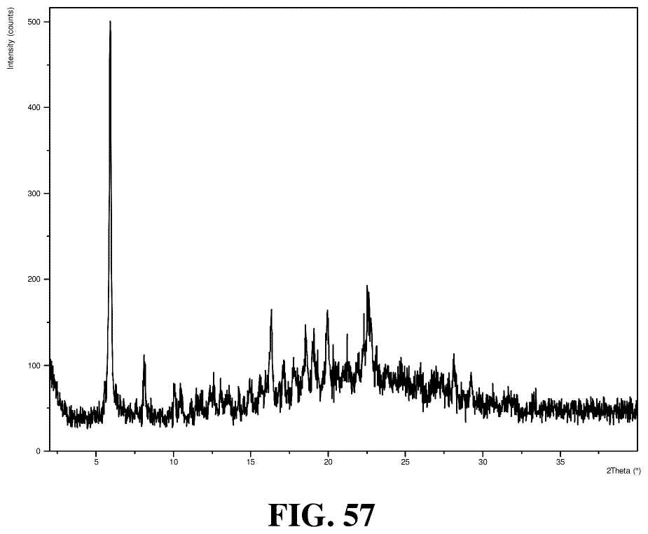

The resultant Formula I 1-Naphthalene Sulfonate was characterized by XRPD, DSC, and ¹H NMR. The resulting XRPD diffractogram of freshly prepared solids (Formula I 1-Naphthalene wet cake) is shown in FIG. 57. An XRPD peak list is reported in Table 29.

TABLE 29

| | Position | Relative Intensity |
|---|---|---|
| No. | °2-theta | (%) |
| 1 | 5.9 | 100.0 |
| 2 | 8.1 | 11.7 |
| 3 | 16.3 | 21.0 |
| 4 | 10.0 | 7.6 |
| 5 | 10.5 | 6.1 |
| 6 | 20.0 | 17.9 |
| 7 | 18.6 | 13.2 |
| 8 | 19.0 | 9.3 |
| 9 | 22.6 | 17.9 |

Formula I 1-Naphthalene Sulfonate wet cake, described above, was air-dried and subjected to XRPD analysis. The XRPD diffractogram of air-dried Formula I 1-Naphthalene Sulfonate is depicted in FIG. 58. An XRPD peak list is reported in Table 30.

TABLE 30

| | Position | Relative Intensity |
|---|---|---|
| No. | °2-theta | (%) |
| 1 | 6.2 | 100.0 |
| 2 | 8.4 | 8.8 |
| 3 | 16.5 | 18.0 |
| 4 | 10.9 | 8.9 |
| 5 | 11.7 | 14.3 |
| 6 | 20.5 | 12.5 |
| 7 | 14.6 | 7.8 |
| 8 | 15.3 | 8.3 |
| 9 | 17.3 | 9.5 |

DSC of air-dried Formula I 1-Naphthalene Sulfonate was performed and the resulting thermogram is shown in FIG. 59. ¹H NMR of the solids showed the Formula I: 1-NSA ratio was 1.0:0.60, and 4.0 wt % IPAc was present.

### Formula I 1-Hydroxy-2-Naphthoate

A 4 mL threaded glass vial was charged with about 0.1 g of Formula I, about 2 eq. of 1-hydroxy-2-naphthoic acid, and about 3 mL of isopropyl acetate. The vial was capped and agitated at room temperature for about 12 days. Solids were isolated by centrifugation for testing.

Formula I 1-Hydroxy-2-Naphthoate was characterized by XRPD, DSC, TGA, and ¹H NMR. The resulting XRPD diffractogram of freshly prepared solids (Formula I 1-Hydroxy-2-Naphthoate wet cake) is shown in FIG. 60. An XRPD peak list is reported in Table 31.

TABLE 31

| | Position | Relative Intensity |
|---|---|---|
| No. | °2-theta | (%) |
| 1 | 5.3 | 75.0 |
| 2 | 5.6 | 100.0 |
| 3 | 7.4 | 62.1 |
| 4 | 6.7 | 57.3 |
| 5 | 9.3 | 46.8 |
| 6 | 21.0 | 35.8 |
| 7 | 17.5 | 21.3 |
| 8 | 18.5 | 27.1 |
| 9 | 23.6 | 30.6 |

Formula I 1-Hydroxy-2-Naphthoate was air-dried and subjected to XRPD. The XRPD diffractogram of air-dried Formula I 1-Hydroxy-2-Naphthoate is shown in FIG. 61. An XRPD peak list is reported in Table 32.

TABLE 32

| | Position | Relative Intensity |
|---|---|---|
| No. | °2-theta | (%) |
| 1 | 6.1 | 100.0 |
| 2 | 7.3 | 47.0 |
| 3 | 15.9 | 18.0 |

DSC of air-dried Formula I 1-Hydroxy-2-Naphthoate was performed and the resulting thermogram is depicted in FIG. 62. A TGA thermogram of Formula I 1-Hydroxy-2-Naphthoate is shown in FIG. 63. ¹H NMR of Form B showed the Formula I: 1-hydroxy-2-naphthoic acid ratio was about 1.0:0.43, and about 1.4 wt % IPAc was present.

### Formula I S-Mandelate

A 4 mL threaded glass vial was charged with about 0.2 g of Formula I, about 2 eq. of S-Mandelic acid, and about 3 mL of isopropyl acetate. The vial was capped and agitated at room temperature for about 30 days. Solids were isolated by centrifugation.

Formula I S-Mandelate was characterized by XRPD, DSC, TGA, and ¹H NMR. The resulting XRPD diffractogram of freshly prepared solids (wet cake) (Formula I S-Mandelate Form A) is depicted in FIG. 64. An XRPD peak list is reported in Table 33.

TABLE 33

| | Position | Relative Intensity |
|---|---|---|
| No. | °2-theta | (%) |
| 1 | 5.5 | 100.0 |
| 2 | 5.7 | 97.0 |
| 3 | 6.3 | 26.0 |
| 4 | 11.4 | 30.4 |
| 5 | 16.6 | 36.5 |
| 6 | 20.8 | 24.9 |
| 7 | 10.0 | 10.7 |
| 8 | 10.4 | 10.5 |
| 9 | 19.0 | 22.6 |

Formula I S-Mandelate Form A, described above, was air-dried. The resulting solid form, Formula I S-Mandelate Form B, was subjected to XRPD analysis. The XRPD diffractogram of Formula I S-Mandelate Form B is shown in FIG. 65. An XRPD peak list of the Formula I S-Mandelate Form B is reported in Table 34.

TABLE 34

| | Position | Relative Intensity |
|---|---|---|
| No. | °2-theta | (%) |
| 1 | 5.7 | 100.0 |
| 2 | 6.2 | 76.5 |
| 3 | 22.6 | 90.2 |
| 4 | 8.0 | 15.8 |
| 5 | 8.4 | 8.9 |
| 6 | 11.7 | 58.7 |
| 7 | 13.5 | 24.4 |
| 8 | 16.0 | 39.3 |
| 9 | 16.6 | 56.6 |

DSC thermogram of Formula I S-Mandelate Form B was conducted and the resulting thermogram is shown in FIG. 66 and shows a broad endothermic event from about 50 to 75° C., a sharp endothermic event at about 150° C. Endothermic and exothermic events at higher temperatures were attributed to decomposition. TGA thermogram of Formula I S-Mandelate Form B is depicted in FIG. 67 and shows a mass loss of about 3.0 weight percent up to about 100° C. and larger mass losses above 150° C., potentially attributed to decomposition. ¹H NMR showed the Formula I:S-Mandelic acid ratio was 1.0:0.6, and the IPAc content was 2.2 wt %.

### Formula I Gentisate

A 4 mL threaded glass vial was charged with about 0.2 g of Formula I, about 2 eq. of gentisic acid, and about 3 mL of isopropyl acetate. The vial was capped and agitated at room temperature for about 30 days. Solids were isolated by centrifugation.

Formula I Gentisate was characterized by XRPD, DSC, TGA, and ¹H NMR. The resulting XRPD diffractogram of freshly prepared solids (Formula I Gentisate wet cake) is shown in FIG. 68. An XRPD peak list is reported in Table 35.

TABLE 35

| | Position | Relative Intensity |
|---|---|---|
| No. | °2-theta | (%) |
| 1 | 7.4 | 100.0 |
| 2 | 6.5 | 96.4 |
| 3 | 8.0 | 45.7 |
| 4 | 13.5 | 16.4 |
| 5 | 16.9 | 36.0 |
| 6 | 12.0 | 26.9 |
| 7 | 25.1 | 13.1 |
| 8 | 22.4 | 14.5 |
| 9 | 19.3 | 19.8 |

Formula I Gentisate wet cake, described above, was air-dried. The resulting solid form was characterized by XRPD. The resulting XRPD diffractogram is shown in FIG. 69. An XRPD peak list is reported in Table 36.

TABLE 36

| | Position | Relative Intensity |
|---|---|---|
| No. | °2-theta | (%) |
| 1 | 7.4 | 100.0 |
| 2 | 6.1 | 71.5 |
| 3 | 8.4 | 71.4 |
| 4 | 16.7 | 59.8 |
| 5 | 17.9 | 41.5 |
| 6 | 9.1 | 38.4 |
| 7 | 24.9 | 30.5 |
| 8 | 22.8 | 19.4 |
| 9 | 12.3 | 16.3 |

DSC thermogram of air-dried Formula I Gentisate was conducted and the resulting thermogram is shown in FIG. 70. TGA of air-dried Formula I Gentisate was performed and the resulting thermogram is depicted in FIG. 71. The TGA showed a mass loss of about 1.5 weight percent up to about 100° C. and larger mass losses above 150° C., potentially due to decomposition ¹H NMR showed the Formula I: Gentisic acid ratio was 1.0:0.8, and the IPAc content was 2.6 wt %.

### Formula I Citrate

A 4 mL threaded glass vial was charged with about 0.2 g of Formula I, about 2 eq. of citric acid, and about 3 mL of isopropyl acetate. The vial was capped and agitated at room temperature for about 30 days. Solids were isolated by centrifugation for testing.

Formula I Citrate was characterized by XRPD, DSC, TGA, and ¹H NMR. The resulting XRPD diffractogram of freshly prepared solids (wet cake) is shown in FIG. 72. An XRPD peak list is reported in Table 37.

TABLE 37

| | Position | Relative Intensity |
|---|---|---|
| No. | °2-theta | (%) |
| 1 | 7.4 | 100.0 |
| 2 | 7.9 | 26.4 |
| 3 | 16.7 | 33.0 |
| 4 | 15.1 | 7.4 |
| 5 | 17.7 | 13.8 |
| 6 | 20.4 | 17.6 |
| 7 | 11.4 | 4.1 |
| 8 | 18.9 | 14.5 |
| 9 | 22.1 | 15.2 |

Formula I Citrate described above was air-dried. The resulting solid form was characterized by XRPD (FIG. 73). An XRPD peak list is reported in Table 38.

TABLE 38

| | Position | Relative Intensity |
|---|---|---|
| No. | °2-theta | (%) |
| 1 | 6.1 | 58.5 |
| 2 | 7.4 | 92.1 |
| 3 | 18.2 | 100.0 |
| 4 | 8.3 | 44.2 |
| 5 | 16.4 | 60.1 |
| 6 | 36.7 | 48.4 |
| 7 | 20.7 | 36.4 |
| 8 | 24.5 | 18.8 |
| 9 | 26.0 | 22.7 |

DSC was conducted on air-dried Formula I Citrate and the resulting thermogram is depicted in FIG. 74. TGA of Formula I Citrate Form B was performed and the resulting thermogram is depicted in FIG. 75, which showed a mass loss of about 2.3 weight percent up to about 100° C. and larger mass losses above 150° C., likely due to decomposition. ¹H NMR showed the Formula I: Citric acid ratio is 1.0:0.8, and the IPAc content is 0.3 wt %.

### Formula I R-Mandelate

A 4 mL threaded glass vial was charged with about 0.2 g of Formula I, about 2 eq. of R-Mandelic acid, and about 3 mL of isopropyl acetate. The vial was capped and agitated at room temperature for about 30 days. Solids were isolated by centrifugation for testing.

Formula I R-Mandelate was characterized by XRPD, DSC, TGA, and ¹H NMR. The resulting XRPD diffractogram of freshly prepared solids (Formula I R-Mandelate Form A) is shown in FIG. 76. An XRPD peak list is reported in Table 39.

TABLE 39

| | Position | Relative Intensity |
|---|---|---|
| No. | °2-theta | (%) |
| 1 | 5.7 | 100.0 |
| 2 | 5.4 | 83.3 |
| 3 | 16.5 | 37.0 |
| 4 | 6.2 | 22.0 |
| 5 | 7.8 | 9.2 |
| 6 | 11.5 | 19.1 |
| 7 | 10.5 | 12.8 |
| 8 | 13.4 | 17.0 |
| 9 | 18.5 | 14.1 |

Formula I R-Mandelate Form A, described above, was air-dried. The resulting solid form, Formula I R-Mandelate Form B, was characterized by XRPD. The XRPD diffractogram of Formula I R-Mandelate Form B is shown in FIG. 77. An XRPD peak list is reported in Table 40.

TABLE 40

| | Position | Relative Intensity |
|---|---|---|
| No. | °2-theta | (%) |
| 1 | 6.2 | 100.0 |
| 2 | 8.3 | 8.9 |
| 3 | 22.4 | 19.7 |
| 4 | 15.9 | 10.3 |
| 5 | 16.3 | 8.7 |
| 6 | 17.0 | 11.4 |
| 7 | 10.0 | 5.8 |
| 8 | 10.8 | 8.3 |
| 9 | 11.6 | 12.3 |

DSC of Formula I R-Mandelate Form B was conducted and the resulting thermogram is depicted in FIG. 78 and shows a broad endothermic event from about 50 to 75° C., a sharp endothermic event at about 150° C., and endothermic and exothermic events at higher temperature, potentially due to decomposition. TGA of Formula I R-Mandelate Form B was performed and the resulting thermogram is shown in FIG. 79. The TGA showed a mass loss of about 3.3 weight percent up to about 100° C. and larger mass losses above 150° C., potentially due to decomposition. ¹H NMR showed Formula I: RMandelic acid ratio was about 1:0.5 and about 0.7 wt % IPAc.

### Formula I Benzoate

A 4 mL threaded glass vial was charged with about 0.2 g of Formula I, about 2 eq. of Benzoic acid, and about 3 mL of isopropyl acetate. The vial was capped and agitated at room temperature for about 30 days. Solids were isolated by centrifugation for testing.

Formula I Benzoate was characterized by XRPD, DSC, TGA, and ¹H NMR. The resulting XRPD diffractogram of freshly prepared solids (wet cake) (Formula I Benzoate Form A) is shown in FIG. 80. An XRPD peak list is reported in Table 41.

TABLE 41

| | Position | Relative Intensity |
|---|---|---|
| No. | °2-theta | (%) |
| 1 | 7.44 | 100.00 |
| 2 | 18.97 | 33.75 |
| 3 | 16.70 | 41.50 |
| 4 | 21.14 | 26.17 |
| 5 | 22.45 | 23.59 |
| 6 | 25.07 | 23.94 |
| 7 | 6.09 | 20.93 |

Formula I Benzoate Form A, described above, was air-dried. The resulting solid form, Formula I Benzoate Form B, was characterized. The XRPD diffractogram of Formula I Benzoate Form B is shown in FIG. 81. An XRPD peak list is reported in Table 42.

TABLE 42

| | Position | Relative Intensity |
|---|---|---|
| No. | °2-theta | (%) |
| 1 | 14.9 | 34.0 |
| 2 | 6.7 | 29.0 |
| 3 | 7.4 | 100.0 |
| 4 | 20.6 | 40.3 |
| 5 | 22.5 | 43.1 |
| 6 | 8.1 | 43.3 |
| 7 | 6.7 | 29.0 |
| 8 | 14.9 | 34.0 |
| 9 | 21.5 | 34.8 |

DSC thermogram of Formula I Benzoate Form B was conducted and the resulting thermogram is depicted in FIG. 82. TGA of Formula I Benzoate Form B was performed and the resulting thermogram is shown in FIG. 83. ¹H NMR showed the Formula I: Benzoic acid ratio is about 1.0:0.5, and the IPAc content is about 1.9 wt %.

### Formula I Methylparabenate

A 4 mL threaded glass vial was charged with about 0.2 g of Formula I, about 2 eq. of Methylparaben, and about 3 mL of isopropyl acetate. The vial was capped and agitated at room temperature for about 30 days. Solids were isolated by centrifugation.

Formula I Methylparabenate was characterized by XRPD, DSC, TGA, and ¹H NMR. The resulting XRPD diffractogram of freshly prepared solids (wet cake) (Formula I Methylparabenate Form A) is shown in FIG. 85. An XRPD peak list is reported in Table 43.

TABLE 43

| | Position | Relative Intensity |
|---|---|---|
| No. | °2-theta | (%) |
| 1 | 7.4 | 100 |
| 2 | 8.0 | 82.5 |
| 3 | 6.3 | 45.7 |
| 4 | 20.5 | 36.8 |
| 5 | 19.2 | 19.2 |
| 6 | 12.1 | 20.0 |
| 7 | 31.6 | 10.6 |
| 8 | 22.8 | 27.6 |
| 9 | 14.0 | 19.4 |

Formula I Methylparabenate Form A, described above, was air-dried. The resulting solid form, Formula I Methylparabenate Form B, was characterized by XRPD (FIG. 85). An XRPD peak list is reported in Table 44.

TABLE 44

| | Position | Relative Intensity |
|---|---|---|
| No. | °2-theta | (%) |
| 1 | 7.4 | 100.0 |
| 2 | 8.3 | 71.0 |
| 3 | 6.5 | 33.1 |
| 4 | 19.3 | 51.1 |
| 5 | 20.6 | 64.4 |
| 6 | 21.6 | 69.7 |
| 7 | 12.2 | 27.4 |
| 8 | 13.0 | 18.3 |
| 9 | 31.8 | 26.6 |

The DSC thermogram of Formula I Methylparabenate Form B was conducted and the thermogram is depicted in FIG. 86. TGA of Formula I Methylparabenate Form B was performed and the resulting thermogram is shown in FIG. 87. ¹H NMR showed the Formula I: Methylparaben ratio was about 1.0:0.7, and the IPAc content was about 0 wt %.

### Formula I Caffeate

A 4 mL threaded glass vial was charged with about 0.2 g of Formula I, about 2 eq. of caffeic acid, and about 3 mL of isopropyl acetate. The vial was capped and agitated at room temperature for about 30 days. Solids were isolated by centrifugation.

Formula I Caffeate was characterized by XRPD, DSC, TGA, and ¹H NMR. The resulting XRPD diffractogram of freshly prepared solids (Formula I Caffeate wet cake) is shown in FIG. 88. An XRPD peak list is reported in Table 45.

TABLE 45

| | Position | Relative Intensity |
|---|---|---|
| No. | °2-theta | (%) |
| 1 | 5.3 | 100 |
| 2 | 7.4 | 20.6 |
| 3 | 9.1 | 21.7 |
| 4 | 6.5 | 12.4 |
| 5 | 7.0 | 10.2 |
| 6 | 15.9 | 19.0 |
| 7 | 27.1 | 19.0 |
| 8 | 22.5 | 16.1 |
| 9 | 10.6 | 7.7 |

Formula I Caffeate wet cake, described above, was air-dried and characterized by XRPD. The resulting XRPD diffractogram is shown in FIG. 89. DSC of air-dried Formula I Caffeate was conducted and the resulting thermogram is shown in FIG. 90. TGA was performed and the resulting thermogram is depicted in FIG. 91. ¹H NMR showed Formula I: Caffeic Acid ratio was about 1.0:1.3.

### Formula I Glycolate

A 4 mL threaded glass vial was charged with a stir bar, about 0.9 g of Formula I Form II, 1.0 eq. of Glycolic acid, and 2 mL of ethyl acetate. The suspension was stirred at room temperature for about 6 days and sampled for further analysis. Solids for XRPD characterization were isolated by centrifugation.

Formula I Glycolate was characterized by XRPD, ¹H NMR, and DVS. The resulting XRPD diffractogram of the freshly prepared solids (wet cake) is shown in FIG. 92. An XRPD peak list is reported in Table 46.

TABLE 46

| | Position | Relative Intensity |
|---|---|---|
| No. | °2-theta | (%) |
| 1 | 6.2 | 100.0 |
| 2 | 8.4 | 13.7 |
| 3 | 11.5 | 16.6 |
| 4 | 10.9 | 10.8 |
| 5 | 16.4 | 33.2 |
| 6 | 22.3 | 14.5 |
| 7 | 13.6 | 11.3 |
| 8 | 14.0 | 6.7 |
| 9 | 15.3 | 9.2 |

Formula I Glycolate wet cake, described above, was vacuum-dried. The resulting solid form was characterized by XRPD and the resulting diffractogram is shown in FIG. 93. An XRPD peak list is reported in Table 47.

TABLE 47

| | Position | Relative Intensity |
|---|---|---|
| No. | °2-theta | (%) |
| 1 | 6.2 | 100.0 |
| 2 | 8.3 | 12.5 |
| 3 | 11.5 | 12.2 |
| 4 | 10.8 | 9.5 |
| 5 | 16.4 | 33.2 |
| 6 | 22.2 | 13.2 |
| 7 | 13.6 | 6.5 |
| 8 | 14.9 | 6.3 |
| 9 | 15.3 | 7.7 |

DVS of dried Formula I Glycolate was performed and showed a weight change of about 7.5% from 0-90% RH. Weak hysteresis was observed, and the behavior was roughly linear. Weight change from 50-90% RH was about 2%. ¹H NMR showed the Formula I: Glycolic acid ratio was 1.0:0.8.

### Formula I α-Ketobutyrate

A flask was charged with a stir bar, about 5.0 g of Formula I Form I, 1.0 eq. of α-ketobutyric acid, and 10 mL of ethyl acetate. The suspension was stirred at room temperature for about 3 days and sampled for testing. The suspension was filtered and washed with about 2 mL of ethyl acetate the next day, air dried for about 1 day, then dried under vacuum at about 40° C. for about 7 days.

Formula I α-Ketobutyrate was characterized by XRPD, ¹H NMR, and DVS. The resulting XRPD diffractogram of the wet cake Formula I α-Ketobutyrate is shown in FIG. 94. An XRPD peak list is reported in Table 48.

TABLE 48

| | Position | Relative Intensity |
|---|---|---|
| No. | °2-theta | (%) |
| 1 | 6.2 | 100.0 |
| 2 | 8.3 | 11.3 |
| 3 | 10.8 | 10.1 |
| 4 | 12.3 | 1.6 |
| 5 | 12.5 | 1.5 |
| 6 | 12.7 | 1.9 |
| 7 | 16.4 | 6.9 |
| 8 | 19.0 | 6.3 |
| 9 | 21.6 | 5.0 |

Formula I α-Ketobutyrate described above, was air- and characterized by XRPD (FIG. 95). An XRPD peak list of the dry material is reported in Table 49.

TABLE 49

| | Position | Relative Intensity |
|---|---|---|
| No. | °2-theta | (%) |
| 1 | 6.2 | 100.0 |
| 2 | 16.4 | 15.8 |
| 3 | 8.3 | 15.1 |
| 4 | 10.8 | 11.8 |
| 5 | 11.5 | 12.6 |
| 6 | 20.3 | 12.4 |
| 7 | 18.5 | 5.9 |
| 8 | 19.0 | 5.7 |
| 9 | 21.6 | 7.1 |

DVS isotherm of Formula I α-Ketobutyrate Form B was performed, and the resulting isotherm showed a weight change of about 5.5% from 0-90% RH. Hysteresis was minor from 10-90% RH, but slope change was observed between 0 and 10% RH. Weight change from 50-90% RH was about 2%. ¹H NMR of the washed wet cake solids showed the Formula I: Glycolic acid ratio was 1.0:0.8.

### Formula I Pyruvate

A threaded glass vial was charged with about 5 grams of Formula I Form I, about 1.0 eq. of Pyruvic acid, and about 10 mL of ethyl acetate. The vial was capped, and the suspension was magnetically stirred at room temperature for about 5 days. The suspension was filtered, and the cake was washed with about 2 mL of ethyl acetate. The cake was dried under vacuum at room temperature and the product was isolated.

Formula I Pyruvate was characterized by XRPD, ¹H NMR, and DVS. The resulting XRPD diffractogram of freshly prepared solids (wet cake) is shown in FIG. 96. An XRPD peak list is reported in Table 50.

TABLE 50

| | Position | Relative Intensity |
|---|---|---|
| No. | °2-theta | (%) |
| 1 | 6.21 | 100.00 |
| 2 | 8.34 | 13.30 |
| 3 | 10.85 | 11.29 |
| 4 | 11.56 | 8.27 |
| 5 | 13.60 | 6.37 |
| 6 | 14.51 | 5.59 |
| 7 | 20.32 | 10.59 |
| 8 | 21.73 | 7.08 |
| 9 | 22.59 | 5.13 |

Formula I Pyruvate, described above, was vacuum dried and subjected to XRPD. The XRPD diffractogram is shown in FIG. 97. An XRPD peak list is reported in Table 51.

TABLE 51

| | Position | Relative Intensity |
|---|---|---|
| No. | °2-theta | (%) |
| 1 | 6.2 | 100.0 |
| 2 | 8.3 | 15.0 |
| 3 | 10.8 | 10.8 |
| 4 | 11.5 | 16.1 |
| 5 | 16.3 | 20.1 |
| 6 | 19.1 | 9.1 |
| 7 | 20.3 | 17.9 |
| 8 | 21.7 | 11.4 |
| 9 | 22.1 | 10.8 |

DVS of Formula I Pyruvate Form B was performed, and the resulting isotherm showed weight change of about 4.3% from 10-90% RH with weak hysteresis and linear behavior. Weight change from 50 to 90% RH is about 2.5%. ¹H NMR of solids from the washed filter cake showed a Formula I: Pyruvic acid ratio of 1.0:0.90.

### Stability Study

Formula I amorphous material and Forms I, II, III and IV were examined for loss of purity over time as determined by liquid chromatography. As observed by the data present in Table 57, Formula I Forms II, III, and IV have a decreased loss of purity over time when compared to Formula I Form I or Formula I amorphous material.

The sample preparations for the stability study are described as follows:
Formula I Amorphous: A sample of amorphous solids was placed at about 22° C. open to air and the purity was monitored over 3 days by liquid chromatography.
Formula I Form I: Samples of Formula I Form I were placed in sealed glass vials and placed in a chamber at about 40° C. and about 75% RH and the purity was periodically monitored by liquid chromatography.
Formula I Form II: Samples of Formula I Form II were placed in an open vial. The vial was placed inside a container alongside a saturated salt solution to provide a humidity of about 88% RH and sealed. The sealed container was then placed into a chamber at about 40° C. and the purity was periodically monitored by liquid chromatography.
Formula I Form III: Samples of Formula I Form II were placed in an open vial. The vial was placed inside a container alongside a saturated salt solution to provide a humidity of about 27% RH and sealed. The sealed container was then placed into a chamber at about 40° C. and the purity was periodically monitored by liquid chromatography.
Formula I Form IV: Samples of Formula I Form IV were packaged at about 0% RH inside a PE bag and heat-sealed inside a foil pouch. The pouches were then placed in a chamber at about 40° C. and the purity was periodically monitored by liquid chromatography.

The results are reported in Table 52.

TABLE 52

| | Formula I Purity Over Time (% purity) | | | | | |
|---|---|---|---|---|---|---|
| | | Formula I | Formula I | Formula I | Formula I | Formula I |
| | | Amorphous | Form I | Form II | Form III | Form IV |
| Duration | Solid Form | About 22° | About 40° | About 40° | About 40° | About 40° |
| Time | Storage | C., open to | C., about | C., about | C., about | C., about |
| (days) | Conditions | atmosphere | 75% RH | 88% RH | 27% RH | 0% RH |
| 0 | | 95.74 | 99.13 | 98.78 | 98.61 | 99.49 |
| 1 | | 95.45 | — | 98.67 | 98.58 | — |
| 2 | | 95.18 | — | — | — | — |
| 3 | | 95.05 | — | — | — | — |
| 7 | | — | 98.37 | 98.64 | 98.55 | — |
| 14 | | — | 97.97 | 98.59 | 98.50 | 99.44 |
| 28 | | — | 96.57 | — | — | 99.43 |

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A solid form of Formula I Form II, wherein the Formula I has the structure:

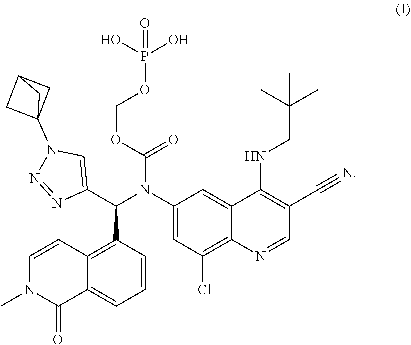 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

2. The solid form of claim 0, characterized by an X-ray diffraction pattern having 2θ-reflections at 7.4, 9.4, and 10.6 degrees 2θ, plus or minus 0.2 degrees 2θ.

3. The solid form of claim 0, characterized by an X-ray diffraction pattern further comprising 2θ-reflections at 8.8, 12.3, and 26.1 degrees 2θ, plus or minus 0.2 degrees 2θ.

4. The solid form of any claim 3, characterized by an X-ray diffraction pattern further comprising 2θ-reflections at 14.7, 18.1, and 22.4 degrees 2θ, plus or minus 0.2 degrees 2θ.

5. The solid form of claim 1, having an X-ray diffraction pattern substantially as shown in FIG. 7.

6. The solid form of claim 1, having a whole pattern Pawley Refinement diffractogram substantially as shown in FIG. 8.

7. A solid form of Formula I Form III, wherein the Formula I has the structure:

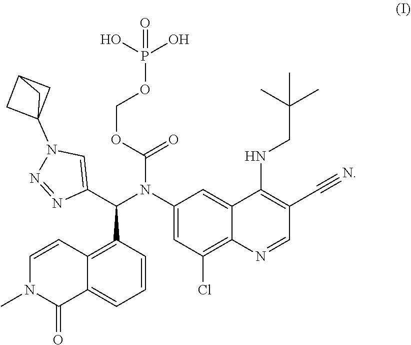 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

8. The solid form of claim 0, characterized by an X-ray diffraction pattern having 2θ-reflections at 7.8, 9.8, and 10.7 degrees 2θ, plus or minus 0.2 degrees 2θ.

9. The solid form of claim 0, characterized by an X-ray diffraction pattern further comprising 2θ-reflections at 8.9, 12.5, and 20.1 degrees 2θ, plus or minus 0.2 degrees 2θ.

10. The solid form of claim 9, characterized by an X-ray diffraction pattern further comprising 2θ-reflections at 15.5, 18.2, and 22.9 degrees 2θ, plus or minus 0.2 degrees 2θ.

11. The solid form of claim 7, having an X-ray diffraction pattern substantially as shown in FIG. 11.

12. The solid form of claim 7, having a dynamic vapor sorption isotherm substantially as shown in FIG. 14.

13. A solid form of Formula I Form IV, wherein the Formula I has the structure:

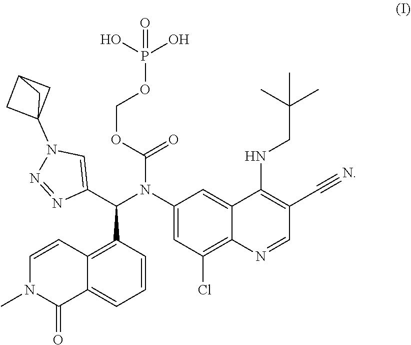 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

14. The solid form of claim 0, characterized by an X-ray diffraction pattern having 2θ-reflections at 8.0, 18.1, and 20.0 degrees 2θ, plus or minus 0.2 degrees 2θ.

15. The solid form of claim 0, characterized by an X-ray diffraction pattern further comprising 2θ-reflections at 9.0, 9.9, and 10.8 degrees 2θ, plus or minus 0.2 degrees 2θ.

16. The solid form of claim 15, characterized by an X-ray diffraction pattern further comprising 2θ-reflections at 15.6, 22.8, and 24.9 degrees 2θ, plus or minus 0.2 degrees 2θ.

17. The solid form of claim 13, having an X-ray diffraction pattern substantially as shown in FIG. 16.

18. A solid form of Formula I Form VII, wherein the Formula I has the structure:

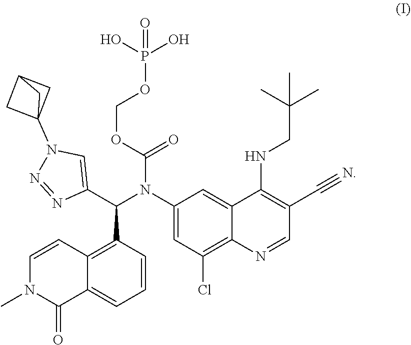 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

19. The solid form of claim 0, characterized by an X-ray diffraction pattern having 2θ-reflections at 6.2, 8.4, and 22.6 degrees 2θ, plus or minus 0.2 degrees 2θ.

20. The solid form of claim 0, characterized by an X-ray diffraction pattern further comprising 2θ-reflections at 10.3, 10.9, and 11.7 degrees 2θ, plus or minus 0.2 degrees 2θ.

21. The solid form of claim 20, characterized by an X-ray diffraction pattern further comprising 2θ-reflections at 16.1, 16.4, and 17.3 degrees 2θ, plus or minus 0.2 degrees 2θ.

22. The solid form of claim 18, having an X-ray diffraction pattern substantially as shown in FIG. 22.

23. The solid form of claim 18, having a mass loss of about 2.9 wt % at a temperature from about 50-125° C. when subjected to thermogravimetric analysis—mass spectrometry.

24. A solid form of Formula I Form IX, wherein the Formula I has the structure:

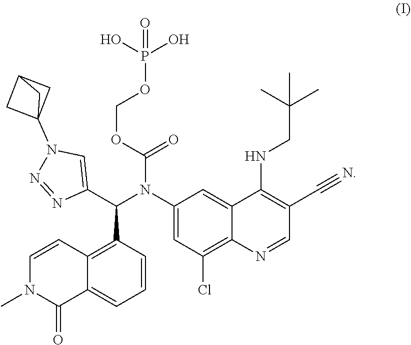 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

25. The solid form of claim 0, characterized by an X-ray diffraction pattern having 2θ-reflections at 7.2, 5.8, and 5.7 degrees 2θ, plus or minus 0.2 degrees 2θ.

26. The solid form of claim 0, characterized by an X-ray diffraction pattern further comprising 2θ-reflections at 10.7, 15.3, and 17.1 degrees 2θ, plus or minus 0.2 degrees 2θ.

27. The solid form of claim 26, characterized by an X-ray diffraction pattern further comprising 2θ-reflections at 7.4, 10.0, and 8.9 degrees 2θ, plus or minus 0.2 degrees 2θ.

28. The solid form of claim 24, having an X-ray diffraction pattern substantially as shown in FIG. 24.

29. The Formula I Form IX of claim 24, having a mass loss of about 6.3% at 30° C. when subjected to thermogravimetric analysis—Mass Spectrometry.

30. A solid form of Formula I S-Mandelate Form A, wherein the Formula I has the structure:

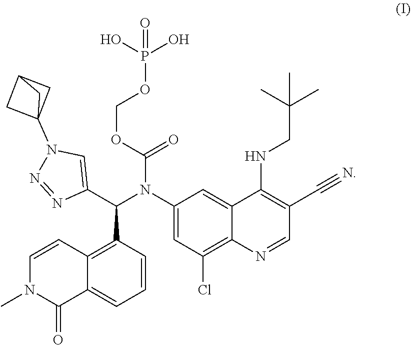 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

31. The solid form of claim 0, characterized by an X-ray diffraction pattern having 2θ-reflections at 5.5, 5.7, and 6.3 degrees 2θ, plus or minus 0.2 degrees 2θ.

32. The solid form of claim 0, characterized by an X-ray diffraction pattern further comprising 2θ-reflections at 11.4, 16.6, and 20.8 degrees 2θ, plus or minus 0.2 degrees 2θ.

33. The solid form of claim 32, characterized by an X-ray diffraction pattern further comprising 2θ-reflections at 10.0, 10.4, and 19.0 degrees 2θ, plus or minus 0.2 degrees 2θ.

34. The solid form of claim 30, having an X-ray diffraction pattern substantially as shown in FIG. 64.

35. A solid form of Formula I S-Mandelate Form B, wherein the Formula I has the structure:

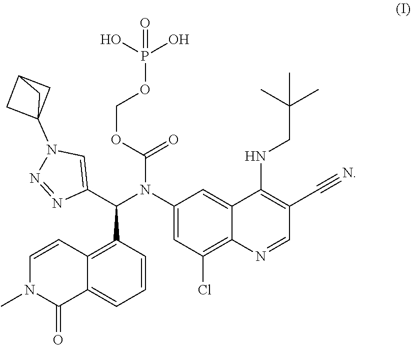 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

36. The solid form of claim 0, characterized by an X-ray diffraction pattern having 2θ-reflections at 5.7, 6.2, and 22.6 degrees 2θ, plus or minus 0.2 degrees 2θ.

37. The solid form of claim 0, characterized by an X-ray diffraction pattern further comprising 2θ-reflections at 8.0, 8.4, and 11.7 degrees 2θ, plus or minus 0.2 degrees 2θ.

38. The solid form of claim 37, characterized by an X-ray diffraction pattern further comprising 2θ-reflections at 13.5, 16.0, and 16.6 degrees 2θ, plus or minus 0.2 degrees 2θ.

39. The solid form of claim 35, having an X-ray diffraction pattern substantially as shown in FIG. 65.

40. The solid form of claim 35, having a differential scanning calorimetry thermogram comprising a first endothermic event from about 50° C. to about 75° C., and a second endothermic event at about 150° C.

41. The solid form of claim 35, having a differential scanning calorimetry thermogram substantially as shown in FIG. 66.

42. The solid form of claim 35, having a thermogravimetric analysis substantially as shown in FIG. 67.

43. A solid form of Formula I R-Mandelate Form A, wherein the Formula I has the structure:

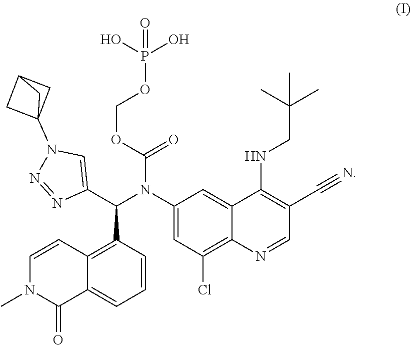 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

44. The solid form of claim 0, characterized by an X-ray diffraction pattern having 2θ-reflections at 5.7, 5.4, and 16.5 degrees 2θ, plus or minus 0.2 degrees 2θ.

45. The solid form of claim 0, characterized by an X-ray diffraction pattern further comprising 2θ-reflections at 6.2, 7.8, and 11.5 degrees 2θ, plus or minus 0.2 degrees 2θ.

46. The solid form of claim 45, characterized by an X-ray diffraction pattern further comprising 2θ-reflections at 10.5, 13.4, and 18.5 degrees 2θ, plus or minus 0.2 degrees 2θ.

47. The solid form of claim 43, having an X-ray diffraction pattern substantially as shown in FIG. 92.

48. A solid form of Formula I R-Mandelate Form B, wherein the Formula I has the structure:

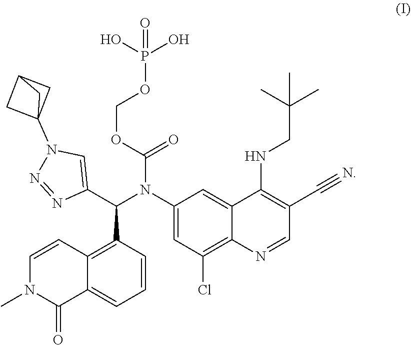 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]

49. The solid form of claim 0, characterized by an X-ray diffraction pattern having 2θ-reflections at 6.2, 8.3, and 22.4 degrees 2θ, plus or minus 0.2 degrees 2θ.

50. The solid form of claim 0, characterized by an X-ray diffraction pattern further comprising 2θ-reflections at 15.9, 16.3, and 17.0 degrees 2θ, plus or minus 0.2 degrees 2θ.

51. The solid form of claim 50, characterized by an X-ray diffraction pattern further comprising 2θ-reflections at 10.0, 10.8, and 11.6 degrees 2θ, plus or minus 0.2 degrees 2θ.

52. The solid form of claim 48, having an X-ray diffraction pattern substantially as shown in FIG. 77.

53. The solid form of claim 48, having a differential scanning calorimetry thermogram comprising a first endotherm with onset at about 50° C., and a second endotherm with onset at about 150° C.

54. The solid form of claim 48, having a differential scanning calorimetry thermogram substantially as shown in FIG. 78.

55. The solid form of claim 48, having a thermogravimetric analysis substantially as shown in FIG. 79.

56. A pharmaceutical composition comprising a therapeutically effective amount of the solid form of claim 1 and a pharmaceutically acceptable carrier.

57. The pharmaceutical composition of claim 0, further comprising one to three additional therapeutic agents.

58. The pharmaceutical composition of claim 0, wherein at least one of the additional therapeutic agents is active against an inflammatory disease.

59. The pharmaceutical composition of claim 56, wherein the pharmaceutical composition is in a unit dosage form.

60. The pharmaceutical composition of claim 0, wherein the unit dosage form is a tablet.

61. (canceled)

62. (canceled)

63. A pharmaceutical composition comprising a therapeutically effective amount of the solid form of claim 7, and a pharmaceutically acceptable carrier.

64. The pharmaceutical composition of claim 63, further comprising one to three additional therapeutic agents.

65. The pharmaceutical composition of claim 64, wherein at least one of the additional therapeutic agents is active against an inflammatory disease.

66. The pharmaceutical composition of claim 63, wherein the pharmaceutical composition is in a unit dosage form.

67. The pharmaceutical composition of claim 66, wherein the unit dosage form is a tablet.

68. A pharmaceutical composition comprising a therapeutically effective amount of the solid form of claim 13, and a pharmaceutically acceptable carrier.

69. The pharmaceutical composition of claim 68, further comprising one to three additional therapeutic agents.

70. The pharmaceutical composition of claim 69, wherein at least one of the additional therapeutic agents is active against an inflammatory disease.

71. The pharmaceutical composition of claim 68, wherein the pharmaceutical composition is in a unit dosage form.

72. The pharmaceutical composition of claim 71, wherein the unit dosage form is a tablet.

73. A pharmaceutical composition comprising
a therapeutically effective amount of a mixture of a solid form of Formula I Form II, a solid form of Formula I Form III, and a solid form of Formula I Form IV; and
a pharmaceutically acceptable carrier, wherein the Formula I has the structure: 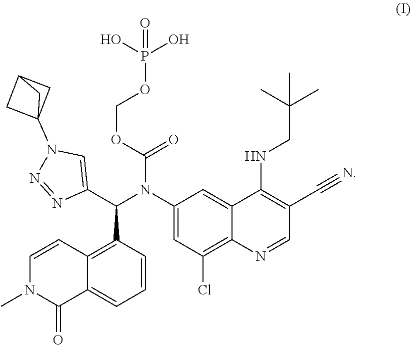 [SMILES: Cn1ccc2c([C@@H](c3cn(C45CC(C4)C5)nn3)N(C(=O)OCOP(=O)(O)O)c3cc(Cl)c4ncc(C#N)c(NCC(C)(C)C)c4c3)cccc2c1=O]
